(19) United States
(12) Patent Application Publication
(10) Pub. No.: US 2010/0035756 A1
(43) Pub. Date: Feb. 11, 2010

(54) TRIAZOLOPHYRIDINE DERIVATIVES AS HERBICIDES

(75) Inventors: Christoph Luthy, Basel (CH); Roger Graham Hall, Basel (CH); Andrew Edmunds, Basel (CH); Suzanna Riley, Berkshire (GB); Martin Diggelmann, Berkshire (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignees: SYNGENTA LIMITED, Guildford, Surrey (GB); SYNGENTA CROP PROTECTION, INC., Greensboro, NC (US)

(21) Appl. No.: 12/373,309

(22) PCT Filed: Jul. 10, 2007
(86) PCT No.: PCT/EP07/06086

§ 371 Date: Aug. 7, 2009

(30) Foreign Application Priority Data
Jul. 12, 2006 (EP) 06014495.3

(51) Int. Cl. A01N 43/40; C07D 471/04; A01P 13/00

(52) U.S. Cl. 504/246; 546/120

(57) ABSTRACT

Compounds of formula (I), wherein the substituents are as defined in claim 1, are suitable for use as herbicides.

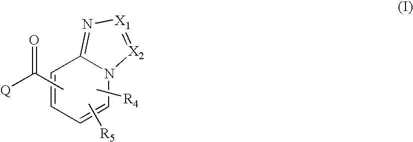

The present invention relates to novel, herbicidally active bicylic heteroaryl derivatives, to processes for their preparation, to compositions comprising those compounds, and to their use in controlling weeds, especially in crops of useful plants, or for inhibiting plant growth.

Nitrogen containing heterocyclyl derivatives having herbicidal action are described, for example, in EP 0283261 A1. Novel nicotinoyl derivatives having herbicidal and growth-inhibiting properties have now been found.

The present invention accordingly relates to compounds of formula I

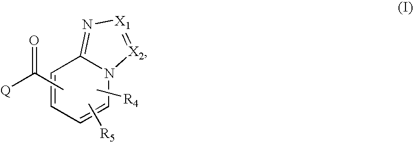

wherein

X₁ is nitrogen, if X₂ is CR₂; or CR₁, if X₂ is nitrogen; or NR₅₁, if X₂ is C(O); or C(O), if X₂ is NR₅₂;

X₂ is nitrogen, if X₁ is CR₁; or CR₂, if X₁ is nitrogen; or NR₅₂, if X₁ is C(O); or C(O), if X₁ is NR₅₁;

R₅₁ and R₅₂ independently from each other, are hydrogen, a group —X₆ or a group —X₄—X₅—X₆;

R₁ and R₂ independently from each other, are hydrogen, halogen, hydroxy, mercapto, amino, azido, SF₅, nitro, cyano, rhodano, carbamoyl, carboxy, formyl, tri(C₁-C₄alkyl)silyl, C₁-C₄alkyl(C₁-C₄alkoxy)phosphino or di(C₁-C₄alkoxy)phosphono;

or R₁ and R₂ independently from each other are a group —X₆, a group —X₅—X₆ or a group —X₄—X₅—X₆, wherein

X₄ is C₁-C₆alkylene, C₂-C₆alkenylene or C₂-C₆alkynylene, which can be mono- or poly-substituted by halogen, hydroxy, C₁-C₆alkoxy, C₃-C₆cycloalkyloxy, C₁-C₆alkoxy-C₁-C₆alkoxy, C₁-C₆alkoxy-C₁-C₆alkoxy-C₁-C₆alkoxy or C₁-C₂alkylsulfonyloxy; or by a bivalent C₁-C₈ alkylene group which may be interrupted by 1 to 2 oxygen atoms, sulphur or NRa₂₆, said bivalent C₁-C₈alkylene group can be substituted by substituents from the group consisting of halogen, hydroxy, mercapto, amino, formyl, carboxy, nitro, cyano, carbamoyl, C₁-C₆alkoxy, C₁-C₆alkoxycarbonyl, C₁-C₆-alkylaminocarbonyl, C₁-C₆-dialkylaminocarbonyl, C₂-C₆alkenyl, C₂-C₆haloalkenyl, C₂-C₆alkynyl, C₂-C₆haloalkynyl, C₂-C₆alkenyloxy, C₂-C₆alkynyloxy, C₁-C₆haloalkoxy, C₂-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆alkoxy, formyloxy, C₁-C₆alkylcarbonyloxy, C₁-C₆alkylcarbonyl, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, C₁-C₆alkylthiocarbonyl, C₁-C₆alkylamino, di(C₁-C₆alkyl)amino, C₁-C₄alkylsulfonyloxy, C₁-C₄alkylcarbonylamino, N(C₁-C₄alkyl)-C₁-C₄alkylcarbonylamino, C₁-C₄alkoxycarbonylamino, N(C₁-C₄alkyl)-C₁-C₄alkoxycarbonylamino, C₁-C₄alkylsulfonylamino, N(C₁-C₄alkyl)-C₁-C₄alkylsulfonylamino, OSO₂—C₁-C₄-alkyl, rhodano, tri(C₁-C₄alkyl)silyl, C₁-C₄alkyl(C₁-C₄alkoxy)phosphino and di(C₁-C₄alkoxy)phosphono;

X₅ is oxygen, —OC(O)—, —OC(O)O—, —OC(O)N(R₃)—, —ON(Ra₂₁)—, —ON═C(Ra₂₂)—, OS(O)₂—, OS(O)₂O—, OS(O)₂N(R₃)—, thio, sulfinyl, sulfonyl, —SO₂N(R₃)—, —S(O)₂O—, —S(═NRa₂₃)(O)—, —C(O)O—, —C(O)—, —C(O)N(R₃)—, —C(Ra₂₂)═NO—, —N(Ra₂₁)O—, —N(R₃)SO₂—, —N(Ra₂₄)—, —N(R₃)C(O)—, —N(R₃)C(O)O—, —N(R₃)C(O)N(R₃)—, —N(R₃)SO₂O—, —N(R₃)SO₂N(R₃)—, —N═S(Ra₂₅)(O)— or —S(Ra₂₅)(O)═N—;

X₆ is C₁-C₆alkyl, C₂-C₆alkenyl or C₂-C₆alkynyl; or C₁-C₆alkyl, C₂-C₆alkenyl or C₂-C₆alkynyl mono- or poly-substituted by halogen, hydroxy, mercapto, amino, formyl, carboxy, nitro, cyano, carbamoyl, C₁-C₆alkoxy, C₁-C₆alkoxycarbonyl, C₁-C₆-alkylaminocarbonyl, C₁-C₆-dialkylaminocarbonyl, C₂-C₆alkenyl, C₂-C₆haloalkenyl, C₂-C₆alkynyl, C₂-C₆haloalkynyl, C₂-C₆alkenyloxy, C₂-C₆alkynyloxy, C₁-C₆haloalkoxy, C₂-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆-alkoxy, formyloxy, C₁-C₆alkylcarbonyloxy, C₁-C₆alkylcarbonyl, C₁-C₆alkylthio, C₁-C₆alkylsulfinyl, C₁-C₆alkylsulfonyl, C₁-C₆haloalkylthio, C₁-C₆haloalkylsulfinyl or C₁-C₆halo-alkylsulfonyl, C₁-C₆alkylthiocarbonyl, C₁-C₆alkylamino, di(C₁-C₆alkyl)amino, C₁-C₄alkylsulfonyloxy, C₁-C₄alkylcarbonylamino, N(C₁-C₄alkyl)-C₁-C₄alkylcarbonylamino, C₁-C₄alkoxycarbonylamino, N(C₁-C₄alkyl)-C₁-C₄alkoxycarbonylamino, C₁-C₄alkylsulfonylamino, N(C₁-C₄alkyl)-C₁-C₄alkylsulfonylamino, OSO₂—C₁-C₄-alkyl, rhodano, tri(C₁-C₄alkyl)silyl, C₁-C₄alkyl(C₁-C₄alkoxy)phosphino or di(C₁-C₄alkoxy)phosphono;

or X₆ is a three- to ten-membered mono- or bicyclic ring system, which may be aromatic, saturated or partially saturated and can contain from 1 to 4 hetero atoms selected from the group consisting of nitrogen, oxygen, sulfur, —S(O)—, —S(O)₂—, —N(Ra₂₆)—, —C(O)— and C(═NORa₇), and each ring system can contain not more than two oxygen atoms and not more than two sulfur atoms, and the ring system can itself be mono- or poly-substituted by C₁-C₆alkyl, C₁-C₆haloalkyl, C₂-C₆alkenyl, C₂-C₆haloalkenyl, C₂-C₆alkynyl, C₂-C₆haloalkynyl, hydroxy, C₁-C₆alkoxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₄alkylaminosulfonyl, di(C₁-C₄alkyl)aminosulfonyl, amino, C₁-C₄alkylamino, di(C₁-C₄alkyl)amino, halogen, cyano, nitro, phenyl, phenoxy, phenylthio, benzyloxy and/or by benzylthio, it being possible for phenyl groups in turn to be substituted on the phenyl ring by C₁-C₃alkyl, C₁-C₃haloalkyl, C₁-C₃alkoxy, C₁-C₃haloalkoxy, C₁-C₃alkylsulfonyl C₁-C₃haloalkyl-sulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;

R₃ is hydrogen, C₁-C₄alkyl, C₁-C₄haloalkyl, C₁-C₂alkoxy-C₁-C₂alkyl or phenyl, which in turn can be mono- or poly-substituted by C₁-C₃alkyl, C₁-C₃haloalkyl, C₁-C₃alkoxy, C₁-C₃haloalkoxy, C₁-C₃alkylsulfonyl C₁-C₃haloalkylsulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;

Ra₂₁ is hydrogen, C₁-C₄alkyl or C₁-C₂alkoxy-C₁-C₂alkyl;

Ra₂₂ is hydrogen, C₁-C₄alkyl or phenyl, which may be mono- or poly-substituted by C₁-C₃alkyl, C₁-C₃haloalkyl, C₁-C₃alkoxy, C₁-C₃haloalkoxy, C₁-C₃alkylsulfonyl C₁-C₃haloalkyl-sulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;

Ra₂₃ is hydrogen, formyl, C₁-C₄alkyl, C₁-C₄alkylcarbonyl, C₁-C₄haloalkylcarbonyl or C₁-C₄ alkoxycarbonyl;

Ra₂₅ is C₁-C₄alkyl, or is benzyl which can be mono- or polysubstituted by C₁-C₃alkyl, C₁-C₃haloalkyl, C₁-C₃alkoxy, C₁-C₃haloalkoxy, C₁-C₃alkylsulfonyl C₁-C₃haloalkylsulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;

Ra₂₄ and Ra₂₆ independently from each other, are hydrogen, C₁-C₄alkyl, C₁-C₄alkylthiocarbonyl, C₁-C₄alkoxycarbonyl, C₁-C₄alkylcarbonyl, C₃-C₄cycloalkylcarbonyl, phenylcarbonyl or phenyl, it being possible for the phenyl groups in turn to be mono- or polysubstituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, C₁-C₄alkylamino, di-C₁-C₄alkylamino, C₁-C₄alkyl-S, C₁-C₄alkyl-S(O), C₁-C₄alkyl-SO₂, C₁-C₄alkyl-S(O)₂O, C₁-C₄haloalkyl-S, C₁-C₄haloalkyl-S(O), C₁-C₄haloalkyl-SO₂, C₁-C₄haloalkyl-S(O)₂O, C₁-C₄alkyl-S(O)₂NH, C₁-C₄alkyl-S(O)₂N(C₁-C₄alkyl), halogen, nitro or by cyano;

R₄ is hydrogen, hydroxy, halogen, cyano, C₁-C₆-alkyl, C₁-C₆-haloalkyl, C₂-C₆-alkenyl, C₂-C₆-haloalkenyl, C₂-C₆-alkinyl, C₂-C₆-haloalkinyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆-haloalkylsulfinyl, C₁-C₆-haloalkylsulfonyl, C₁-C₆alkylaminosulfonyl, di-C₂-C₆alkylaminosulfonyl, C₁-C₆-alkylsulfonyloxy, amino, C₁-C₄alkylsulfonylamino, N(C₁-C₄alkyl)-C₁-C₄alkylsulfonylamino, nitro, triazolyl, furyl or phenyl, it being possible for phenyl in turn to be mono- or polysubstituted by C₁-C₃alkyl, C₁-C₃haloalkyl, C₁-C₃alkoxy, C₁-C₃haloalkoxy, C₁-C₃-alkylsulfonyl C₁-C₃haloalkylsulfonyl, aminosulfonyl, C₁-C₂alkylaminosulfonyl, di(C₁-C₂alkyl)-aminosulfonyl, di(C₁-C₄alkyl)amino, C₁-C₄alkoxycarbonyl, halogen, cyano or nitro;

R₅ is hydrogen, halogen, C₁-C₃alkyl, C₁-C₃haloalkyl or C₁-C₃alkoxy; or if R₅ is bound to the meta-position with regard to the carbonyl group and is hydrogen, the ortho-position with regard to the carbonyl group can be additionally cyano;

Q is a group Q₁

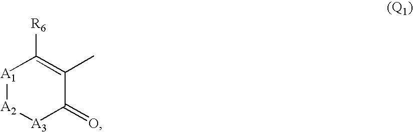

wherein

A₁ is C(R₁₁R₁₂) or NR₁₃;

A₂ is C(R₁₄R₁₅)ₘ, C(O), oxygen, NR₁₆ or S(O)_{q};

A₃ is C(R₁₇R₁₈) or NR₁₉;

with the proviso that A₂ is other than S(O)_{q} when A₁ is NR₁₃ and/or A₃ is NR₁₉;

R₆ is hydroxy, O⁻M⁺, wherein M⁺ is a metal cation or an ammonium cation; halogen or S(O)ₙR₉, wherein

m is 1 or 2;

q, n and k are each independently of the others 0, 1 or 2;

R₉ is C₁-C₁₂alkyl, C₂-C₁₂alkenyl, C₂-C₁₂alkynyl, C₃-C₁₂allenyl, C₃-C₁₂cycloalkyl, C₅-C₁₂cyclo-alkenyl, R₁₀-C₁-C₁₂alkylene or R₁₀-C₂-C₁₂alkenylene, wherein alkylene or alkenylene may be interrupted by —O—, —S(O)ₖ— and/or —C(O)— and can be mono- or poly-substituted by hydroxy, halogen, C₁-C₆alkyl, C₁-C₆alkoxy, C₁-C₆alkylthio, C₁-C₆alkylsulfinyl, C₁-C₆alkylsulfonyl, cyano, carbamoyl, carboxy, C₁-C₄alkoxycarbonyl or phenyl; it being possible for phenyl to be substituted by halogen, C₁-C₃alkyl, C₁-C₃haloalkyl, hydroxy, C₁-C₃alkoxy, C₁-C₃haloalkoxy, cyano or nitro; or

R₉ is phenyl or heteroaryl, each of which may be mono-, di- or tri-substituted by halogen, C₁-C₃alkyl, C₁-C₃haloalkyl, hydroxy, C₁-C₃alkoxy, C₁-C₃haloalkoxy, cyano or nitro;

R₁₀ is halogen, cyano, rhodano, hydroxy, C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, C₂-C₆alkenylthio, C₂-C₆alkynylthio, C₁-C₆alkylsulfonyloxy, phenylsulfonyloxy, C₁-C₆alkylcarbonyloxy, benzoyloxy, C₁-C₄alkoxy-carbonyloxy, C₁-C₆alkylcarbonyl, C₁-C₄alkoxycarbonyl, benzoyl, aminocarbonyl, C₁-C₄alkyl-aminocarbonyl, C₃-C₆cycloalkyl, phenyl, phenoxy, phenylthio, phenylsulfinyl or phenyl-sulfonyl; it being possible for the phenyl-containing groups in turn to be mono- or polysubstituted by halogen, C₁-C₃alkyl, C₁-C₃haloalkyl, hydroxy, C₁-C₃alkoxy, C₁-C₃haloalkoxy, cyano or nitro;

R₁₁ and R₁₇ are each independently of the other hydrogen, C₁-C₄alkyl, C₂-C₄alkenyl, C₂-C₄alkynyl, C₁-C₄alkylthio, C₁-C₄alkylsulfinyl, C₁-C₄alkylsulfonyl, C₁-C₄alkoxycarbonyl, hydroxy, C₁-C₄alkoxy, C₃-C₄alkenyloxy, C₃-C₄alkynyloxy, hydroxy-C₁-C₄alkyl, C₁-C₄alkyl-sulfonyloxy-C₁-C₄alkyl, halogen, cyano or nitro;

or, when A₂ is C(R₁₄R₁₅)ₘ, R₁₇ together with R₁₁ forms a direct bond, or a C₁-C₃alkylene or an ethenylene bridge;

R₁₂ and R₁₈ are each independently of the other hydrogen, C₁-C₄alkyl or C₁-C₄alkylthio, C₁-C₄alkylsulfinyl or C₁-C₄alkylsulfonyl;

or R₁₂ together with R₁₁, and/or R₁₈ together with R₁₇ form a C₂-C₅alkylene chain which can be interrupted by —O—, —C(O)— or —S(O)ₜ—;

t is 0, 1 or 2;

R₁₃ and R₁₉ are each independently of the other hydrogen, C₁-C₄alkyl, C₁-C₄haloalkyl, C₃-C₄alkenyl, C₃-C₄alkynyl or C₁-C₄alkoxy;

R₁₄ is hydrogen, hydroxy, C₁-C₄alkyl, C₁-C₄haloalkyl, C₁-C₃hydroxyalkyl, C₁-C₄alkoxy-C₁-C₃-alkyl, C₁-C₄alkylthio-C₁-C₃alkyl, C₁-C₄alkylcarbonyloxy-C₁-C₃alkyl, C₁-C₄alkylsulfonyloxy-C₁-C₃alkyl, tosyloxy-C₁-C₃alkyl, di(C₁-C₄alkoxy)-C₁-C₃alkyl, C₁-C₄alkoxycarbonyl, C₃-C₅-oxacycloalkyl, C₃-C₅thiacycloalkyl, C₃-C₄dioxacycloalkyl, C₃-C₄dithiacycloalkyl, C₃-C₄oxa-thiacycloalkyl, formyl, C₁-C₄alkoxyiminomethyl, carbamoyl, C₁-C₄alkylaminocarbonyl or di-(C₁-C₄alkyl)aminocarbonyl;

or R₁₄ together with R₁₁, R₁₂, R₁₃, R₁₇, R₁₈, R₁₉ or, when m is 2, also together with R₁₅, forms a direct bond or a C₁-C₄alkylene bridge;

R₁₅ is hydrogen, C₁-C₃alkyl or C₁-C₃haloalkyl;

R₁₆ is hydrogen, C₁-C₃alkyl, C₁-C₃haloalkyl, C₁-C₄alkoxycarbonyl, C₁-C₄alkylcarbonyl or N,N-di(C₁-C₄alkyl)aminocarbonyl; or

Q is a group Q₂

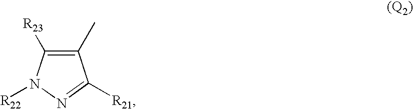

wherein

R₂₁ and R₂₂ are hydrogen or C₁-C₄alkyl;

R₂₃ is hydroxy, O⁻M⁺, wherein M⁺ is an alkali metal cation or ammonium cation; or is halogen, C₁-C₁₂alkylsulfonyloxy, C₁-C₁₂alkylthio, C₁-C₁₂alkylsulfinyl, C₁-C₁₂alkylsulfonyl, C₁-C₁₂halo-alkylthio, C₁-C₁₂haloalkylsulfinyl, C₁-C₁₂haloalkylsulfonyl, C₁-C₆alkoxy-C₁-C₆alkylthio, C₁-C₆-alkoxy-C₁-C₆alkylsulfinyl, C₁-C₆alkoxy-C₁-C₆alkylsulfonyl, C₃-C₁₂alkenylthio, C₃-C₁₂alkenyl-sulfinyl, C₃-C₁₂alkenylsulfonyl, C₃-C₁₂alkynylthio, C₃-C₁₂alkynylsulfinyl, C₃-C₁₂alkynylsulfonyl, C₁-C₄alkoxycarbonyl-C₁-C₄alkylthio, C₁-C₄alkoxycarbonyl-C₁-C₄alkylsulfinyl, C₁-C₄alkoxy-carbonyl-C₁-C₄alkylsulfonyl, benzyloxy or phenylcarbonylmethoxy; it being possible for the phenyl-containing groups to be mono- or polysubstituted by halogen, C₁-C₃alkyl, C₁-C₃haloalkyl, hydroxy, C₁-C₃alkoxy, C₁-C₃haloalkoxy, cyano or nitro; or

Q is a group Q₃

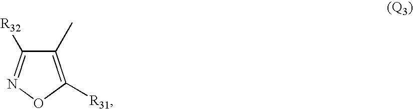

wherein

R₃₁ is C₁-C₆alkyl, C₁-C₆haloalkyl, C₃-C₆cycloalkyl or halo-substituted C₃-C₆cycloalkyl;

R₃₂ is hydrogen, C₁-C₄alkoxycarbonyl, carboxy or a group S(O)ₛR₃₃;

R₃₃ is C₁-C₆alkyl or C₁-C₃alkylene, which can be substituted by halogen, C₁-C₃alkoxy,

C₂-C₃alkenyl or by C₂-C₃alkynyl; and

s is 0, 1 or 2; or

Q is a group Q₄

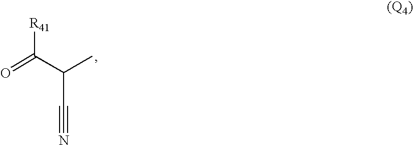

wherein

R₄₁ is C₁-C₆alkyl, C₁-C₆haloalkyl, C₃-C₆cycloalkyl or halo-substituted C₃-C₆cycloalkyl;

and the agrochemically acceptable salts and all stereoisomers and tautomers of compounds of formula I.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl, heptyl, octyl, nonyl, decyl, undecyl and dodecyl and their branched isomers. Alkoxy, alkenyl and alkynyl radicals are derived from the alkyl radicals mentioned. The alkenyl and alkynyl groups can be mono- or polyunsaturated including allenyl and mixed alkenylalkynyl groups.

Halogen is generally fluorine, chlorine, bromine or iodine, preferably fluorine and chlorine. This also applies, correspondingly, to halogen in combination with other meanings, such as haloalkyl or halophenyl.

Haloalkyl groups preferably have a chain length of from 1 to 6 carbon atoms. Haloalkyl is, for example, fluoromethyl, difluoromethyl, trifluoromethyl, chloromethyl, dichloromethyl, trichloromethyl, 2,2,2-trifluoroethyl, 1-fluoroethyl, 2-fluoroethyl, 2-chloroethyl, 1,1-difluoro-ethyl, pentafluoroethyl, 1,1-difluoro-2,2,2-trichloroethyl, 2,2,3,3-tetrafluoroethyl, 2,2,2-trichloroethyl and heptafloropropyl; preferably dichloromethyl, difluorochloromethyl, difluoromethyl, trifluoromethyl, 1-fluoroethyl and 1,1-fluoroethyl.

Suitable haloalkenyl groups are alkenyl groups which are mono- or polysubstituted by halogen, halogen being fluorine, chlorine, bromine and iodine and in particular fluorine and chlorine, for example 2,2-difluorovinyl, 2,2-dichlorovinyl, 2,2-difluoro-1-methylvinyl, 3-fluoropropenyl, 3-chloropropenyl, 3-bromopropenyl, 2,3,3-trifluoropropenyl, 2,3,3-trichloropropenyl and 4,4,4-trifluorobut-2-en-1-yl. Among the alkenyl groups which are mono- or polysubstituted by halogen, preference is given to those having a chain length of from 2 to 5 carbon atoms.

Suitable haloalkynyl groups are, for example, alkynyl groups which are mono- or polysubstituted by halogen, halogen being bromine, iodine and in particular fluorine and chlorine, for example 3-fluoropropynyl, 3-chloropropynyl, 3-bromopropynyl, 3,3,3-trifluoro-propynyl and 4,4,4-trifluorobut-2-yn-1-yl. Among the alkynyl groups which are mono- or polysubstituted by halogen, preference is given to those having a chain length of from 2 to 5 carbon atoms.

Alkoxy groups preferably have a chain length of from 1 to 6 carbon atoms. Alkoxy is, for example, methoxy, ethoxy, propoxy, i-propoxy, n-butoxy, isobutoxy, sec-butoxy and tert-butoxy and also the isomeric pentyloxy and hexyloxy radicals; preferably methoxy and ethoxy.

Alkylcarbonyl is, for example, acetyl, propionyl, isopropylarbonyl, n-butylcarbonyl, isobutylcarbonyl, sec-butylcarbonyl, tert-butylcarbonyl or neopentylcarbonyl; preferably acetyl or propionyl.

Alkoxycarbonyl is, for example, methoxycarbonyl, ethoxycarbonyl, propoxycarbonyl, isopropoxycarbonyl, n-butoxycarbonyl, isobutoxycarbonyl, sec-butoxycarbonyl or tert-butoxycarbonyl; preferably methoxycarbonyl or ethoxycarbonyl.

Haloalkoxy groups preferably have a chain length of from 1 to 8 carbon atoms. Haloalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy and 2,2,2-trichloroethoxy; preferably difluoromethoxy, 2-chloroethoxy and trifluoromethoxy.

Alkylthio groups preferably have a chain length of from 1 to 8 carbon atoms. Alkylthio is, for example, methylthio, ethylthio, propylthio, isopropylthio, n-butylthio, isobutylthio, sec-butylthio or tert-butylthio, preferably methylthio and ethylthio.

Alkylsulfinyl is, for example, methylsulfinyl, ethylsulfinyl, propylsulfinyl, isopropylsulfinyl, n-butylsulfinyl, isobutylsulfinyl, sec-butylsulfinyl, tert-butylsulfinyl; preferably methylsulfinyl and ethylsulfinyl.

Alkylsulfonyl is, for example, methylsulfonyl, ethylsulfonyl, propylsulfonyl, isopropylsulfonyl, n-butylsulfonyl, isobutylsulfonyl, sec-butylsulfonyl or tert-butylsulfonyl; preferably methylsulfonyl or ethylsulfonyl.

Alkoxyalkoxy groups preferably have a chain length of from 1 to 8 carbon atoms. Examples of alkoxyalkoxy groups are: methoxymethoxy, methoxyethoxy, methoxypropoxy, ethoxymethoxy, ethoxyethoxy, propoxymethoxy or butoxybutoxy, preferably methoxyethoxy.

Alkoxyalkyl groups preferably have a chain length of 1 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl, preferably methoxymethyl and ethoxymethyl.

Alkylthioalkyl groups preferably have from 1 to 8 carbon atoms. Alkylthioalkyl is, for example, methylthiomethyl, methylthioethyl, ethylthiomethyl, ethylthioethyl, n-propylthiomethyl, n-propylthioethyl, isopropylthiomethyl, isopropylthioethyl, butylthiomethyl, butylthioethyl or butylthiobutyl, preferably methylthiomethyl and ethylthiomethyl.

The cycloalkyl groups preferably have from 3 to 8 ring carbon atoms, for example cyclopropyl, cyclobutyl, cyclopentyl, cyclohexyl, cycloheptyl and cyclooctyl.

Alkylamino is, for example, methylamino, ethylamino, n-propylamino, isopropylamino or the isomers of butylamine. Dialkylamino is, for example, dimethylamino, methylethylamino, diethylamino, n-propylmethylamino, di-butylamino and di-isopropylamino. Preference is given to alkylamino and dialkylamino groups—including as a component of (N-alkyl)sulfonylamino and N-(alkylamino)sulfonyl groups, such as (N,N-dimethyl)sulfonylamino and N,N-(dimethyl-amino)sulfonyl—each having a chain length of from 1 to 4 carbon atoms.

Phenyl, including as a component of a substituent such as phenoxy, benzyl, benzyloxy, benzoyl, phenylthio, phenylalkyl, phenoxyalkyl, may be in substituted form. The substituents may in that case be in the ortho-, meta- and/or para-position(s). Preferred substituent positions are the ortho- and para-positions relative to the ring linkage site. The phenyl groups are preferably unsubstituted or mono- or di-substituted, especially unsubstituted or mono-substituted.

Heteroaryl is preferably pyridinyl, pyrimidinyl, triazinyl, triazolyl, thienyl, thiazolyl, oxazolyl or isoxazolyl.

In the context of the present invention, the term “mono- or poly-substituted” is generally to be understood as meaning mono- to penta-substituted, especially mono-, di- and trisubstituted.

According to the present invention, a three- to ten-membered monocyclic or fused bicyclic ring system which may be aromatic, partially saturated or fully saturated is, depending of the number of ring members, for example, selected from the group consisting of

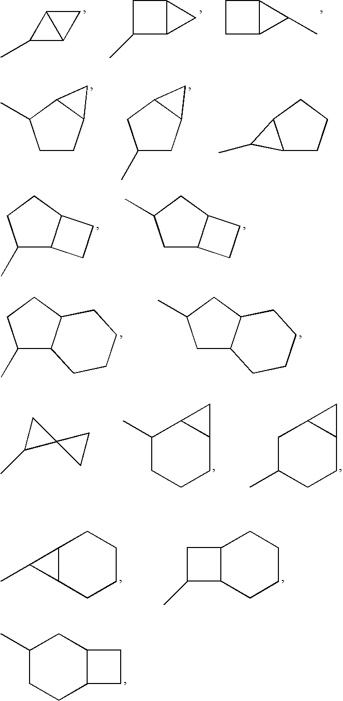

cyclopropyl, cyclobutyl, cyclopentyl, cyclohexyl, cyclohexenyl, where said cycloalkylgroups for their part may be preferably unsubstituted or substituted by C₁-C₆alkyl or halogen, or is phenyl, benzyl, indenyl, naphthyl or the following heterocyclic groups: pyrrolyl; pyridyl; pyrazolyl; pyrimidyl; pyrazinyl; imidazolyl; thiadiazolyl; quinazolinyl; furyl; oxadiazolyl; indolyl; pyranyl; isobenzofuranyl; thienyl; naphthyridinyl; (1-methyl-1H-pyrazol-3-yl)-; (1-ethyl-1H-pyrazol-3-yl)-; (1-propyl-1H-pyrazol-3-yl)-; (1H-pyrazol-3-yl)-; (1,5-dimethyl-1H-pyrazol-3-yl)-; (4-chloro-1-methyl-1H-pyrazol-3-yl)-; (1H-pyrazol-1-yl)-; (3-methyl-1H-pyrazol-1-yl)-; (3,5-dimethyl-1H-pyrazol-1-yl)-; (3-isoxazolyl)-; (5-methyl-3-isoxazolyl)-; (3-methyl-5-isoxazolyl)-; (5-isoxazolyl)-; (1H-pyrrol-2-yl)-; (1-methyl-1H-pyrrol-2-yl)-; (1H-pyrrol-1-yl)-; (1-methyl-1H-pyrrol-3-yl)-; (2-furanyl)-; (5-methyl-2-furanyl)-; (3-furanyl)-; (5-methyl-2-thienyl)-; (2-thienyl)-; (3-thienyl)-; (1-methyl-1H-imidazol-2-yl)-; (1H-imidazol-2-yl)-; (1-methyl-1H-imidazol-4-yl)-; (1-methyl-1H-imidazol-5-yl)-; (4-methyl-2-oxazolyl)-; (5-methyl-2-oxazolyl)-; (2-oxazolyl)-; (2-methyl-5-oxazolyl)-; (2-methyl-4-oxazolyl)-; (4-methyl-2-thiazolyl)-; (5-methyl-2-thiazolyl)-; (2-thiazolyl)-; (2-methyl-5-thiazolyl)-; (2-methyl-4-thiazolyl)-; (3-methyl-4-isothiazolyl)-; (3-methyl-5-isothiazolyl)-; (5-methyl-3-isothiazolyl)-; (1-methyl-1H-1,2,3-triazol-4-yl)-; (2-methyl-2H-1,2,3-triazol-4-yl)-; (4-methyl-2H-1,2,3-triazol-2-yl)-; (1-methyl-1H-1,2,4-triazol-3-yl)-; (1,5-dimethyl-1H-1,2,4-triazol-3-yl)-; (3-methyl-1H-1,2,4-triazol-1-yl)-; (5-methyl-1H-1,2,4-triazol-1-yl)-; (4,5-dimethyl-4H-1,2,4-triazol-3-yl)-; (4-methyl-4H-1,2,4-triazol-3-yl)-; (4H-1,2,4-triazol-4-yl)-; (5-methyl-1,2,3-oxadiazol-4-yl)-; (1,2,3-oxadiazol-4-yl)-; (3-methyl-1,2,4-oxadiazol-5-yl)-; (5-methyl-1,2,4-oxadiazol-3-yl)-; (4-methyl-3-furazanyl)-; (3-furazanyl)-; (5-methyl-1,2,4-oxadiazol-2-yl)-; (5-methyl-1,2,3-thiadiazol-4-yl)-; (1,2,3-thiadiazol-4-yl)-; (3-methyl-1,2,4-thiadiazol-5-yl)-; (5-methyl-1,2,4-thiadiazol-3-yl)-; (4-methyl-1,2,5-thiadiazol-3-yl)-; (5-methyl-1,3,4-thiadiazol-2-yl)-; (1-methyl-1H-tetrazol-5-yl)-; (1H-tetrazol-5-yl)-; (5-methyl-1H-tetrazol-1-yl)-; (2-methyl-2H-tetrazol-5-yl)-; (2-ethyl-2H-tetrazol-5-yl)-; (5-methyl-2H-tetrazol-2-yl)-; (2H-tetrazol-2-yl)-; (2-pyridyl)-; (6-methyl-2-pyridyl)-; (4-pyridyl)-; (3-pyridyl)-; (6-methyl-3-pyridazinyl)-; (5-methyl-3-pyridazinyl)-; (3-pyridazinyl)-; (4,6-dimethyl-2-pyrimidinyl)-; (4-methyl-2-pyrimidinyl)-; (2-pyrimidinyl)-; (2-methyl-4-pyrimidinyl)-; (2-chloro-4-pyrimidinyl)-; (2,6-dimethyl-4-pyrimidinyl)-; (4-pyrimidinyl)-; (2-methyl-5-pyrimidinyl)-; (6-methyl-2-pyr-azinyl)-; (2-pyrazinyl)-; (4,6-dimethyl-1,3,5-triazin-2-yl)-; (4,6-dichloro-1,3,5-triazin-2-yl)-; (1,3,5-triazin-2-yl)-; (4-methyl-1,3,5-triazin-2-yl)-; (3-methyl-1,2,4-triazin-5-yl)-; (3-methyl-1,2,4-triazin-6-yl)-;

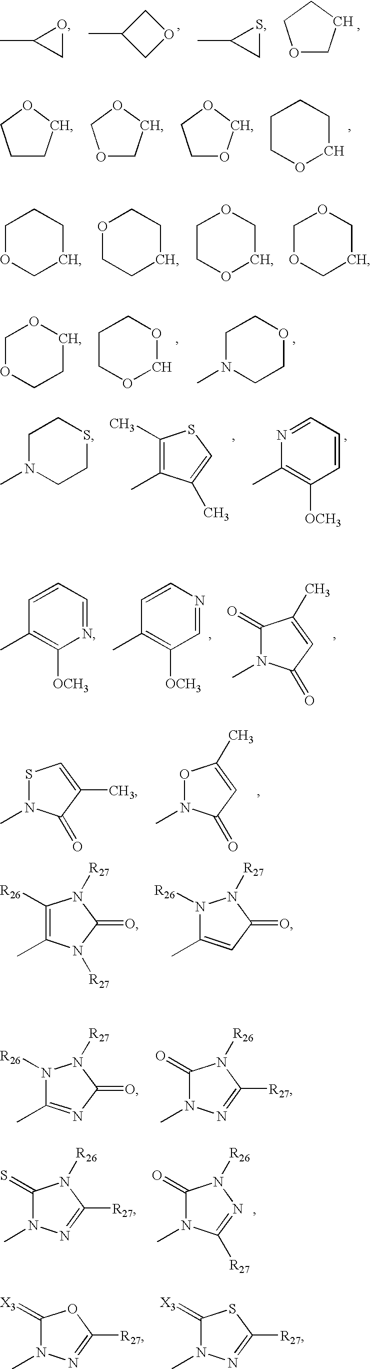

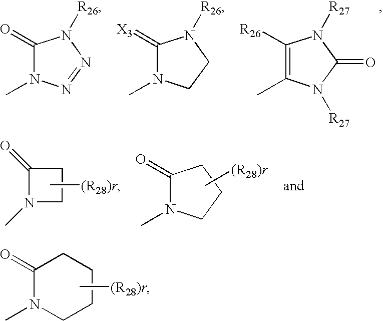

wherein each R₂₆ is methyl, each R₂₇ and each R₂₈ are independently hydrogen, C₁-C₃alkyl, C₁-C₃alkoxy, C₁-C₃alkylthio or trifluoromethyl, X₃ is oxygen or sulfur and r=1, 2, 3 or 4. Where no free valency is indicated in those definitions, for example as in

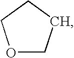

the linkage site is located at the carbon atom labelled “CH” or in a case such as, for example,

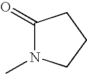

at the bonding site indicated at the bottom left.

The invention relates also to the salts which the compounds of formula I are able to form with amines, alkali metal and alkaline earth metal bases or quaternary ammonium bases, or with acid addition anions, where applicable per se due to basic groups. Among the alkali metal and alkaline earth metal hydroxides as salt formers, special mention should be made to the hydroxides of lithium, sodium, potassium, magnesium and calcium, but especially the hydroxides of sodium and potassium. In the context of the present invention, the alkali metal cation M⁺ (for example in the definition of R₆ and R₂₃) is preferably the sodium cation or the potassium cation. The compounds of formula I according to the invention also include the hydrates, which may be formed during the salt formation.

Examples of amines suitable for ammonium salt formation include ammonia as well as primary, secondary and tertiary C₁-C₁₈alkylamines, C₁-C₄hydroxyalkylamines and C₂-C₄-alkoxyalkylamines, for example methylamine, ethylamine, n-propylamine, isopropylamine, the four butylamine isomers, n-amylamine, isoamylamine, hexylamine, heptylamine, octyl-amine, nonylamine, decylamine, pentadecylamine, hexadecylamine, heptadecylamine, octadecylamine, methylethylamine, methylisopropylamine, methylhexylamine, methyl-nonylamine, methylpentadecylamine, methyloctadecylamine, ethylbutylamine, ethylheptyl-amine, ethyloctylamine, hexylheptylamine, hexyloctylamine, dimethylamine, diethylamine, di-n-propylamine, diisopropylamine, di-n-butylamine, di-n-amylamine, diisoamylamine, dihexyl-amine, diheptylamine, dioctylamine, ethanolamine, n-propanolamine, isopropanolamine, N,N-diethanolamine, N-ethylpropanolamine, N-butylethanolamine, allylamine, n-but-2-enyl-amine, n-pent-2-enylamine, 2,3-dimethylbut-2-enylamine, dibut-2-enylamine, n-hex-2-enyl-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylene-diamines, benzidines, naphthylamines and o-, m- and p-chloroanilines; but especially triethyl-amine, isopropylamine and diisopropylamine.

Preferred quaternary ammonium bases suitable for salt formation correspond, for example, to the formula [N⁺(RₐR_{b}R_{c}R_{d})⁻OH], wherein Rₐ, R_{b}, R_{c} and R_{d} are each independently of the others C₁-C₄alkyl. Further suitable tetraalkylammonium bases with other anions can be obtained, for example, by anion exchange reactions.

Examples of acid addition anions as salt performing agents are to mention especially the anions of hydrochloric acid, hydrobromic acid, hydroiodic acid, sulfuric acid, acetic acid, formic acid, trifluoracetic acid, oxalic acid and benzoic acid.

Depending upon the preparation process, the compounds of formula I may be obtained in various tautomeric forms, such as, for example, in Form A shown below or in Form B or in Form C, preference being given to Form A, as shown by way of example for compounds of formula IA wherein Q is a group Q₁ and the fused pyridine heterocycle is comprised of typus I-1 (definition vide infra). When R₆ is hydroxy, the structures of formula I can also be represented by the tautomeric Form D,

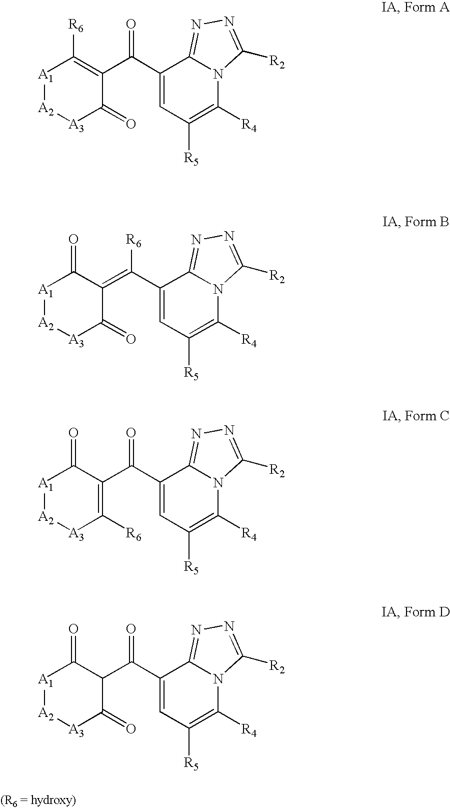

Compounds of formula I wherein Q is a group Q₂ or a group Q₄ can accordingly be present in the tautomeric forms A, B, C or D. Similarly, tautomeric forms also exist for compounds where R₁ or R₂ is hydroxy, thio and amino. When a C═N or C═C double bond is present in compounds of formula I, the compounds of formula I, when asymmetric, may be in the E form or the Z form. When a further asymmetric centre is present, for example an asymmetric carbon atom or a chiral —S(O)—, chiral <R>- or <S>-forms may occur. The present invention therefore relates also to all such stereoisomeric and tautomeric forms of the compound of formula I.

If X₁ respectively X₂ in the meaning of nitrogen, then the bond between the R₂ resp. R₁ bearing carbon atom CR₂ respectively CR₁ is a double bond. If X₁ respectively X₂ is a carbonyl, then the bond between the carbonyl C(O) and the R₅₁ respectively R₅₂ bearing nitrogen atom NR₅₁ respectively N₅₂ is a single bond.

In preferred compounds X₁ is nitrogen or CR₁ and X₂ is nitrogen if X₁ is CR₁; or is CR₂ if X₁ is nitrogen.

R₅ is preferably hydrogen, halogen, C₁-C₃alkyl, C₁-C₃haloalkyl or C₁-C₃alkoxy.

Preferred subgroups of the compounds of formula I are represented by the formulae I-1 to I-8:

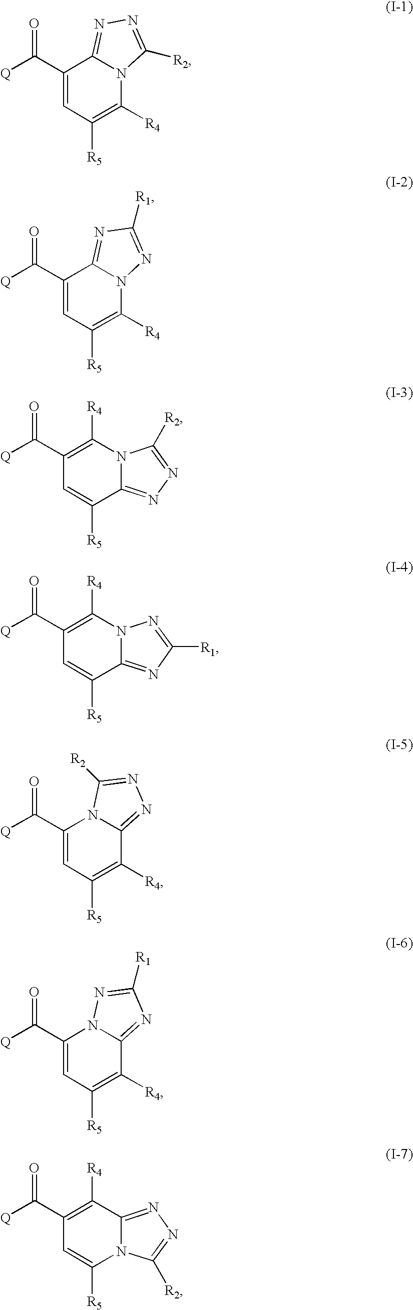

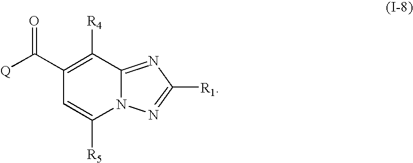

Especially preferred are the compounds of formula I-1,

wherein

Q is Q₁ or Q₂; preferably Q₁; wherein preferably

R₂ is hydrogen, halogen, or a group —X₆, —X₅—X₆ or —X₄—X₅—X₆;

X₄ is C₁-C₆alkylene, C₂-C₆alkenylene or C₂-C₆alkynylene chain, which can be mono-, di- or tri-substituted by halogen, hydroxy, C₁-C₆alkoxy, C₃-C₆cycloalkyloxy, C₁-C₆alkoxy-C₁-C₆alkoxy, C₁-C₆alkoxy-C₁-C₆alkoxy-C₁-C₆alkoxy or C₁-C₂alkylsulfonyloxy; or by a bivalent C₁-C₈alkylene group which may be interrupted by 1 to 2 oxygen atoms, sulphur or NRa₂₆, said bivalent C₁-C₈alkylene group can be substituted by halogen, hydroxy, amino, formyl, carboxy, nitro, cyano, mercapto, carbamoyl, C₁-C₆alkoxy, C₁-C₆alkoxycarbonyl, C₁-C₆-alkylaminocarbonyl, C₁-C₆-dialkylaminocarbonyl, C₂-C₆alkenyl, C₂-C₆haloalkenyl, C₂-C₆alkynyl, C₂-C₆haloalkynyl, C₂-C₆alkenyloxy, C₂-C₆alkynyloxy, C₁-C₆haloalkoxy, C₂-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆alkoxy, C₁-C₆alkylcarbonyl, C₁-C₆alkylthio, C₁-C₆alkylsulfinyl, C₁-C₆alkylsulfonyl, C₁-C₆haloalkylthio, C₁-C₆haloalkylsulfinyl or C₁-C₆halo-alkylsulfonyl, C₁-C₆alkylthiocarbonyl, C₁-C₆alkylamino, di(C₁-C₆alkyl)amino, C₁-C₄alkylsulfonyloxy, C₁-C₄alkylcarbonylamino, N(C₁-C₄alkyl)-C₁-C₄alkylcarbonylamino, C₁-C₄alkoxycarbonylamino, N(C₁-C₄alkyl)-C₁-C₄alkoxycarbonylamino, C₁-C₄alkylsulfonylamino, N(C₁-C₄alkyl)-C₁-C₄alkylsulfonylamino, OSO₂—₁-C₄-alkyl, rhodano, tri(C₁-C₄alkyl)silyl or di(C₁-C₄alkoxy)phosphono;

X₅ is oxygen, —OC(O)—, —OC(O)O—, —OC(O)N(R₃)—, OS(O)₂—, thio, sulfonyl, —C(O)O—, —C(O)—, —C(O)N(R₃)—, —N(R₃)C(O)—, —N(R₃)C(O)N(R₃)— or —N(R₃)SO₂N(R₃)—, preferably oxygene;

X₆ is C₁-C₆alkyl which may be mono-, di- or tri-substituted by halogen, hydroxy, amino, formyl, carboxy, nitro, cyano, mercapto, carbamoyl, C₁-C₆alkoxy, C₁-C₆alkoxycarbonyl, C₁-C₆-alkylaminocarbonyl, C₁-C₆-dialkylaminocarbonyl, C₂-C₆alkenyl, C₂-C₆haloalkenyl, C₂-C₆alkynyl, C₂-C₆haloalkynyl, C₂-C₆alkenyloxy, C₂-C₆alkynyloxy, C₁-C₆haloalkoxy, C₂-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆alkoxy, C₁-C₆alkylcarbonyl, C₁-C₆alkylthio, C₁-C₆alkylsulfinyl, C₁-C₆alkylsulfonyl, C₁-C₆haloalkylthio, C₁-C₆haloalkylsulfinyl or C₁-C₆halo-alkylsulfonyl, C₁-C₆alkylthiocarbonyl, C₁-C₆alkylamino, di(C₁-C₆alkyl)amino, C₁-C₄alkylsulfonyloxy, C₁-C₄alkylcarbonylamino, N(C₁-C₄alkyl)-C₁-C₄alkylcarbonylamino, C₁-C₄alkoxycarbonylamino, N(C₁-C₄alkyl)-C₁-C₄alkoxycarbonylamino, C₁-C₄alkylsulfonylamino, N(C₁-C₄alkyl)-C₁-C₄alkylsulfonylamino, OSO₂—C₁-C₄-alkyl, rhodano, tri(C₁-C₄alkyl)silyl or di(C₁-C₄alkoxy)phosphono;

or X₆ is a three- to ten-membered mono- or bicyclic ring system, which may be aromatic or saturated or partially saturated and may contain from 1 to 4 hetero atoms selected from aromatic nitrogen, oxygen, sulfur, —S(O)—, —S(O)₂—, —N(Ra₂₆)—, —C(O)— and/or C(═NORa₇), and each ring system may contain not more than two oxygen atoms and not more than two sulfur atoms, and the ring system can itself be mono-, di- or tri-substituted by C₁-C₆alkyl, C₁-C₆haloalkyl, C₂-C₆alkenyl, C₂-C₆haloalkenyl, C₂-C₆alkynyl, C₂-C₆haloalkynyl, hydroxy, C₁-C₆alkoxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di(C₁-C₂alkyl)aminosulfonyl, di(C₁-C₄alkyl)amino, halogen, cyano, nitro, phenyl, benzyloxy and/or by benzylthio, it being possible for phenyl groups in turn to be substituted on the phenyl ring by C₁-C₃alkyl, C₁-C₃haloalkyl, C₁-C₃alkoxy, C₁-C₃haloalkoxy, C₁-C₃alkylsulfonyl C₁-C₃haloalkylsulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;

R₄ is halogen, C₁-C₆-alkyl, C₁-C₆-haloalkyl, C₂-C₆-alkinyl, C₁-C₆-alkoxy, C₁-C₆-haloalkoxy, C₁-C₆-alkylthio, C₁-C₆-alkylsulfinyl, C₁-C₆-alkylsulfonyl, -C₆-haloalkylthio, C₁-C₆-haloalkylsulfinyl, triazolyl, furyl or phenyl, it being possible for phenyl in turn to be substituted by C₁-C₃alkyl, C₁-C₃haloalkyl, C₁-C₃alkoxy, -C₃haloalkoxy, C₁-C₃alkylsulfonyl C₁-C₃haloalkylsulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro; preferably C₁-C₃haloalkyl; and

R₅ is is hydrogen, halogen, C₁-C₃alkyl, C₁-C₃haloalkyl or C₁-C₃alkoxy; preferably hydrogen.

A further preferred subgroup of the compounds of formula I is represented by the group consisting of the compounds of formula I-1a, I-2a, I-1b, I-1c, I-1d, I-1e, I-2b, I-2d, I-5 (wherein Q is 5Me-CHD) and I-1j (wherein R₂₄ is C₁-C₆alkyl, preferably n-propyl and R₂₅ is hydrogen, or R₂₄ and R₂₅ together are C₂-C₆alkylen, preferably —CH₂CH₂—),

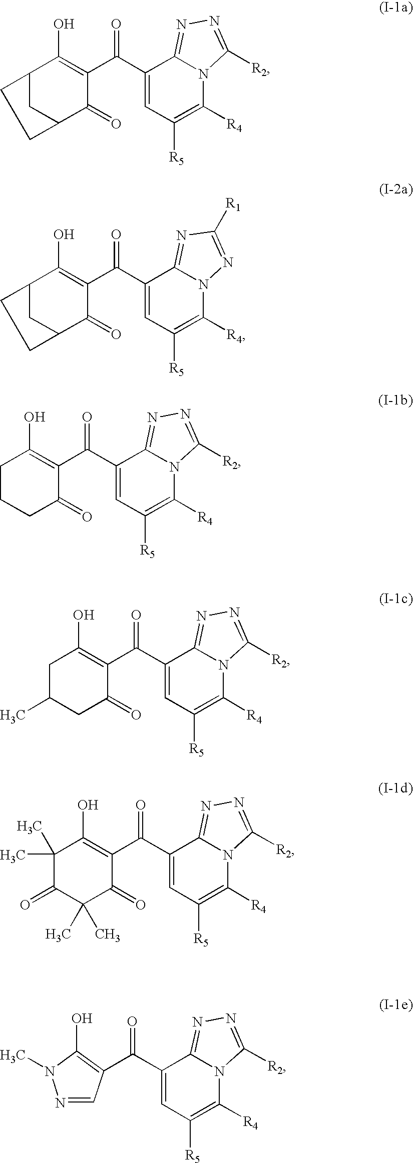

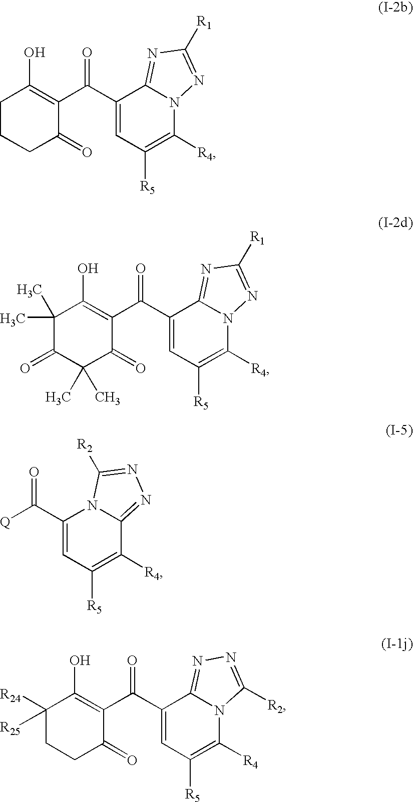

wherein

R₁ and R₂, independently from each other, are hydrogen, halogen, C₁-C₆alkyl, C₁-C₆haloalkyl, hydroxy-C₁-C₆alkyl, C₁-C₆alkoxy-C₁-C₆alkyl, C₁-C₆alkoxycarbonyl-C₁-C₆alkyl, phenoxycarbonyl-C₁-C₆alkyl, C₁-C₆alkylthio-C₁-C₆alkyl, C₁-C₆alkylsulfonyl-C₁-C₆alkyl, phenylsulfonyl-C₁-C₆alkyl, C₁-C₆sulfinyl-C₁-c₆alkyl, C₃-C₆cycloalkyl, C₂-C₆alkenyl, C₂-C₆alkinyl, (2-tetrahydrofuryl)-C₁-C₆alkoxy-C₁-C₆alkyl, amino, di-(C₁-C₆alkyl)amino, C₁-C₆alkylcarbonylamino, C₁-C₆alkoxy, C₂-C₆alkinyloxy, C₁-C₆alkoxy-C₁-C₆alkoxy, C₁-C₆haloalkoxy, phenyl, phenoxy, 4-chlorophenyl, mercapto, phenyl-C₁-C₆alkylthio, C₁-C₆alkylsulfonyl, di-(C₁-C₆alkyl)aminosulfonyl, phenylsulfonyl, Phenyl-C₁-C₆alkylsulfinyl, C₁-C₆alkylsulfinyl, phenylsulfinyl, phenylthio, 2-furyl, 2-pyridyl, 3-pyridyl or 4-pyridyl;

R₄ is C₁-C₆haloalkyl, C₁-C₆alkyl, cyano or triazolyl; and

R₅ is hydrogen, C₁-C₆alkyl, halogen or C₁-C₆alkoxy.

From this group, the following meanings of the substituents are especially preferred:

R₁ and R₂, independently from each other, are hydrogen, C₁-C₆alkyl, C₁-C₆cycloalkyl, C₁-C₆alkoxyalkyl or 4-chlorophenyl;

R₄ is C₁-C₆haloalkyl, preferably trifluoromethyl or difluoromethyl; and a

R₅ is hydrogen.

In a final preferred group of compounds X₁ is NR₅₁, if X₂ is C(O); or is C(O), if X₂ is NR₅₂; and

X₂ is NR₅₂, if X₁ is C(O); or is C(O), if X₁ is NR₅₁.

Preferably R₅₁_{_{—}} and R₅₂ independently from each other, are hydrogen, a group —X₆ or a group —X₄—X₅—X₆, wherein X₄, X₅ and X₆ are preferably as defined above.

The compounds of formula I can be prepared by means of processes known per se and are described for example in WO 00/15615, EP-A-0 316 491, EP-A-1 352 901, US 2003/0232984 and WO 02/16305 and as described below by way of example of compounds of formula IB

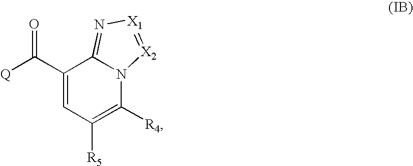

wherein Q, X₁, X₂, R₄ and R₅ are as defined previously.

In a preferred process, for example for the preparation of a compound of formula IB wherein Q is a group Q₁, Q₂ or Q₄, a compound of formula IIA

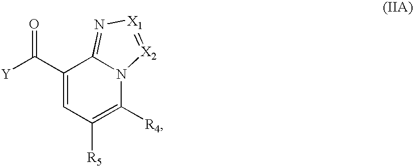

wherein X₁, X₂, R₄ and R₅ as described above, and Y is a leaving group, as for example fluorine, chlorine, p-nitro-phenoxy, cyano or the like, is reacted in the presence of a base with a keto compound of formula IIIa, IIIb or IIId

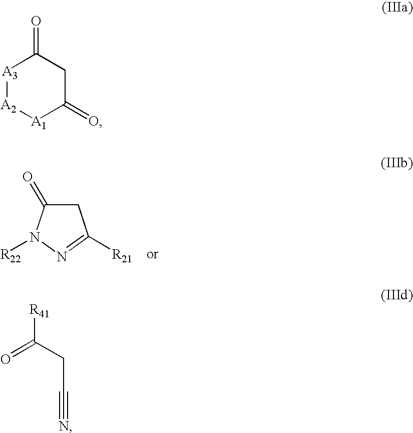

wherein A₁, A₂, A₃, R₂₁, R₂₂ and R₄₁ are as defined above, thus yielding the compound of formula IB directly in situ or yielding a compound of formula IVA

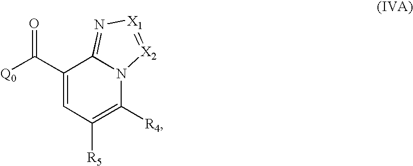

wherein X₁, X₂, R₄ and R₅ are as defined above and Q₀ is accordingly the group Q linked to oxygen, which compound, especially when Y is other then cyanide, as for example is chlorine, is then rearranged in the presence of an additional catalytic amount of cyanide releasing source, e.g. potassium cyanide, trimethylsilyl cyanide or acetone cyanohydrin, and in the presence of a base, e.g. triethylamine, to form a C—C-linked compound IB.

That process is illustrated by way of example with respect to compounds of formula IB wherein Q is a group Q₁, that is to say with respect to compounds of formula IBa, in Scheme 1.

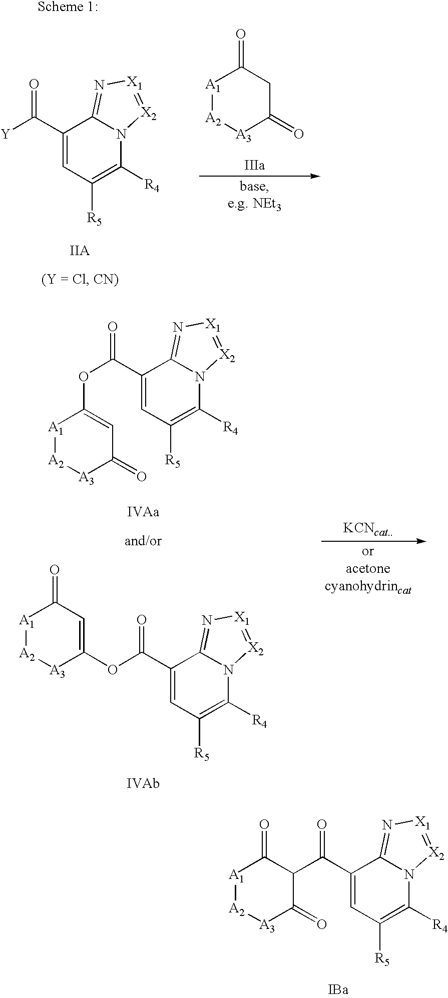

In a variant of that process, for example for the preparation of a compound of formula IB, wherein X₁, X₂, R₄ and R₅ are as defined above and Q is a group Q₁, Q₂ or Q₄, a compound of formula IIAd

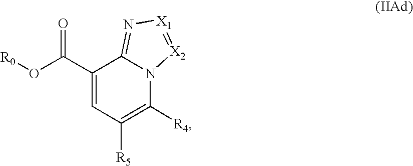

wherein X₁, X₂, R₄ and R₅ are as defined above and R₀ is hydrogen, is reacted with the aid of a coupling reagent, for example dicyclohexylcarbodiimide, (1-chloro-2-methyl-propenyl)-dimethylamine or 2-chloro-1-methylpyridinium iodide, in the presence of a base, e.g. triethylamine or Hünig base, with a keto compound of formula IIIa, IIIb or IIId, respectively,

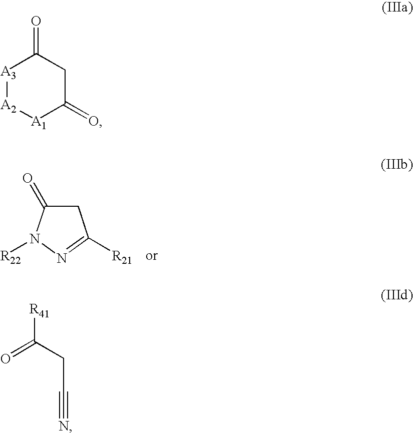

wherein A₁, A₂, A₃, R₂₁, R₂₂ and R₄₁ are as defined above, optionally via an intermediate of an activated ester of formula IIAe

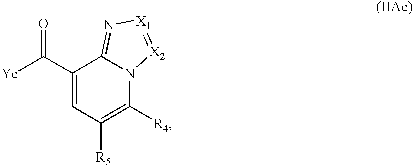

wherein X₁, X₂, R₄ and R₅ are as defined above and the meaning of Ye depends upon the coupling reagent used, to form a compound of formula IVA

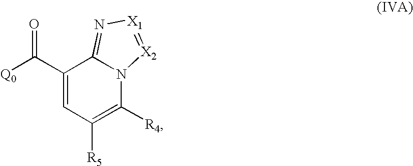

wherein X₁, X₂, R₄ and R₅ are as defined above and Q₀ is accordingly the group Q linked to oxygen, and that compound is then, after isolation in a second reaction step or directly in situ, rearranged in the presence of a base, e.g. triethylamine and a catalytic amount of cyanide ions, e.g. potassium cyanide or acetone cyanohydrin, or a catalytic amount of dimethylaminopyridine, to form a C—C-linked compound IB.

That process is illustrated by way of example with respect to compounds of formula IB wherein Q is a group Q₁, that is to say with respect to compounds of formula IAa, in Scheme 2.

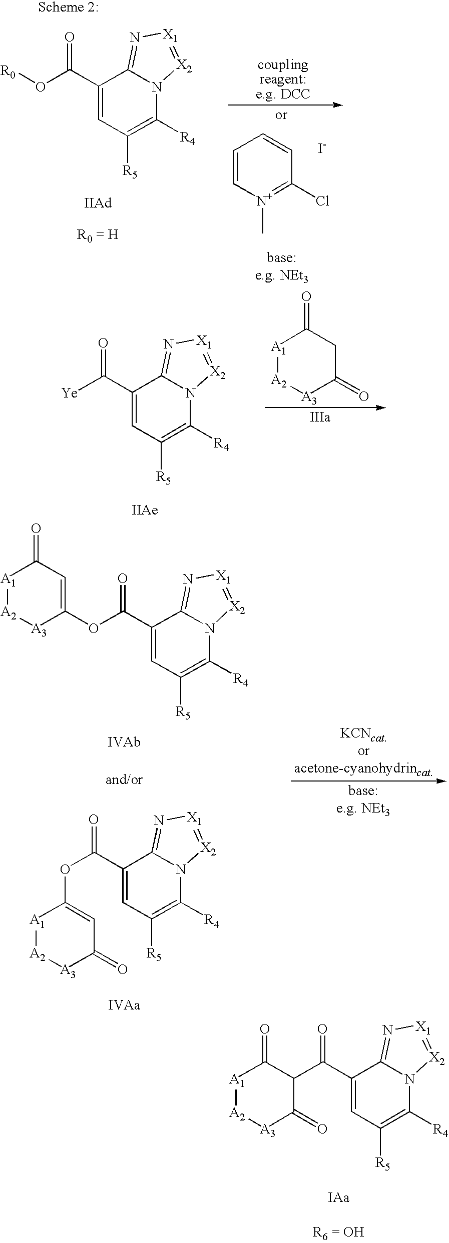

An important modification to the above mentioned process is using cyanide catalysis earlier in the sequence by adding the cyanide source to the active ester of formula IIAe simultaneously with the addition of the nucleophile. This procedure allows for direct C-coupling with the ambident nucleophilic partner IIIa in the highlighted case, enabling product formation without necessarily going through an O-intermediate. This modification is illustrated in scheme 2a

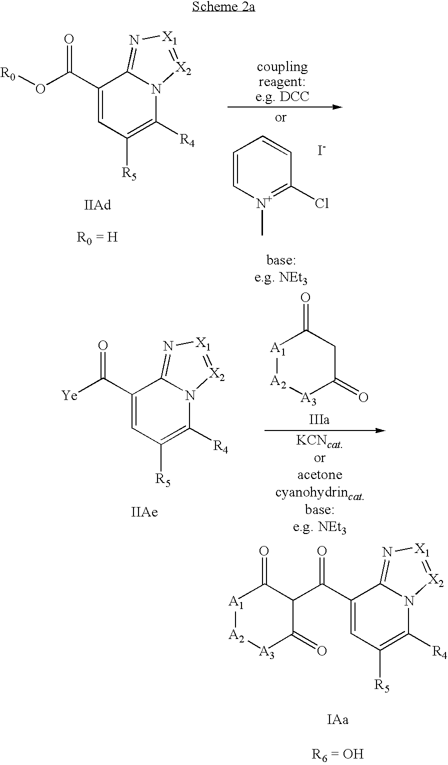

In a further process for the preparation of compounds of formula IB, a compound of formula VA

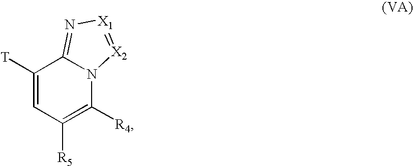

wherein X₁, X₂, R₄ and R₅ are as defined above and T is chlorine, bromine, iodine or trifluoromethanesulfonyloxy, is reacted under carbonylation conditions, as described, for example, in Tetrahedron Letters, 31, 2841, 1990 and in WO 02/16305, in the presence of noble metal catalysts and suitable phosphine ligands, e.g. Pd(PPh₃)₄ or Pd(PPh₃)₂Cl₂, and suitable bases, e.g. triethylamine, with a compound of formula III, for example of formula IIIa or IIIb

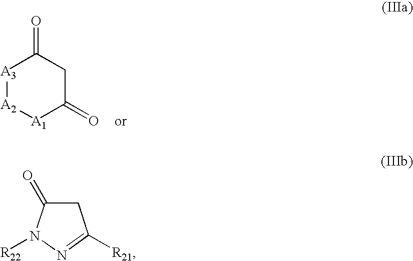

wherein A₁, A₂, A₃, R₂₁ and R₂₂ are as defined above, as illustrated in Scheme 3 for compounds of formula IAa wherein R₆ is hydroxy.

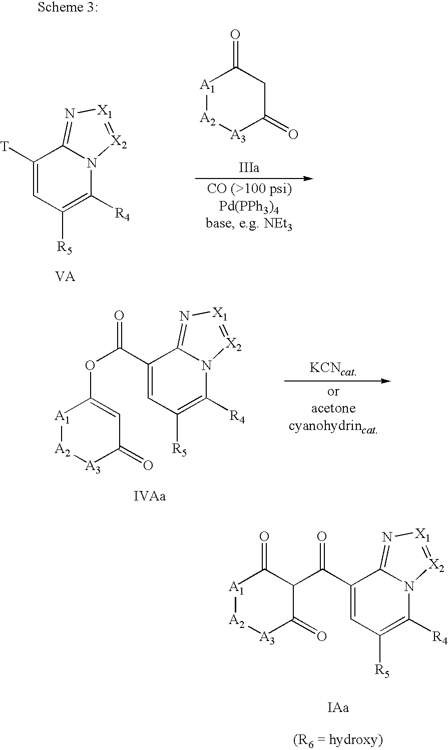

Compounds of formula IB

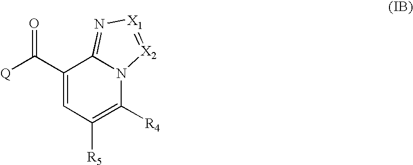

wherein Q, X₁, X₂, R₄ and R₅ are as defined above and Q is a group Q₃, that is to say compounds of formula IAc

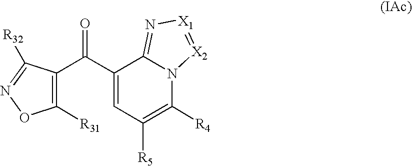

can likewise be prepared analogously to known procedures (for example following procedures described in WO 00/15615 and WO 01/94339). By way of example, a compound of formula IIA

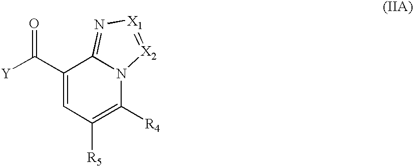

wherein X₁, X₂, R₄ and R₅ are as defined above and Y is chlorine is converted in a Claisen condensation with a ketocarboxylic acid salt of formula XIV

R₃₁C(O)CH₂COO⁻M⁺ (XIV)

or with a trialkyl silyl ester of formula XIVa

R₃₁C(O)CH₂COOSi(R′R″R′″)₃ (XIVa),

wherein R₃₁ is as defined above and M⁺ is a metal salt cation, e.g. Li⁺ or K⁺, and R′, R″, R′″ are a C₁-C₄alkyl group, e.g. methyl, into a compound of formula IIAa

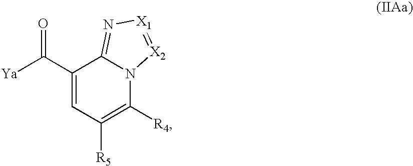

wherein X₁, X₂, R₄ and R₅ are as defined above and Ya is CH₂C(O)R₃₁, that compound is then treated in the presence of a base with carbon disulfide and an alkylating reagent of formula XV

R₃₃Y₂ (XV),

wherein R₃₃ is as defined for formula I and Y₂ is a leaving group, such as halogen or sulfonyloxy, and converted into a compound of formula IIAb

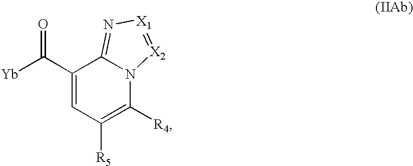

wherein X₁, X₂, R₄ and R₅ are as defined above and Yb is a group Yb

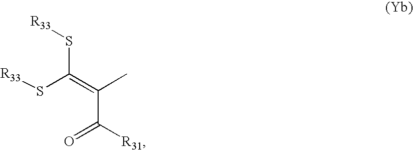

and then the compound of formula IIAb is cyclised with hydroxylamine hydrochloride and optionally in a solvent and in the presence of a base, for example sodium acetate, to form isomeric compounds of formula IAc and/or IAe, and the latter are then, when n is 1 or 2, oxidised with an oxidising agent, e.g. with a peracid, such as meta-chloroperbenzoic acid (m-CPBA) or peracetic acid, to form corresponding sulfoxides (n=1) or sulfones (n=2) of formula IAc

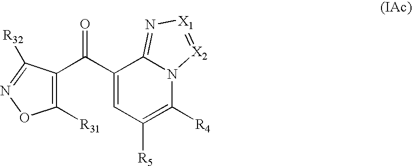

and IAe

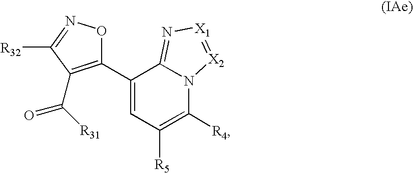

wherein X₁, X₂, R₄ and R₅ are as defined above and R₃₂ is a group S(O)ₛR₃₃. That process is illustrated in Scheme 4.

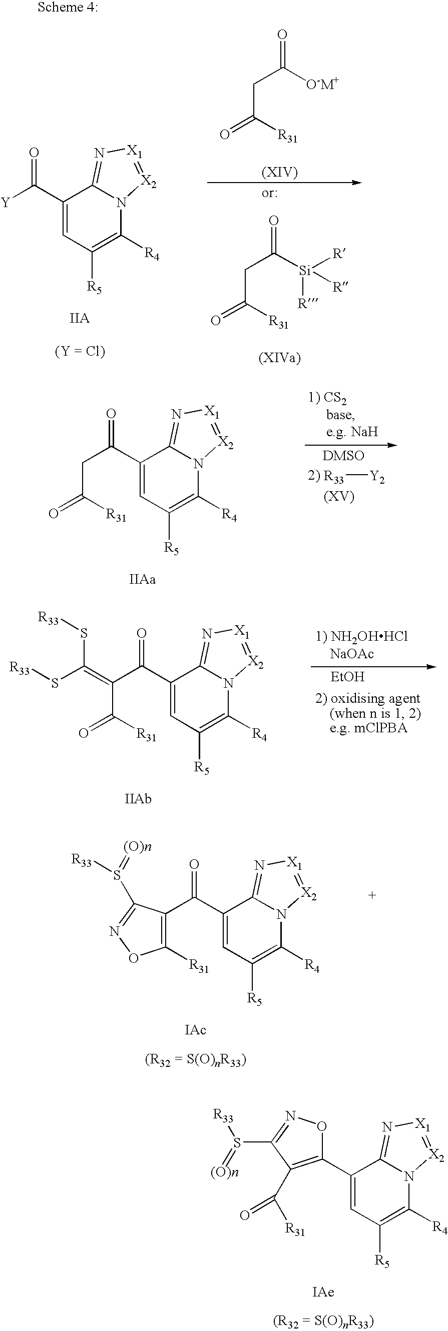

Compounds of formula IAc

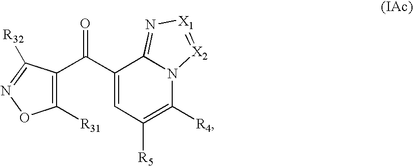

wherein X₁, X₂, R₄ and R₅ are as defined above and R₃₂ is hydrogen, C₁-C₄alkoxycarbonyl or carboxy, can likewise be prepared analogously to known procedures (e.g. analogously to the procedures described in WO 97/46530), for example as follows: a compound of formula IIAa

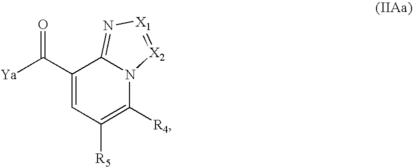

wherein X₁, X₂, R₄ and R₅ are as defined above and Ya is CH₂C(O)R₃₁, is converted in the presence of a base with an ortho ester of formula XVI

R₃₂C(OR″)₂Y₃ (XVI)

or with a cyanic acid ester of formula XVII

R′″OC(O)CN (XVII),

wherein R₃₂ is hydrogen, Y₃ is a leaving group, such as C₁-C₄alkoxy or di(C₁-C₄alkyl)amino, and R″ and R′″ are C₁-C₄alkoxy, into a compound of formula IIAc

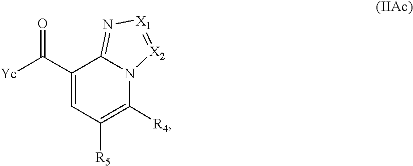

wherein X₁, X₂, R₄ and R₅ are as defined above and Yc is a group Yca

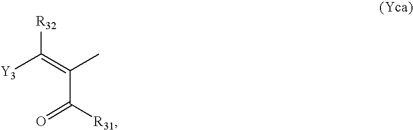

wherein R₃₁ is as defined above and R₃₂ is hydrogen or C₁-C₄alkoxycarbonyl and Y₃ is a leaving group, such as C₁-C₄alkoxy or di(C₁-C₄alkyl)amino, or hydroxy, and then the compound of formula IIAc is cyclised with hydroxylamine hydrochloride and optionally in a solvent and in the presence of a base, for example sodium acetate, to form isomeric compounds of formula IAc and/or IAe, and the latter are then, when R₃₂ is carboxyl or hydrogen, treated with a hydrolysing agent, e.g. with potassium hydroxide followed by a mineral acid, such as hydrochloric acid, to yield compounds of formula IAc

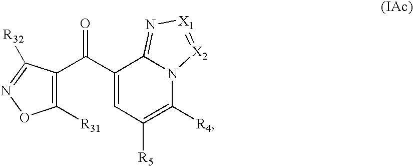

and/or IAe

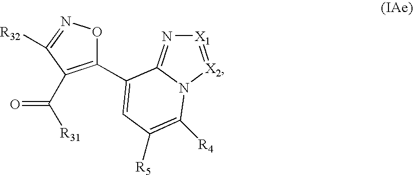

wherein X₁, X₂, R₄, R₅ and R₃₁ are as defined above and R₃₂ is hydrogen, C₁-C₄-alkoxycarbonyl or carboxy. That process is illustrated in Scheme 5.

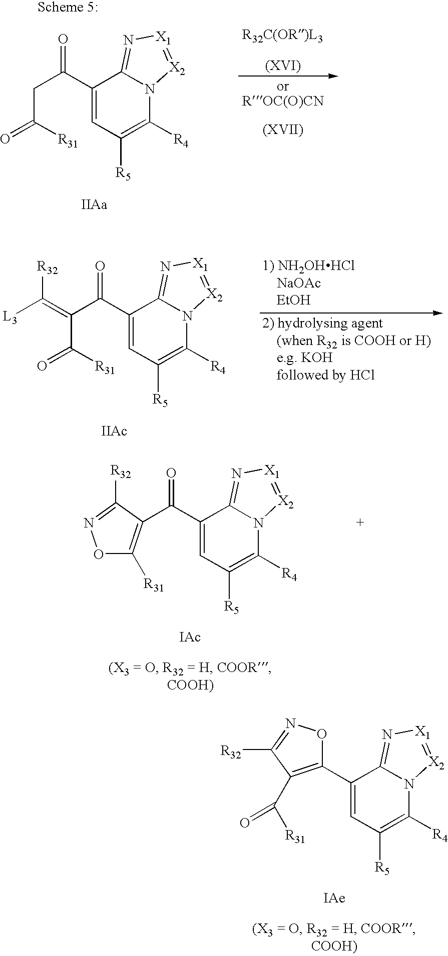

The isomeric compounds of formula IAc and IAe can be separated and purified, for example by means of column chromatography and a suitable eluant. In addition, compounds of formula IAe represent a sub-group of compounds of formula IB and accordingly the present invention relates likewise thereto.

Compounds of formula IB

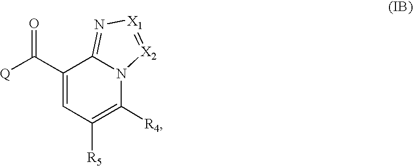

wherein X₁, X₂, R₄ and R₅ are as defined above and R₆ or R₂₃ in the group Q₁ or Q₂, as the case may be, is S(O)ₙR₉ can likewise be prepared in accordance with known procedures by reacting a compound of formula IB wherein X₁, X₂, R₄ and R₅ are as defined above and R₆ or R₂₃ in the group Q₁ or Q₂, respectively, is hydroxy, with a chlorinating agent, e.g. with oxalyl chloride, and then reacting the resulting compound of formula IB wherein X₁, X₂, R₄ and R₅ are as defined above and R₆ or R₂₃ in the group Q₁ or Q₂, respectively, is chlorine, with a thio compound of formula VI

HSR₉ (VI)

or with a salt of formula VIa

M⁺⁻SR₉ (VIa),

wherein R₉ is as defined above, and optionally with an additional base, e.g. triethylamine, sodium hydride, sodium hydrogen carbonate or potassium carbonate, and for the preparation of a compound of formula IB X₁, X₂, R₄ and R₅ are as defined above and R₆ or R₂₃ in the group Q₁ or Q₂, respectively, is S(O)ₙR₉ and n is 1 or 2, treating the resulting compound of formula IB wherein X₁, X₂, R₄ and R₅ are as defined above and R₆ or R₂₃ in the group Q₁ or Q₂, respectively, is SR₉, with an oxidising agent, e.g. sodium perbromate, sodium iodate, peracetic acid or m-chloroperbenzoic acid. That process sequence is illustrated in Scheme 6 using the example of compounds of formula IAa as defined above.

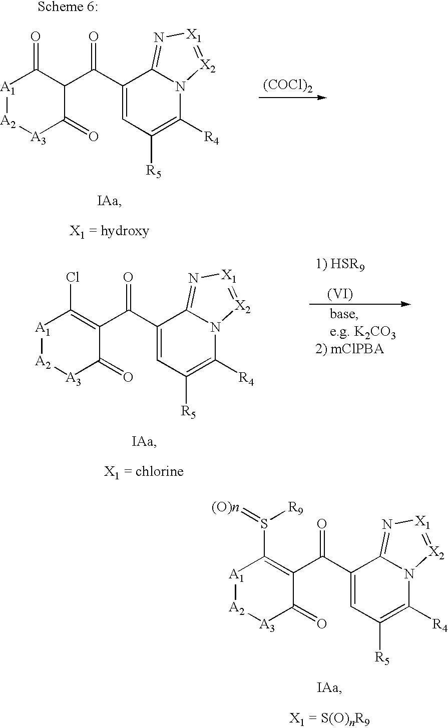

The compounds of formula IIA

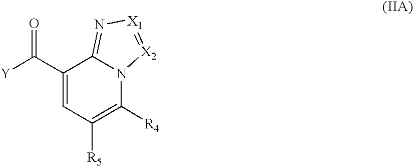

wherein X₁, X₂, R₄ and R₅ are as defined above and Y is a leaving group, as fluorine, chlorine, p-nitro-phenoxy, cyano or the like can be prepared by known methods from compounds of formula IIA wherein Y is hydroxy, C₁-C₄alkoxy, benzyloxy, phenoxy or allyloxy, that is to say from compounds of formula IIAd

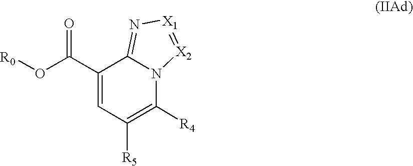

wherein X₁, X₂, R₄ and R₅ are as defined above and accordingly R₀ is hydrogene, C₁-C₄alkyl, benzyl, phenyl or allyl.

Methods to synthesize compounds of formula IIAd are known. An overview about existing synthetic routes is given by Jones & Sliskovic in Advances in Heterocylic Chemistry, 34, 79 (1983) and by Jones in Advances in Heterocylic Chemistry, 83, 1 (2002).

One method described by way of example is by reacting compound of formula VIIa, which are partially known and described for example in WO 2000/39094 and U.S. Pat. No. 5,235,060, with hydrazine, yielding hydrazino-pyridines of formula VIIIa, which in turn can be coupled with a range of compounds like eg carboxylic acid derivatives to yield compounds of formula IXa. Further treatment of cpds IXa with dehydration reagents like eg SOCl₂, POCl₃, etc. yields fused systems of the type of [4,3-a]triazolo-pyridines. Interestingly, and also described by Jones & Sliskovic, and depending on the nature of the substitution pattern of R₀, R₂, R₄, R₅ and the condition of hydrolysis of compounds of the formula Xa, wherein R₀ is different to hydrogen, the isomeric triazolo-pyridins additionally claimed in this patent, the [1,5-a]triazolo-pyridines of formula Xb can be obtained by just extending the reaction sequence, ie by rearrangent of cpds Xa through further treatment with hydroxide as described in J. Het. Chem. 1970, 7, 1019. Compounds Xa and Xb are subgroups of compounds of formula IIAd. And compounds of formula Xb are useful in the preparation of compounds of formula I-2. The overall synthetic sequence is illustrated in scheme 7 and highlights the general overall process to compounds of formula IIAd.

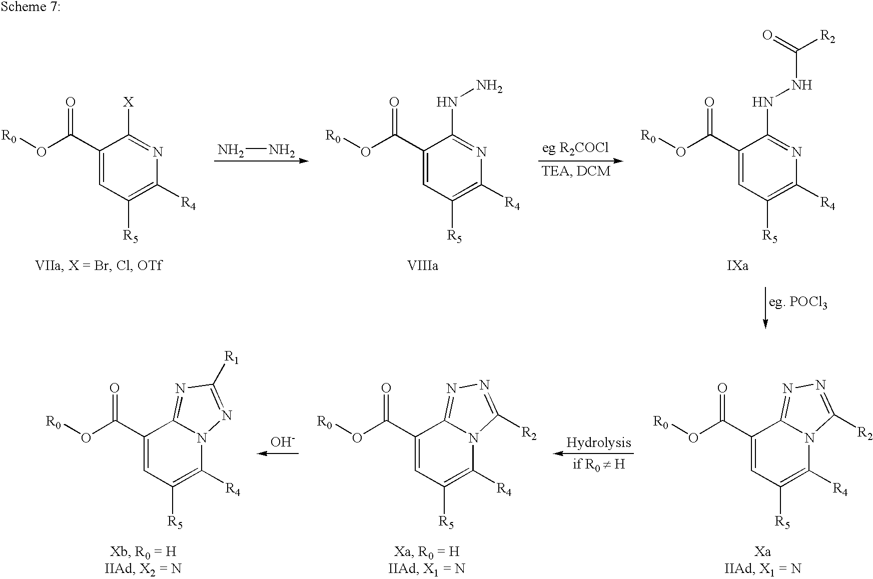

Similar to the method illustrated in scheme 7 intermediates of formula XA and XB

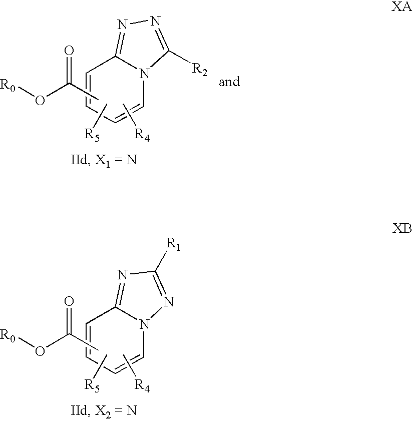

that are especially useful for the preparation of compounds of formula I-1 to I-8 can be prepared from intermediates of general formula VII,

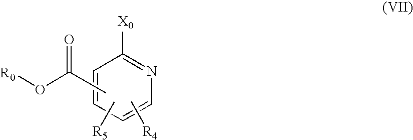

wherein X₀ is a leaving group, as eg fluorine, chlorine, bromine, triflate or the like.

Compounds of formula VII are known per se or can be prepared by the methods described for example in WO 00/15615, WO 2005/58830 and the citated reference above.

A plethora of methods to build up pyridine systems with suitable substituent patterns are known. The Handbook of Heterocyclic Chemistry, by A. R. Katrizky and A. F. Pozharskii gives an overview of the state-of-the-art of synthesis in this field. By way of example another reaction sequence leading to [4,3-a]triazolo-pyridines is mentioned, highlighting the great flexibility for alternative tactics in the order of bond formation. The key step of the reaction sequence, particularly suited to access CF₃-substituted pyridines, is described by e.g Cocco et al. in J. Heterocyclic Chemistry, 33, 1771 (1996). Treatment of cyano-ethylacetate with anhydrous HCl leads to the formation of the hydrochloride salt of the corresponding chloroimidate, which in turn is reacted with acylhydrazines to give amidrazones of formula XI. Further reaction with trifluoroacylenolether leads to pyridines of general formula XII which can be cyclized to fused systems with dehydration agents like e.g. POCl₃. The overall route is illustrated in Scheme 8.

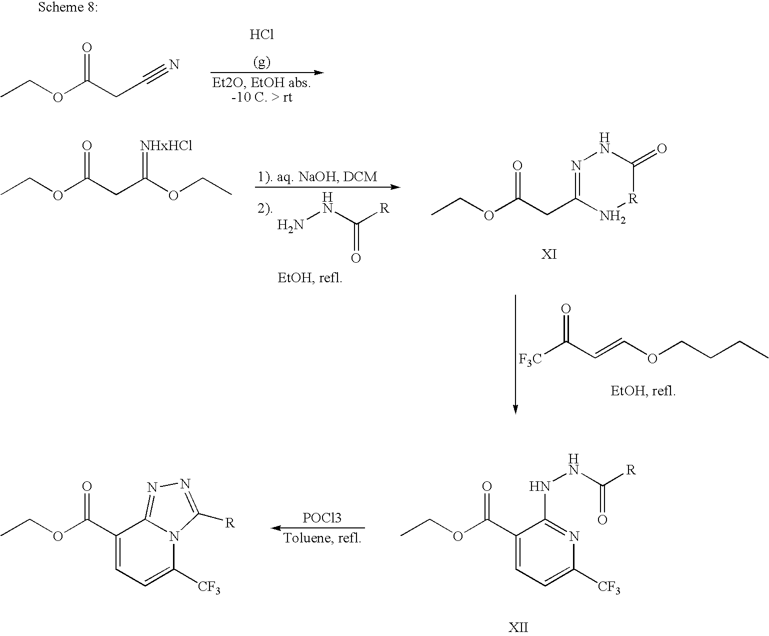

Beyond the synthetic strategy to introduce substituents R₅₁, R₅₂, R₁, R₂, R₄ and R₅ as part of the sequence leading to the construction of the triazolo-pyridine nucleus, there is always the possibility to modify substituents once the heterocyclic nucleus has been formed. Many methods for further manipulation or change of the nature of substituents R₅₁, R₅₂, R₁, R₂, R₄, R₅ are known to the one skilled in the art, some of them, but by no means exhaustive, are e.g described by Potts et. al. in J. Org. Chem, 31, 251-73.

The compounds of formulae IIA, IIAa, IIAb, IIAc, IIAd, IIAe, IVA, IVAa and IVAb are valuable intermediates in the preparation of compounds of formula IB wherein Q, X₁, X₂, R₄ and R₅ are as defined previously and accordingly the present invention relates also thereto.

All those intermediates according to the invention are represented by the general formula II

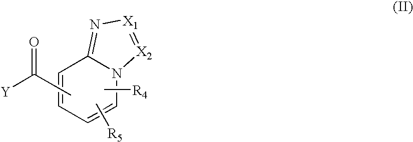

wherein Y is fluorine, chlorine, cyano, hydroxy, C₁-C₄alkoxy, allyloxy, benzyloxy, phenoxy, or benzyloxy, phenoxy substituted by C₁-C₄-alkyl, halogen, cyano, nitro, C₁-C₄-alkoxycarbonyl, C₁-C₃-alkylsulfinyl or C₁-C₃-alkylsulfonyl, or Y is a group

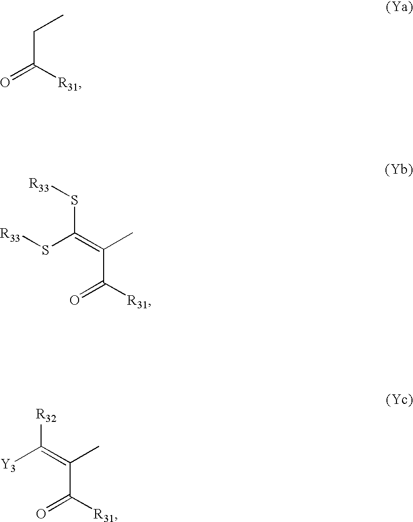

or a group Q₀, wherein Q₀ is accordingly a group Q linked to oxygen and Q, X₁, X₂, R₄ and R₅ are as defined above for formula 1.

Furthermore the compounds of the general formula IId

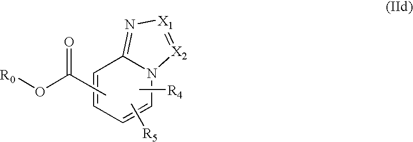

and in special formula IIAd, wherein X₁, X₂, R₄, R₅ and R₀ are as defined above can be prepared by known carbonylation processes per se from compounds of formula V

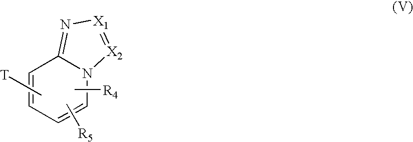

and in special IIAd from formula VA, wherein X₁, X₂, R₄, R₅ and T are defined as above, as for example described in Organic Process Research & Development (2001), 5(6), 572-574, U.S. Pat. No. 4,995,902 and U.S. Pat. No. 6,015,911.

This process is illustrated in scheme 9 for compounds of formula IIAd prepared form compounds of formula VA.

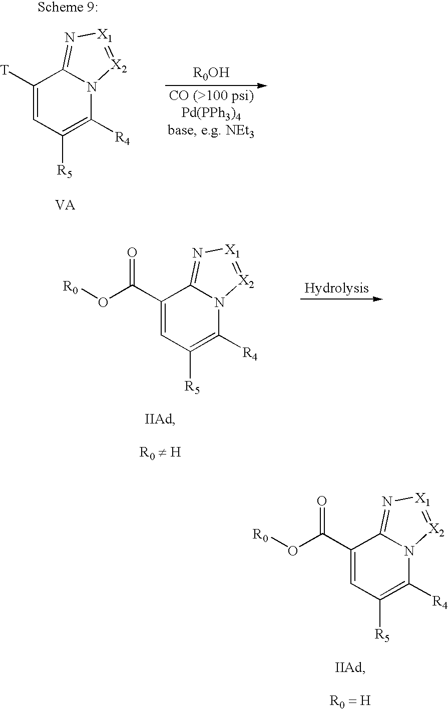

Compounds of formula IId,

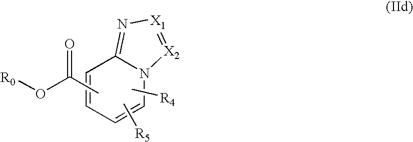

wherein R₀ is hydroxy and X₁, X₂, R₄ and R₅ are as defined for formula I with the proviso that R₄ is different from hydrogen if R₅ is hydrogen or chlorine are a further object of the present invention.

The compounds of formula IIIa, IIIb and IIId used as starting materials are known or can be prepared in accordance with generally described methods, e.g. as described in the references mentioned above. Alternatively, compounds of formula IIIa₀

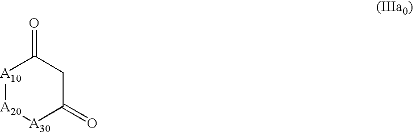

wherein

A₁₀ is C(R₁₁₀R₁₂₀);

A₂₀ is C(R₁₄₀R₁₅₀)ₘ;

A₃₀ is C(R₁₇₀R₁₈₀);

m is 1 or 2; and

R₁₁₀, R₁₂₀, R₁₄₀, R₁₅₀, R₁₇₀, R₁₈₀ are each independently of the other hydrogen or C₁-C₄alkyl;

or R₁₇₀ together with R₁₁₀ forms a C₁-C₃alkylene or an ethenylene bridge, can be prepared as described in WO 05/105718 and WO 05/105717. In this process, a compound of formula XVIII

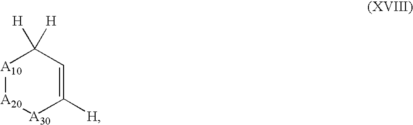

wherein A₁₀, A₂₀ and A₃₀ are as defined for formula IIIa₀, is in step a) reacted with a bromine or chlorine source to form a compound of formula XIX

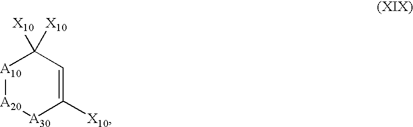

wherein A₁₀, A₂₀ and A₃₀ are as defined for formula IIIa₀ and X₁₀ is halogen. Suitable bromine and chlorine sources are bromine, chlorine, their succinimides such as N-bromosuccinimide (NBS), bromo- and chloro-acetamides and alkyl hypohalites. A preferred bromine source is bromine or NBS, and a preferred chlorine source is chlorine. In the case of bromination it is advantageous for the HBr that is formed to be removed from the reaction mixture, which may be accomplished, for example, by introducing an inert gas such as, for example, argon or nitrogen, beneath the surface of the reaction mixture. Incorporation of the halogens into the reaction mixture can be carried out by dropwise addition or direct introduction beneath the surface of the reaction mixture. In the case of direct introduction, the halogens can be diluted with an inert gas such as, for example, argon or nitrogen.

The reaction according to Reaction Step a) is preferably carried out in the presence of a free-radical initiator such as, for example, benzoyl peroxide or azoisobutyronitrile. Illumination of the reaction mixture is, moreover, advantageous. The halogenation is preferably carried out in the presence of azoisobutyronitrile.

The reaction is preferably carried out in the presence of a solvent. Suitable solvents are chlorobenzene, hexane, acetonitrile, tetrahydrofuran, methylcyclohexane or CCl₄ and also mixtures thereof; special preference is given to chlorobenzene or CCl₄.

The temperatures are generally from 0° C. to 150° C.; preference is given to a range from 80° C. to 130° C.

The reaction can also be carried out in a stepwise manner by introducing in a reaction step a,) one equivalent of the halogenating agent to produce a compound of the formula XX

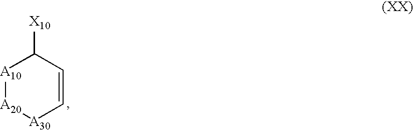

wherein A₁₀, A₂₀ and A₃₀ are as defined for formula IIIa₀ and X₁₀ is halogen, in reaction step a₂) addition of a second equivalent of halogenating agent to produce a compound of formula XXI

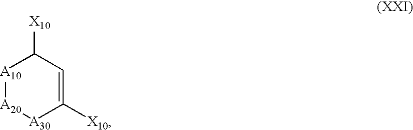

wherein A₁₀, A₂₀ and A₃₀ are as defined for formula IIIa₀ and X₁₀ is halogen, and in reaction step a₃) addition of a third equivalent of halogenating agent to produce a compound of formula XIX. In the reaction step b) the compound of formula XIX is converted to compound of formula IIIa₀ aqueous hydrolysis as described in WO 05105718 and WO 05/105717. Said sequence is summarised in scheme 9a.

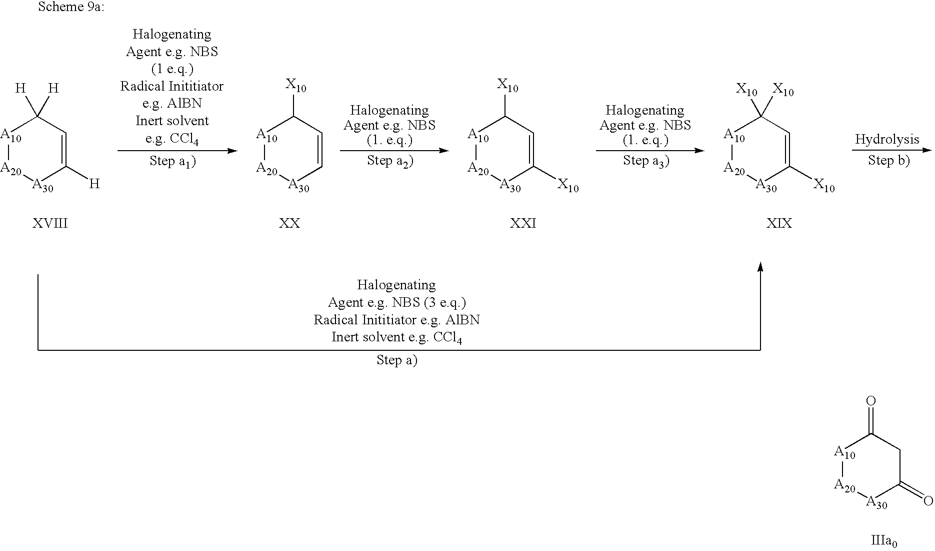

The formation of products XX, XXI and XIX can be monitored by proton NMR of the reaction mixture where the olefinc signals are particularly diagnostic (see preparative example P16).

Compounds of formula XXI,

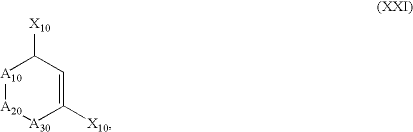

wherein A₁₀, A₂₀ and A₃₀ are as defined for formula IIIa₀ and X₁₀ is halogen, preferably bromo, are a further object of the present invention.

All other compounds of formula I, such as especially those of formula I-3, I-4, I-5, I-6, I-7 and I-8 can be prepared analogously to the processes described above.

The reactions to form compounds of formula I are advantageously carried out in aprotic, inert organic solvents. Such solvents are hydrocarbons, such as benzene, toluene, xylene or cyclohexane, chlorinated hydrocarbons, such as dichloromethane, trichloromethane, tetra-chloromethane or chlorobenzene, ethers, such as diethyl ether, ethylene glycol dimethyl ether, diethylene glycol dimethyl ether, tetrahydrofuran or dioxane, nitriles, such as aceto-nitrile or propionitrile, amides, such as N,N-dimethylformamide, diethylformamide or N-methylpyrrolidinone. The reaction temperatures are preferably from −20° C. to +120° C. If the reactions proceed slightly exothermically, they can generally be carried out at room temperature. In order to shorten the reaction time or to initiate the reaction, brief heating, up to the boiling point of the reaction mixture, can be carried out. The reaction times can likewise be shortened by the addition of suitable bases as reaction catalysts. As bases there are used especially the tertiary amines, such as trimethylamine, triethylamine, quinuclidine, 2-methyl-4-ethylpyridine, dimethylaminopyridine, 1,4-diazabicyclo[2.2.2]octane, 1,5-diazabicyclo-[4.3.0]non-5-ene or 1,5-diazabicyclo[5.4.0]undec-7-ene. It is also possible, however, to use as bases inorganic bases, such as hydrides, e.g. sodium or calcium hydride, hydroxides, e.g. dry sodium or potassium hydroxide, carbonates, e.g. sodium or potassium carbonate, or hydrogen carbonates, e.g. sodium or potassium hydrogen carbonate.

According to reaction schemes 6, 7 and 8, the compounds of formulae I and II are prepared using a chlorinating agent, e.g. thionyl chloride, phosgene, phosphorus pentachloride, phosphorus oxychloride or preferably oxalyl chloride. The reaction is preferably carried out in an inert organic solvent, for example in aliphatic, halogenated aliphatic, aromatic or halogenated aromatic hydrocarbons, for example n-hexane, benzene, toluene, xylenes, dichloromethane, 1,2-dichloroethane or chlorobenzene, at reaction temperatures in the range from −20° C. up to the reflux temperature of the reaction mixture, preferably at about from +40 to +100° C., and in the presence of a catalytic amount of N,N-dimethylformamide.

For the preparation of compounds of formulae I and IV according to Reaction Scheme 1 or with the aid of a coupling reagent, for example dicyclohexylcarbodiimide, (1-chloro-2-methyl-propenyl)-dimethylamine or 2-chloro-1-methylpyridinium iodide, according to Reaction Scheme 2, reaction is preferably likewise carried out in one of the inert organic solvents mentioned above at temperatures from about −20° C. to about +100° C., preferably from about +5° C. to about +50° C.

The end products of formula I can be isolated in conventional manner by concentration or evaporation of the solvent and purified by recrystallisation or trituration of the solid residue in solvents in which they are not readily soluble, such as ethers, aromatic hydrocarbons or chlorinated hydrocarbons, by distillation or by means of column chromatography or by means of the HPLC technique using a suitable eluant.

The sequence in which the reactions should be carried out in order as far as possible to avoid secondary reactions will also be familiar to the person skilled in the art. Unless the synthesis is specifically aimed at the isolation of pure isomers, the product may be obtained in the form of a mixture of two or more isomers, for example chiral centres in the case of alkyl groups or cis/trans isomerism in the case of alkenyl groups or <E> or <Z> forms, e.g. in respect of a —C(═NR₆)— group. All such isomers can be separated by methods known per se, for example chromatography, crystallisation, or produced in the desired form by means of a specific reaction procedure.

The compounds of formula I according to the invention can be used as herbicides in unmodified form, as obtained in the synthesis, but they are generally formulated into herbicidal compositions in a variety of ways using formulation adjuvants, such as carriers, solvents and surface-active substances. The formulations can be in various physical forms, for example in the form of dusting powders, gels, wettable powders, water-dispersible granules, water-dispersible tablets, effervescent compressed tablets, emulsifiable concentrates, microemulsifiable concentrates, oil-in-water emulsions, oil flowables, aqueous dispersions, oily dispersions, suspoemulsions, capsule suspensions, emulsifiable granules, soluble liquids, water-soluble concentrates (with water or a water-miscible organic solvent as carrier), impregnated polymer films or in other forms known, for example, from the Manual on Development and Use of FAO Specifications for Plant Protection Products, 5th Edition, 1999. Such formulations can either be used directly or are diluted prior to use. Diluted formulations can be prepared, for example, with water, liquid fertilisers, micronutrients, biological organisms, oil or solvents.

The formulations can be prepared, for example, by mixing the active ingredient with formulation adjuvants in order to obtain compositions in the form of finely divided solids, granules, solutions, dispersions or emulsions. The active ingredients can also be formulated with other adjuvants, for example finely divided solids, mineral oils, vegetable oils, modified vegetable oils, organic solvents, water, surface-active substances or combinations thereof. The active ingredients can also be contained in very fine microcapsules consisting of a polymer. Microcapsules contain the active ingredients in a porous carrier. This enables the active ingredients to be released into their surroundings in controlled amounts (e.g. slow release). Microcapsules usually have a diameter of from 0.1 to 500 microns. They contain active ingredients in an amount of about from 25 to 95% by weight of the capsule weight. The active ingredients can be present in the form of a monolithic solid, in the form of fine particles in solid or liquid dispersion or in the form of a suitable solution. The encapsulating membranes comprise, for example, natural and synthetic gums, cellulose, styrene-butadiene copolymers, polyacrylonitrile, polyacrylate, polyester, polyamides, polyureas, polyurethane or chemically modified polymers and starch xanthates or other polymers that are known to the person skilled in the art in this connection. Alternatively it is possible for very fine microcapsules to be formed wherein the active ingredient is present in the form of finely divided particles in a solid matrix of a base substance, but in that case the microcapsule is not encapsulated.

The formulation adjuvants suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylenes carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethyl hexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol (PEG 400), propionic acid, propyl lactate, propylene carbonate,propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and higher molecular weight alcohols, such as amyl alcohol, tetrahydrofurfuryl alcohol, hexanol, octanol, ethylene glycol, propylene glycol, glycerol, N-methyl-2-pyrrolidone and the like. Water is generally the carrier of choice for the dilution of the concentrates. Suitable solid carriers are, for example, talc, titanium dioxide, pyrophyllite clay, silica, attapulgite clay, kieselguhr, limestone, calcium carbonate, bentonite, calcium montomorillonite, cottonseed husks, wheatmeal, soybean flour, pumice, wood flour, ground walnut shells, lignin and similar materials, as described, for example, in CFR 180.1001. (c) & (d).

A large number of surface-active substances can advantageously be used both in solid and in liquid formulations, especially in those formulations which can be diluted with a carrier prior to use. Surface-active substances may be anionic, cationic, non-ionic or polymeric and they may be used as emulsifiying, wetting or suspending agents or for other purposes. Typical surface-active substances include, for example, salts of alkyl sulfates, such as diethanolammonium lauryl sulfate; salts of alkylarylsulfonates, such as calcium dodecyl-benzenesulfonate; alkylphenol-alkylene oxide addition products, such as nonylphenol ethoxylate; alcohol-alkylene oxide addition products, such as tridecyl alcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride, polyethylene glycol esters of fatty acids, such as polyethylene glycol stearate; block copolymers of ethylene oxide and propylene oxide; and salts of mono- and di-alkyl phosphate esters; and also further substances described e.g. in “McCutcheon's Detergents and Emulsifiers Annual”, MC Publishing Corp., Ridgewood, N.J., 1981.

Further adjuvants which can usually be used in pesticidal formulations include crystallisation inhibitors, viscosity-modifying substances, suspending agents, dyes, anti-oxidants, foaming agents, light absorbers, mixing aids, anti-foams, complexing agents, neutralising or pH-modifying substances and buffers, corrosion-inhibitors, fragrances, wetting agents, absorption improvers, micronutrients, plasticisers, glidants, lubricants, dispersants, thickeners, anti-freezes, microbiocides, and also liquid and solid fertilisers.

The formulations may also comprise additional active substances, for example further herbicides, herbicide safeners, plant growth regulators, fungicides or insecticides.

The compositions according to the invention can additionally include an additive comprising an oil of vegetable or animal origin, a mineral oil, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhône-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being important. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000.

The application and action of the oil additives can be further improved by combining them with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecyl-benzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltrisiloxanes, which are commercially available e.g. as Silwet L-77®, and also perfluorinated surfactants. The concentration of surface-active substances in relation to the total additive is generally from 1 to 30% by weight. Examples of oil additives that consist of mixtures of oils or mineral oils or derivatives thereof with surfactants are Edenor ME SU®, Turbocharge® (Syngenta AG, CH) and Actipron® (BP Oil UK Limited, GB).

The said surface-active substances may also be used in the formulations alone, that is to say without oil additives.

Furthermore, the addition of an organic solvent to the oil additive/surfactant mixture can contribute to a further enhancement of action. Suitable solvents are, for example, Solvesso® (ESSO) and Aromatic Solvent® (Exxon Corporation).The concentration of such solvents can be from 10 to 80% by weight of the total weight. Such oil additives, which may be in admixture with solvents, are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada.)

In addition to the oil additives listed above, in order to enhance the activity of the compositions according to the invention it is also possible for formulations of alkylpyrrolidones, (e.g. Agrimax®) to be added to the spray mixture. Formulations of synthetic latices, such as, for example, polyacrylamide, polyvinyl compounds or poly-1-p-menthene (e.g. Bond®, Courier® or Emerald®) can also be used. Solutions that contain propionic acid, for example Eurogkem Pen-e-trate®, can also be mixed into the spray mixture as activity-enhancing agents.

The herbicidal formulations generally contain from 0.1 to 99% by weight, especially from 0.1 to 95% by weight, of a compound of formula I and from 1 to 99.9% by weight of a formulation adjuvant, which preferably includes from 0 to 25% by weight of a surface-active substance. Whereas commercial products will preferably be formulated as concentrates, the end user will normally employ dilute formulations.

The rate of application of the compounds of formula I may vary within wide limits and depends upon the nature of the soil, the method of application (pre- or post-emergence; seed dressing; application to the seed furrow; no tillage application etc.), the crop plant, the weed or grass to be controlled, the prevailing climatic conditions, and other factors governed by the method of application, the time of application and the target crop. The compounds of formula I according to the invention are generally applied at a rate of 0.001 to 4 kg/ha, especially from 0.005 to 1 kg/ha.

Preferred formulations have especially the following compositions:

(%=percent by weight):

Emulsifiable Concentrates:

| | |
|---|---|
| active ingredient: | 1 to 95%, preferably 60 to 90% |
| surface-active agent: | 1 to 30%, preferably 5 to 20% |
| liquid carrier: | 1 to 80%, preferably 1 to 35% |

Dusts:

| | |
|---|---|
| active ingredient: | 0.1 to 10%, preferably 0.1 to 5% |
| solid carrier: | 99.9 to 90%, preferably 99.9 to 99% |

Suspension Concentrates:

| | |
|---|---|
| active ingredient: | 5 to 75%, preferably 10 to 50% |
| water: | 94 to 24%, preferably 88 to 30% |
| surface-active agent: | 1 to 40%, preferably 2 to 30% |

Wettable Powders:

| | |
|---|---|
| active ingredient: | 0.5 to 90%, preferably 1 to 80% |
| surface-active agent: | 0.5 to 20%, preferably 1 to 15% |
| solid carrier: | 5 to 95%, preferably 15 to 90% |

Granules:

| | |
|---|---|
| active ingredient: | 0.1 to 30%, preferably 0.1 to 15% |
| solid carrier: | 99.5 to 70%, preferably 97 to 85% |

The following Examples further illustrate, but do not limit, the invention.

| F1. Emulsifiable concentrates | a) | b) | c) | d) |
|---|---|---|---|---|
| active ingredient | 5% | 10% | 25% | 50% |
| calcium dodecylbenzene-sulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | — | 10% | 20% |
| arom. hydrocarbon | 85% | 78% | 55% | 16% |
| mixture C₉-C₁₂ | | | | |

Emulsions of any desired concentration can be prepared from such concentrates by dilution with water.

| F2. Solutions | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol | 20% | 10% | — | — |
| MW 400 | | | | |
| NMP | — | — | 30% | 10% |
| arom. hydrocarbon | 75% | 60% | — | — |
| mixture C₉-C₁₂ | | | | |

The solutions are suitable for application in the form of microdrops.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium | — | 6% | 5% | 6% |
| diisobutylnaphthalene- | | | | |
| sulfonate | | | | |
| octylphenol polyglycol | — | 1% | 2% | — |
| ether | | | | |
| (7-8 mol of ethylene oxide) | | | | |
| highly disperse silicic acid | 1% | 3% | 5% | 10% |
| kaolin | 88% | 62% | 35% | — |

The active ingredient is thoroughly mixed with the adjuvants and the mixture is thoroughly ground in a suitable mill, yielding wettable powders which can be diluted with water to give suspensions of any desired concentration.

| F4. Coated granules | a) | b) | c) |
|---|---|---|---|
| active ingredient | 0.1% | 5% | 15% |
| highly disperse silicic acid | 0.9% | 2% | 2% |
| inorg. carrier | 99.0% | 93% | 83% |
| (diameter 0.1-1 mm) | | | |
| e.g. CaCO₃ or SiO₂ | | | |

The active ingredient is dissolved in methylene chloride, the solution is sprayed onto the carrier and the solvent is subsequently evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly disperse silicic acid | 0.9% | 1% | 2% |
| inorg. carrier | 98.0% | 92% | 80% |
| (diameter 0.1-1 mm) | | | |
| e.g. CaCO₃ or SiO₂ | | | |

The finely ground active ingredient is applied uniformly, in a mixer, to the carrier moistened with polyethylene glycol. Non-dusty coated granules are obtained in this manner.

| F6. Extruder granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants and the mixture is moistened with water. The resulting mixture is extruded and then dried in a stream of air.

| F7. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F8. Suspension concentrates | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, yielding a suspension concentrate from which suspensions of any desired concentration can be prepared by dilution with water.

The invention relates also to a method for the selective control of grasses and weeds in crops of useful plants, which comprises treating the useful plants or the area under cultivation or the locus thereof with a compound of formula I.

Crops of useful plants in which the compounds according to the invention can be used include especially cereals, cotton, soybeans, sugar beet, sugar cane, plantation crops, rape, maize and rice.

The term “crops” is to be understood as including also crops that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popliae; or insecticidal proteins from Bacillus thuringiensis, such as δ-endotoxins, e.g. CryIA(b), CryIA(c), CryIF, CryIF(a2), CryIIA(b), CryIIA, CryIIIB(b1) or Cry9c, or vegetative insecticidal proteins (VIP), e.g. VIP1, VIP2, VIP3 or VIP3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsine inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example CryIA(b), CryIA(c), CryIF, CryIF(a2), CryIIA(b), CryIIIA, CryIIB(b1) or Cry9c, or vegetative insecticidal proteins (VIP), for example VIP1, VIP2, VIP3 or VIP3A, expressly also hybrid toxins, truncated toxins and modified toxins. Hybrid toxins are produced recombinantly by a new combination of different domains of those proteins (see, for example, WO 02/15701). Truncated toxins, for example a truncated CryIA(b), are known. In the case of modified toxins, one or more amino acids of the naturally occurring toxin are replaced. In such amino acid replacements, preferably non-naturally present protease recognition sequences are inserted into the toxin, such as, for example, in the case of CryIIIA055, a cathepsin-D-recognition sequence is inserted into a CryIIIA toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate mmonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); Nature-Gard® Agrisure® GT Advantage (GA21 glyphosate-tolerant trait), Agrisure® CB Advantage (Bt11 corn borer (CB) trait) and Protecta®.

Plant crops and their seed material can be resistant to herbicides and at the same time also to insect feeding (“stacked” transgenic events). Seed can, for example, have the ability to express an insecticidally active Cry3 protein and at the same time be glyphosate-tolerant. The term “crops” is to be understood as also including crops obtained as a result of conventional methods of breeding or genetic engineering which contain so-called output traits (e.g. improved flavour, storage stability, nutritional content).

Further Examples of such Transgenic Crops are:

1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated CryIA(b) toxin. Bt11 maize also transgenically expresses the enzyme PAT to achieve tolerance to the herbicide glufosinate ammonium.

2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a CryIA(b) toxin. Bt176 maize also transgenically expresses the enzyme PAT to achieve tolerance to the herbicide glufosinate ammonium.

3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified CryIIIA toxin. This toxin is Cry3A055 modified by insertion of a cathepsin-D-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a CryIIIB(b1) toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NU00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIA(b) toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Other indication areas for the active ingredients of the invention are the protection of stored products and stores and of material and, in the hygiene sector, especially the protection of domestic animals and livestock against pests of said type.

The weeds to be controlled may be both monocotyledonous and dicotyledonous weeds, such as, for example, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum halepense, Rottboellia, Cyperus, Abutilon, Sida, Xanthium, Amaranthus, Chenopodium, Ipomoea, Chrysanthemum, Galium, Viola and Veronica.

Areas under cultivation are to be understood as including land where the crop plants are already growing as well as land intended for the cultivation of those crop plants.

The following Examples illustrate the invention further but do not limit the invention.

### PREPARATION EXAMPLES

### Example P1

### Preparation of Ethoxycarbonimidoyl-acetic acid ethyl ester hydrochloride

20 ml (0.188 mol) of ethylcyanoacetate was taken up into 100 ml of diethylether, then 21 ml (0.376 mol) of ethanol was added. The reaction mixture was cooled in an ice/methanol bath to −5° C. and hydrogen chloride gas was bubbled through the reaction mixture until saturated. During tintroduction of the gas the temperature of the reaction mixture was kept below 15° C. The reaction mixture was allowed to warm to room temperature and left to stand over night. The white precipitate was collected by filtration and washed with ether and dried to give 23.3 g (63%) of pure ethoxycarbonimidoyl-acetic acid ethyl ester hydrochloride. 1H-NMR (CDCl3, ppm): 12.69 (s, 1H), 11.98 (1, 1H), 4.72 (q, J=7 Hz, 2H), 4.24 (q, J=7 Hz, 2H), 3.89 (s, 2H), 1.51 (t, J=7 Hz, 3H), 1.30 (t, J=7 Hz, 3H).

### Example P2

### Preparation of Ethoxycarbonimidoyl-acetic acid ethyl ester

25.7 g (0.131 mol) of ethoxycarbonimidoyl-acetic acid ethyl ester hydrochloride was suspended in 155 ml of dichlormethane and then a solution of 5.78 g (0.144 mol) of sodium hydroxide in 30 ml of water was added. After stirring for 30 min the layers were separated and the organics dried and vacced down to leave a colourless oil to give 19.35 g of clean Ethoxycarbonimidoyl-acetic acid ethyl ester. 1H-NMR (CDCl3, ppm): 6.28 (s, br, 1H), 4.19 (q, J=7 Hz, 2H), 4.02 (m, 2H), 3.37 (s, 1H), 3.25 (s, 1H), 1.30 (t, J=7 Hz, 3H), 1.26 (t, J=7 Hz, 3H).

### Example P3

### Preparation of 3-Amino-3-(formyl-hydrazono)-propionic acid ethyl ester

5.62 g (94.3 mmol) of formic hydrazide was suspended in 45 ml of ethanol, then 15 g (94.3 mmol) of ethoxycarbonimidoyl-acetic acid ethyl ester was added. The reaction mixture was briefly heated to −50° C., then stirred at room temperature for 6 hours during which time a white solid crashed out of solution. After standing over the weekend the solid was collected by filtration and washed with ether and dried. 1H-NMR (CDCl3, ppm): 10.50 (d, 1H), 8.45 (d, 1H), 5.45 (s, br, 2H), 3.23 (s, 2H), 4.20 (q, 2H), 1.28 (t, 3H).

### Example P4

### Preparation of 2-(N′-Formyl-hydrazino)-6-trifluoro methyl-nicotinic acid ethyl ester

7.97 g (40.7 mmol) of (E)-4-Butoxy-1,1,1-trifluoro-but-3-en-2-one and 7.043 g (40.7 mmol) of 3-Amino-3-(formyl-hydrazono)-propionic acid ethyl ester were taken up into 3 l of anhydrous ethanol and heated at reflux for 2 hours after which the reaction mixture was vacced down to leave a yellow oily solid. This solid was triturated with ether/hexane and the cream powder collected and dried, yielding pure product. 1H-NMR (CDCl3, ppm): 10.06 (d, 1H), 8.27 (s, 1H), 8.27 (s, 1H), 8.36 (d, 1H), 7.11 (d, 1H), 4.42 (q, 2H), 1.41 (t, 3H).

### Example P5

### Preparation of 5-Trifluoromethyl-[1,2,4]triazolo[4,3-a]pyridine-8-carboxylic acid ethyl ester

7.51 g (27.1 mmol) of 2-(N′-Formyl-hydrazino)-6-trifluoro methyl-nicotinic acid ethyl ester was suspended in toluene and 7.58 ml (81 mmol) phosphorous oxychloride added. The reaction mixture was heated at reflux for 5 hours, then allowed to cool to ambient temperature and left stand overnight. A gum had crashed out of solution. The suspension was added portion wise to a sodium bicarbonate solution with stirring. After complete addition of the reaction mixture, the solution was stirred for a further 20 mins before being extracted into ethyl acetate. The organics were dried and vacced down to leave a yellow solid. This solid was triturated using ether and a pale tan powder was collected by filtration, then further purified by bond-elute chromatography on a Flashmaster 2 (3:1→1:1 hexane:ethyl acetate→neat ethyl acetate) to give pure 5-Trifluoromethyl-[1,2,4]triazolo[4,3-a]pyridine-8-carboxylic acid ethyl ester. 1H-NMR (CDCl3, ppm): 9.05 (s, 1H), 8.10 (d, 1H), 7.42 (d, 1H), 4.60 (q, 2H), 1.50 (t, 3H).

### Example P6

### Preparation of potassium-(2Z,4E)-5-cyano-1,1,1-trifluoro-5-methoxycarbonyl-penta-2,4-dien-2-olate

45.2 g (163 mmol) of (E)-4-Butoxy-1,1,1-trifluoro-but-3-en-2-one and 14.25 ml (162 mmol) of methyl-cyanoacetate was taken up into toluene and 8.945 g (0.160 mmol) of solid potassium hydroxide added. After ˜25 mins a yellow solid had precipitated and the reaction had to be cooled to keep the temperature below 50° C. Upon cooling the reaction mixture was stirred at room temperature for 2 hours after which time the suspended yellow solid was collected by filtration, washed with toluene and dried to give potassium; (2Z,4E)-5-cyano-1,1,1-trifluoro-5-methoxycarbonyl-penta-2,4-dien-2-olate. 1H-NMR (DMSO, ppm): 8.16 (d, 1H), 5.54 (d, 1H), 3.61 (s, 3H).

### Example P7

### Preparation of 2-Bromo-6-trifluoromethyl-nicotinic acid methyl ester

Potassium-(2Z,4E)-5-cyano-1,1,1-trifluoro-5-methoxycarbonyl-penta-2,4-dien-2-olate (38.4 g (0.148 mol) was added portion wise to 130 ml of a 33% hydrogen bromide in acetic acid solution over 30 mins. The reaction mixture was stirred at room temperature for 3 hours before diluting with dichloromethane and water. The organics were vacced down to leave a yellow oil which was chromatagraphed using a vacuum column (hexane:ethyl acetate=5:1) to give pure product. 1H-NMR (CDCl3, ppm): 8.24 (d, 1H), 7.75 (d, 1H), 4.01 (s, 3H).

### Example P8

### Preparation of 2-Hydrazino-6-trifluoromethyl-nicotinic acid methyl ester

1 g (3.52 mmol) of 2-Bromo-6-trifluoromethyl-nicotinic acid methyl ester was taken up into 7 ml of dioxane and 0.43 ml (8.803 mmol) of hydrazine-monohydrate added. The reaction mixture was stirred at room temperature for 2.5 hours after which time the reaction mixture was diluted with water and extracted into ethyl acetate. The organics were dried and vacced down to leave an orange oil. Purification by Flashmaster chromatography using 20% ethyl acetate in hexane yields clean product. 1H-NMR (CDCl3, ppm): 8.87 (s, br, 1H), 8.25 (d, 1H), 6.93 (d, 1H), 4.09 (s, br, 2H), 3.91 (s, 3H).

### Example P9

### Preparation of 2-(N′-Propionyl-hydrazino)-6-trifluoromethyl-nicotinic acid methyl ester

1.586 g (6.75 mmol) 2-Hydrazino-6-trifluoromethyl-nicotinic acid methyl ester was taken up into 15 ml THF and 0.94 ml (6.75 mmol) triethylamine was added. The solution was cooled to ˜0° C. in an ice/ethanol bath. Propionyl chloride (0.6 ml, 6.75 mmol) was taken up into 5 ml THF and added dropwise over 10 mins. The reaction mixture was allowed to warm to room temperature then stirred for a further 2 hours, after which the reaction mixture was diluted with water, then extracted into ethyl acetate. The organics were vacced down to leave a yellow solid which was clean product. 1H-NMR (CDCl3, ppm): 9.79 (s, br, 1H), 8.32 (d, 1H), 7.92 (s, br, 1H), 7.07 (d, 1H), 3.95 (s, 3H), 2.36 (m, 2H), 1.24 (t, 2H).

### Example P10

### Preparation of 3-Isopropyl-5-trifluoromethyl-[1,2,4]triazolo[4,3-a]pyridine-8-carboxylic acid

1.804 g (6.28 mmol) 3-Isopropyl-5-trifluoromethyl-[1,2,4]triazolo[4,3-a]pyridine-8-carboxylic acid methyl ester was suspended in 11 ml of dioxane at room temperature. 0.423 g (7.54 mmol) potassium hydroxide was dissolved in 9 ml of water and added to the reaction mixture, resulting in an immediate red colouration as all the solids went into solution. The reaction mixture was stirred for 1.5 hours, after which the reaction mixture was taken to pH ˜1.5 by addition of dilute HCl. The reaction mixture was extracted into ethyl acetate and the organics dried and vacced down to leave a yellow solid. Trituration with ether yielded an off-white solid which was clean product. 1H-NMR (CDCl3, ppm): 8.23 (d, 1H), 7.60 (d, 1H), 3.68 (m, 1H), 1.55 (d, 6H)

### Example P11

### Preparation of 3-(3-Methyl-5-trifluoromethyl-[1,2,4]triazolo[4,3-a]pyridine-8-carbonyl)-bicyclo[3.2.1]octane-2,4-dione

1 g (4.08 mmol) of 3-methyl-5-trifluoromethyl-[1,2,4]triazolo[4,3-a]pyridine-8-carboxylic acid was suspended in 4 ml of acetonitrile, then 0.567 g (4.082 mmol) p-nitrophenol was added followed by 0.925 g (4.49 mmol) of DCC. After a few minutes the reaction mixture formed a thick paste, so a further 2 ml of acetonitrile was added. After 3 hours of stirring at room temperature 0.563 g (4.082 mmol) bicyclo[3.2.1]octane-2,4-dione was added, followed by 1.42 ml (10.2 mmol) of triethylamine and 40 ul (0.4082 mmol) of acetone-cyanohydrine. The reaction mixture was further stirred at room temperature for 4 hours then left to stand over night. The crude reaction mixture was then put onto a pre-wet bond elute cartridge which was eluted with a 500 ml:90 ml:30 ml mixture of ethyl acetate:methanol:acetic acid. The desired product fractions were vacced down to leave an orange oil. Trituration with acetone/hexane yielded the pure product as a pale pink powder. LCMS (Waters, ZQ): Rt=1.19 min, M+H=366); 1H-NMR (CDCl₃, ppm): 7.41 (d, 1H), 7.21 (d, 1H), 2.98 (m, 2H), 2.88 (m, 3H), 1.7-2.4 (m, 6H).

### Example P12

### Preparation of 3-Hydroxy-5-trifluoromethyl-[1,2,4]triazolo[4,3-a]pyridine-8-carboxylic acid methyl ester

3 ml of a 20% phosgene in toluene solution was added to 140 mg (0.596 mmol) 2-hydrazino-6-trifluoromethyl-nicotinic acid methyl ester and the reaction mixture was heated for 1 hour at reflux after which time the initial solid had gone into solution and a yellow solid had crashed out of solution. Upon cooling, this solid was collected by filtration and washed with ether and dried in vacuo yielding pure product. 1H-NMR (DMSO, ppm): 10.26 (s, br, 1H), 7.87 (d, 1H), 7.01 (d, 1H), 4.03 (s, 3H).

### Example P13

### Preparation of 5-Difluoromethyl-3-methyl-[1,2,4]triazolo[4,3-a]pyridine-8-carboxylic acid ethyl ester

0.5 g of 5% Pd on carbon was weighed into the hydrogenation vessel and 2.5 g (8.63 mmol) 5-(Chloro-difluoro-methyl)-3-methyl-[1,2,4]triazolo[4,3-a]pyridine-8-carboxylic acid ethyl ester added. 15 ml of ethanol was carefully added, followed by 1.2 ml (8.63 mmol) of triethylamine. The reaction mixture was subjected to hydrogenation at 1 Bar pressure for 2 hours. The reaction mixture was diluted with water, the catalyst removed by filtration and the residue washed through copiously with ethyl acetate. The organics were separated, dried and vacced down to leave pale brown solid. The solid was triturated using ether and the product was isolated as a pale orange powder. 1H-NMR (CDCl3, ppm): 10.7.96 (d, 1H), 7.22 (d, 1H), 7.11 (t, 1H), 4.56 (t, 2H), 2.99 (s, 3H), 1.47 (q, 3H).

### Example P14

### Preparation of 2-Methyl-5-trifluoromethyl-[1,2,4]triazolo[1,5-a]pyridine-8-carboxylic acid

0.2 g (0.706 mmol) potassium 3-Methyl-5-trifluoromethyl-[1,2,4]triazolo[4,3-a]pyridine-8-carboxylate was suspended in 2.5M sodium hydroxide solution and heated in the microwave at 100° C. for 5 mins upon which all solids went into solution. The solution was diluted to ˜pH 2 by the addition of dilute hydrochloric acid. After a few minutes a white solid crashed out of solution. This solid was collected by filtration and dried on the sinter. 1H-NMR (CDCl3, ppm): 8.45 (d, 1H), 7.62 (d, 1H), 2.74 (s, 3H).

### Example P15

### Preparation of 6-Bromo-5-trifluoromethyl-pyridine-2-carboxylic acid ethyl ester

9.41 g (40 mMol) 6-hydroxy-5-trifluoromethyl-pyridine-2-carboxylic acid ethyl ester was heated together with 14.91 g (52 mMol) phosphorousoxybromide in the presence of an 0.15 ml of dimethylformamide to 110° C. After 90 minutes the crude material could dawn to 40° C. was transferred under intensive stirring into cold water at below 25° C. The product was isolated by extraction with ethylacetate and dried. After chromatographic purification on silicagel (eluent:ethylacetate/hexane 1:3) 6-bromo-5-trifluoromethyl-pyridine-2-carboxylic acid ethyl ester was obtained as crystalline product of m.p. 40-40.5° C.; ¹H-NMR (CDCl₃, ppm): 8.18 (d, 1H), 8.12 (d, 1H), 4.51 (q, 2H), 1.44 (t, 3H).

### Example P16

### 2,4,4-tribromo-bicyclo[3.2.1]oct-2-ene

To a solution of 1.08 g (10 mmol) of bicyclo[3.2.1]oct-2-ene in 50 ml of CCl₄ there are added, under a nitrogen atmosphere, 0.16 g (1 mmol) of azoisobutyronitrile. The reaction mixture is then illuminated with a strong lamp and heated to reflux, with stirring. To this mixture is added 1.96 g (11 mmol) of N-bromosuccinimide (NBS) and stirring is carried out for 4 hours at reflux. After this time sample of the reaction mixture can be analysed directly by ¹H NMR in chloroform whereby 4-Bromo-bicyclo[3.2.1]oct-2-ene is seen to be the major product (Selected ¹H-NMR (CDCl₃, ppm): 5.98 (dd, 1H), 5.61 (m, 1H), 4.60 (m, 1H)).

A further 1.96 g (11 mmol) of N-bromosuccinimide (NBS) are added and the reaction mixture is maintained at reflux, with stirring for a further 2 hours. Analysis of the reaction mixture by ¹H-NMR after this time shows 2,4-Dibromo-bicyclo[3.2.1]oct-2-ene to be the major product (Selected signals ¹H-NMR (CDCl₃, ppm): 5.95 (d, 1H), 4.55 (m, 1H)). A further 1.96 g (11 mmol) of N-bromosuccinimide (NBS) are added and the reaction mixture is maintained at reflux, until analysis of the reaction mixture by ¹H-NMR shows reaction completion. The reaction mixture is then cooled to ambient temperature and diluted with hexane. After filtration and removal of the solvent in vacuo, 3.7 g crude of 2,4,4-tribromo-bicyclo[3.2.1]oct-2-ene are obtained as a red-brown oil.

¹H NMR (CDCl₃): 6.35 (s, 1H), 3.20 (d, 1H), 2.70-2.80 (d, 1H), 2.55-2.60 (d, 1H), 1.85-2.20 (m, 4H), 1.55-1.65 (m, 1H).

The following Tables list preferred compounds of formula 1. The following definitions apply: “Me” represents the methyl group, “Ph” the phenyl group, BIOD, CHD, 5Me-CHD, Sync, NMe-py and IFT as defined in scheme 10.

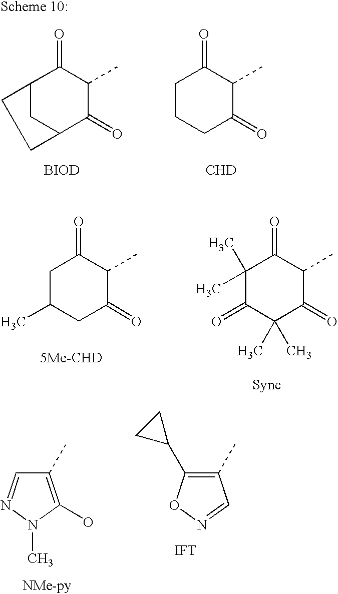

LCMS-data for physico-chemical characterization were obtained on an analytical Waters LC-MS instrument (W2790, ZMD-2000). Column was an Atlantis dC18, 3 um 3.0 mm×20 mm. Solvents were: A=0.1% formic acid in water, B=0.1% formic acid in acetonitrile. Gradient was 10% to 90% B in 2.5 min; flow rate was 2 ml/min. Physicochemical data are reported in the following format: retention time (min); M found in positive ionisation mode (m/z⁺, M found in negative ionisation mode (m/z⁻).

The compounds of the following tables can be prepared analogously:

TABLE 1
Compounds of formula I-1a:

| | |
|---|---|
| | (I-1a) |
| 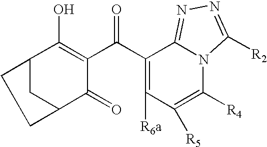 | |

| Cmpd No. | R₂ | R₄ | R₅ | R₆a | Phys/chem data |
|---|---|---|---|---|---|
| 1.1 | H | CF₃ | H | H | 1.03 min; 352, |
| | | | | | 350 |
| 1.2 | Cl | CF₃ | H | H | 1.32; 388, 386 |
| 1.3 | Br | CF₃ | H | H | |
| 1.4 | CH₃ | CF₃ | H | H | see example |
| | | | | | [P11] |
| 1.5 | CH₂CH₃ | CF₃ | H | H | 1.29 min; 380, |
| | | | | | 378 |
| 1.6 | (CH₂)₅CH₃ | CF₃ | H | H | |
| 1.7 | CH(CH₃)₂ | CF₃ | H | H | 1.34 min; 394 |
| 1.8 | cyclopropyl | CF₃ | H | H | 1.34 min; 392, |
| | | | | | 390 |
| 1.9 | CCH | CF₃ | H | H | |
| 1.10 | CH═CH₂ | CF₃ | H | H | |
| 1.11 | CH₂CH═CH₂ | CF₃ | H | H | |
| 1.12 | CH₂F | CF₃ | H | H | |
| 1.13 | CHFCH₃ | CF₃ | H | H | |
| 1.14 | CHF₂ | CF₃ | H | H | |
| 1.15 | CH₂OH | CF₃ | H | H | |
| 1.16 | CH₂OCH₃ | CF₃ | H | H | 1.16 min; 396, |
| | | | | | 394 |
| 1.17 | CH₂CH₂OCH₃ | CF₃ | H | H | |
| 1.18 | CH₂OCOCH₃ | CF₃ | H | H | |
| 1.19 | CH₂OCOPh | CF₃ | H | H | |
| 1.20 | CO₂Me | CF₃ | H | H | |
| 1.21 | CH₂SMe | CF₃ | H | H | |
| 1.22 | CH₂SO₂Me | CF₃ | H | H | |
| 1.23 | CH₂SO₂Ph | CF₃ | H | H | |
| 1.24 | CH₂SOMe | CF₃ | H | H | |
| 1.25 | CH₂—O—CH₂-(2-tetrahydrofuryl) | CF₃ | H | H | |
| 1.26 | NH2 | CF₃ | H | H | |
| 1.27 | N(CH₃)₂ | CF₃ | H | H | |
| 1.28 | NHC(O)Me | CF₃ | H | H | |
| 1.29 | OCH₃ | CF₃ | H | H | |
| 1.30 | OCH₂CCH | CF₃ | H | H | |
| 1.31 | O(CH2)2OMe | CF₃ | H | H | |
| 1.32 | OCHF₂ | CF₃ | H | H | |
| 1.33 | OPh | CF₃ | H | H | |
| 1.34 | Ph | CF₃ | H | H | |
| 1.35 | 4-Cl-Ph | CF₃ | H | H | 1.56 min; 462, |
| | | | | | 460 |
| 1.36 | SH | CF₃ | H | H | |
| 1.37 | SCH₂Ph | CF₃ | H | H | |
| 1.38 | SCH₃ | CF₃ | H | H | |
| 1.39 | SO₂CH₂Ph | CF₃ | H | H | |
| 1.40 | SO₂CH₃ | CF₃ | H | H | |
| 1.41 | SO₂N(CH₃)₂ | CF₃ | H | H | |
| 1.42 | SO₂Ph | CF₃ | H | H | |
| 1.43 | SOCH₂Ph | CF₃ | H | H | |
| 1.44 | SOCH₃ | CF₃ | H | H | |
| 1.45 | SOPh | CF₃ | H | H | |
| 1.46 | SPh | CF₃ | H | H | |
| 1.47 | 2-furyl | CF₃ | H | H | |
| 1.48 | 2-pyridyl | CF₃ | H | H | |
| 1.49 | 3-pyridyl | CF₃ | H | H | |
| 1.50 | 4-pyridyl | CF₃ | H | H | |
| 1.51 | H | CF₂H | H | H | |
| 1.52 | Cl | CF₂H | H | H | |
| 1.53 | Br | CF₂H | H | H | |
| 1.54 | CH₃ | CF₂H | H | H | 1.05 min; 348, |
| | | | | | 346 |
| 1.55 | CH₂CH₃ | CF₂H | H | H | |
| 1.56 | (CH₂)₅CH₃ | CF₂H | H | H | |
| 1.57 | CH(CH₃)₂ | CF₂H | H | H | |
| 1.58 | cyclopropyl | CF₂H | H | H | |
| 1.59 | CCH | CF₂H | H | H | |
| 1.60 | CH═CH₂ | CF₂H | H | H | |
| 1.61 | CH₂CH═CH₂ | CF₂H | H | H | |
| 1.62 | CH₂F | CF₂H | H | H | |
| 1.63 | CHFCH₃ | CF₂H | H | H | |
| 1.64 | CHF₂ | CF₂H | H | H | |
| 1.65 | CH₂OH | CF₂H | H | H | |
| 1.66 | CH₂OCH₃ | CF₂H | H | H | |
| 1.67 | CH₂CH₂OCH₃ | CF₂H | H | H | |
| 1.68 | CH₂OCOCH₃ | CF₂H | H | H | |
| 1.69 | CH₂OCOPh | CF₂H | H | H | |
| 1.70 | CO₂Me | CF₂H | H | H | |
| 1.71 | CH₂SMe | CF₂H | H | H | |
| 1.72 | CH₂SO₂Me | CF₂H | H | H | |
| 1.73 | CH₂SO₂Ph | CF₂H | H | H | |
| 1.74 | CH₂SOMe | CF₂H | H | H | |
| 1.75 | CH₂—O—CH₂-(2-tetrahydrofuryl) | CF₂H | H | H | |
| 1.76 | NH₂ | CF₂H | H | H | |
| 1.77 | N(CH₃)₂ | CF₂H | H | H | |
| 1.78 | NHC(O)Me | CF₂H | H | H | |
| 1.79 | OCH₃ | CF₂H | H | H | |
| 1.80 | OCH₂CCH | CF₂H | H | H | |
| 1.81 | O(CH₂)₂OMe | CF₂H | H | H | |
| 1.82 | OCHF₂ | CF₂H | H | H | |
| 1.83 | OPh | CF₂H | H | H | |
| 1.84 | Ph | CF₂H | H | H | |
| 1.85 | 4-Cl-Ph | CF₂H | H | H | |
| 1.86 | SH | CF₂H | H | H | |
| 1.87 | SCH₂Ph | CF₂H | H | H | |
| 1.88 | SCH₃ | CF₂H | H | H | |
| 1.89 | SO₂CH₂Ph | CF₂H | H | H | |
| 1.90 | SO₂CH₃ | CF₂H | H | H | |
| 1.91 | SO₂N(CH₃)₂ | CF₂H | H | H | |
| 1.92 | SO₂Ph | CF₂H | H | H | |
| 1.93 | SOCH₂Ph | CF₂H | H | H | |
| 1.94 | SOCH₃ | CF₂H | H | H | |
| 1.95 | SOPh | CF₂H | H | H | |
| 1.96 | SPh | CF₂H | H | H | |
| 1.97 | 2-furyl | CF₂H | H | H | |
| 1.98 | 2-pyridyl | CF₂H | H | H | |
| 1.99 | 3-pyridyl | CF₂H | H | H | |
| 1.100 | 4-pyridyl | CF₂H | H | H | |
| 1.101 | H | Me | H | H | |
| 1.102 | Br | Me | H | H | |
| 1.103 | CH₃ | Me | H | H | |
| 1.104 | (CH₂)₅CH₃ | Me | H | H | |
| 1.105 | CH(CH₃)₂ | Me | H | H | |
| 1.106 | cyclopropyl | Me | H | H | |
| 1.107 | CCH | Me | H | H | |
| 1.108 | CH₂CH═CH₂ | Me | H | H | |
| 1.109 | CHFCH₃ | Me | H | H | |
| 1.110 | CH₂OH | Me | H | H | |
| 1.111 | CH₂OCH₃ | Me | H | H | |
| 1.112 | CH₂OCOCH₃ | Me | H | H | |
| 1.113 | CO₂Me | Me | H | H | |
| 1.114 | CH₂SMe | Me | H | H | |
| 1.115 | CH₂SO₂Me | Me | H | H | |
| 1.116 | CH₂—O—CH₂-(2-tetrahydrofuryl) | Me | H | H | |
| 1.117 | NH₂ | Me | H | H | |
| 1.118 | N(CH₃)₂ | Me | H | H | |
| 1.119 | NHC(O)Me | Me | H | H | |
| 1.120 | OCH₃ | Me | H | H | |
| 1.121 | O(CH₂)₂OMe | Me | H | H | |
| 1.122 | OCHF₂ | Me | H | H | |
| 1.123 | OPh | Me | H | H | |
| 1.124 | 4-Cl-Ph | Me | H | H | |
| 1.125 | SH | Me | H | H | |
| 1.126 | SCH₃ | Me | H | H | |
| 1.127 | SO₂CH₃ | Me | H | H | |
| 1.128 | SO₂N(CH₃)₂ | Me | H | H | |
| 1.129 | SO₂Ph | Me | H | H | |
| 1.130 | 3-pyridyl | Me | H | H | |
| 1.131 | Br | CHFCH₃ | H | H | |
| 1.132 | CH₃ | CHFCH₃ | H | H | |
| 1.133 | CH(CH₃)₂ | CHFCH₃ | H | H | |
| 1.134 | cyclopropyl | CHFCH₃ | H | H | |
| 1.135 | CCH | CHFCH₃ | H | H | |
| 1.136 | CH₂CH═CH₂ | CHFCH₃ | H | H | |
| 1.137 | CHFCH₃ | CHFCH₃ | H | H | |
| 1.138 | CH₂OCH₃ | CHFCH₃ | H | H | |
| 1.139 | CH₂OCOCH₃ | CHFCH₃ | H | H | |
| 1.140 | CH₂SMe | CHFCH₃ | H | H | |
| 1.141 | NH₂ | CHFCH₃ | H | H | |
| 1.142 | N(CH₃)₂ | CHFCH₃ | H | H | |
| 1.143 | NHC(O)Me | CHFCH₃ | H | H | |
| 1.144 | OCH₃ | CHFCH₃ | H | H | |
| 1.145 | O(CH₂)₂OMe | CHFCH₃ | H | H | |
| 1.146 | OCHF₂ | CHFCH₃ | H | H | |
| 1.147 | 4-Cl-Ph | CHFCH₃ | H | H | |
| 1.148 | SCH₃ | CHFCH₃ | H | H | |
| 1.149 | SO₂CH₃ | CHFCH₃ | H | H | |
| 1.150 | 3-pyridyl | CHFCH₃ | H | H | |
| 1.151 | Br | CF(CH₃)₂ | H | H | |
| 1.152 | CH₃ | CF(CH₃)₂ | H | H | |
| 1.153 | CH(CH₃)₂ | CF(CH₃)₂ | H | H | |
| 1.154 | cyclopropyl | CF(CH₃)₂ | H | H | |
| 1.155 | CCH | CF(CH₃)₂ | H | H | |
| 1.156 | CH₂CH═CH₂ | CF(CH₃)₂ | H | H | |
| 1.157 | CHFCH₃ | CF(CH₃)₂ | H | H | |
| 1.158 | CH₂OCH₃ | CF(CH₃)₂ | H | H | |
| 1.159 | CH₂SMe | CF(CH₃)₂ | H | H | |
| 1.160 | N(CH₃)₂ | CF(CH₃)₂ | H | H | |
| 1.161 | NHC(O)Me | CF(CH₃)₂ | H | H | |
| 1.162 | OCH₃ | CF(CH₃)₂ | H | H | |
| 1.163 | O(CH₂)₂OMe | CF(CH₃)₂ | H | H | |
| 1.164 | OCHF₂ | CF(CH₃)₂ | H | H | |
| 1.165 | 4-Cl-Ph | CF(CH₃)₂ | H | H | |
| 1.166 | SCH₃ | CF(CH₃)₂ | H | H | |
| 1.167 | 3-pyridyl | CF(CH₃)₂ | H | H | |
| 1.168 | Br | CN | H | H | |
| 1.169 | CH₃ | CN | H | H | |
| 1.170 | CH₂OCH₃ | CN | H | H | |
| 1.171 | CH₂SMe | CN | H | H | |
| 1.172 | N(CH₃)₂ | CN | H | H | |
| 1.173 | OCH₃ | CN | H | H | |
| 1.174 | OCHF₂ | CN | H | H | |
| 1.175 | 4-Cl-Ph | CN | H | H | |
| 1.176 | SCH₃ | CN | H | H | |
| 1.177 | 3-pyridyl | CN | H | H | |
| 1.178 | Br | triazolyl | H | H | |
| 1.179 | CH₃ | triazolyl | H | H | |
| 1.180 | Br | CF₃ | Me | H | |
| 1.181 | CH₃ | CF₃ | Me | H | |
| 1.182 | CH(CH₃)₂ | CF₃ | Me | H | |
| 1.183 | cyclopropyl | CF₃ | Me | H | |
| 1.184 | CCH | CF₃ | Me | H | |
| 1.185 | CH₂CH═CH₂ | CF₃ | Me | H | |
| 1.186 | CHFCH₃ | CF₃ | Me | H | |
| 1.187 | CH₂OCH₃ | CF₃ | Me | H | |
| 1.188 | CH₂SMe | CF₃ | Me | H | |
| 1.189 | N(CH₃)₂ | CF₃ | Me | H | |
| 1.190 | NHC(O)Me | CF₃ | Me | H | |
| 1.191 | OCH₃ | CF₃ | Me | H | |
| 1.192 | O(CH2)2OMe | CF₃ | Me | H | |
| 1.193 | OCHF₂ | CF₃ | Me | H | |
| 1.194 | 4-Cl-Ph | CF₃ | Me | H | |
| 1.195 | SCH₃ | CF₃ | Me | H | |
| 1.196 | 3-pyridyl | CF₃ | Me | H | |
| 1.197 | Br | CF₃ | Cl | H | |
| 1.198 | CH₃ | CF₃ | Cl | H | |
| 1.199 | CH₂OCH₃ | CF₃ | Cl | H | |
| 1.200 | CH₂SMe | CF₃ | Cl | H | |
| 1.201 | N(CH₃)₂ | CF₃ | Cl | H | |
| 1.202 | OCH₃ | CF₃ | Cl | H | |
| 1.203 | OCHF₂ | CF₃ | Cl | H | |
| 1.204 | 4-Cl-Ph | CF₃ | Cl | H | |
| 1.205 | SCH₃ | CF₃ | Cl | H | |
| 1.206 | 3-pyridyl | CF₃ | Cl | H | |
| 1.207 | Br | CF₃ | OMe | H | |
| 1.208 | CH₃ | CF₃ | OMe | H | |
| 1.209 | t-butyl | CF₃ | H | H | 1.54 min; 408 |
| 1.210 | Me | H | H | H | 1.36 mins; 298, |
| | | | | | 296 |
| 1.211 | OEt | CF₃ | H | H | 1.42 min, 396, |
| | | | | | 394 |
| 1.212 | SEt | CF₃ | H | H | 1.50 min; 412, |
| | | | | | 410 |
| 1.213 | Cl | CF₃ | H | CN | 1.16 min; 413, |
| | | | | | 411 |

TABLE 2
Compounds of formula I-2a:

| | |
|---|---|
| | (I-2a) |
| 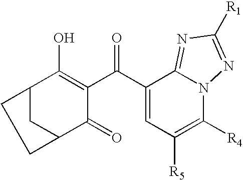 | |

| Cmpd No | R₁ | R₄ | R₅ | Phys./chem data |
|---|---|---|---|---|
| 2.1 | H | CF₃ | H | 1.22 min; 352, 350 |
| 2.2 | Cl | CF₃ | H | 1.54 min; 388, 386 |
| 2.3 | Br | CF₃ | H | |
| 2.4 | CH₃ | CF₃ | H | 1.32 min; 366, 364 |
| 2.5 | CH₂CH₃ | CF₃ | H | |
| 2.6 | (CH₂)₅CH₃ | CF₃ | H | |
| 2.7 | CH(CH₃)₂ | CF₃ | H | 1.64 min; 394, 392 |
| 2.8 | cyclopropyl | CF₃ | H | |
| 2.9 | C≡CH | CF₃ | H | |
| 2.10 | CH═CH₂ | CF₃ | H | |
| 2.11 | CH₂CH═CH₂ | CF₃ | H | |
| 2.12 | CH₂F | CF₃ | H | |
| 2.13 | CHFCH₃ | CF₃ | H | |
| 2.14 | CHF₂ | CF₃ | H | |
| 2.15 | CH₂OH | CF₃ | H | |
| 2.16 | CH₂OCH₃ | CF₃ | H | |
| 2.17 | CH₂CH₂OCH₃ | CF₃ | H | |
| 2.18 | CH₂OCOCH₃ | CF₃ | H | |
| 2.19 | CH₂OCOPh | CF₃ | H | |
| 2.20 | CO₂Me | CF₃ | H | |
| 2.21 | CH₂SMe | CF₃ | H | |
| 2.22 | CH₂SO₂Me | CF₃ | H | |
| 2.23 | CH₂SO₂Ph | CF₃ | H | |
| 2.24 | CH₂SOMe | CF₃ | H | |
| 2.25 | CH₂—O—CH₂-(2-tetrahydrofuryl) | CF₃ | H | |
| 2.26 | NH₂ | CF₃ | H | |
| 2.27 | N(CH₃)₂ | CF₃ | H | |
| 2.28 | NHC(O)Me | CF₃ | H | |
| 2.29 | OCH₃ | CF₃ | H | |
| 2.30 | OCH₂C≡CH | CF₃ | H | |
| 2.31 | O(CH₂)₂OMe | CF₃ | H | |
| 2.32 | OCHF₂ | CF₃ | H | |
| 2.33 | OPh | CF₃ | H | |
| 2.34 | Ph | CF₃ | H | |
| 2.35 | 4-Cl-Ph | CF₃ | H | 1.89 min; 462 |
| 2.36 | SH | CF₃ | H | |
| 2.37 | SCH₂Ph | CF₃ | H | |
| 2.38 | SCH₃ | CF₃ | H | |
| 2.39 | SO₂CH₂Ph | CF₃ | H | |
| 2.40 | SO₂CH₃ | CF₃ | H | |
| 2.41 | SO₂N(CH₃)₂ | CF₃ | H | |
| 2.42 | SO₂Ph | CF₃ | H | |
| 2.43 | SOCH₂Ph | CF₃ | H | |
| 2.44 | SOCH₃ | CF₃ | H | |
| 2.45 | SOPh | CF₃ | H | |
| 2.46 | SPh | CF₃ | H | |
| 2.47 | 2-furyl | CF₃ | H | |
| 2.48 | 2-pyridyl | CF₃ | H | |
| 2.49 | 3-pyridyl | CF₃ | H | |
| 2.50 | 4-pyridyl | CF₃ | H | |
| 2.51 | H | CF₂H | H | |
| 2.52 | Cl | CF₂H | H | |
| 2.53 | Br | CF₂H | H | |
| 2.54 | CH₃ | CF₂H | H | |
| 2.55 | CH₂CH₃ | CF₂H | H | |
| 2.56 | (CH₂)₅CH₃ | CF₂H | H | |
| 2.57 | CH(CH₃)₂ | CF₂H | H | |
| 2.58 | cyclopropyl | CF₂H | H | |
| 2.59 | C≡CH | CF₂H | H | |
| 2.60 | CH═CH₂ | CF₂H | H | |
| 2.61 | CH₂CH═CH₂ | CF₂H | H | |
| 2.62 | CH₂F | CF₂H | H | |
| 2.63 | CHFCH₃ | CF₂H | H | |
| 2.64 | CHF₂ | CF₂H | H | |
| 2.65 | CH₂OH | CF₂H | H | |
| 2.66 | CH₂OCH₃ | CF₂H | H | |
| 2.67 | CH₂CH₂OCH₃ | CF₂H | H | |
| 2.68 | CH₂OCOCH₃ | CF₂H | H | |
| 2.69 | CH₂OCOPh | CF₂H | H | |
| 2.70 | CO₂Me | CF₂H | H | |
| 2.71 | CH₂SMe | CF₂H | H | |
| 2.72 | CH₂SO₂Me | CF₂H | H | |
| 2.73 | CH₂SO₂Ph | CF₂H | H | |
| 2.74 | CH₂SOMe | CF₂H | H | |
| 2.75 | CH₂—O—CH₂-(2-tetrahydrofuryl) | CF₂H | H | |
| 2.76 | NH₂ | CF₂H | H | |
| 2.77 | N(CH₃)₂ | CF₂H | H | |
| 2.78 | NHC(O)Me | CF₂H | H | |
| 2.79 | OCH₃ | CF₂H | H | |
| 2.80 | OCH₂C≡CH | CF₂H | H | |
| 2.81 | O(CH₂)₂OMe | CF₂H | H | |
| 2.82 | OCHF₂ | CF₂H | H | |
| 2.83 | OPh | CF₂H | H | |
| 2.84 | Ph | CF₂H | H | |
| 2.85 | 4-Cl-Ph | CF₂H | H | |
| 2.86 | SH | CF₂H | H | |
| 2.87 | SCH₂Ph | CF₂H | H | |
| 2.88 | SCH₃ | CF₂H | H | |
| 2.89 | SO₂CH₂Ph | CF₂H | H | |
| 2.90 | SO₂CH₃ | CF₂H | H | |
| 2.91 | SO₂N(CH₃)₂ | CF₂H | H | |
| 2.92 | SO₂Ph | CF₂H | H | |
| 2.93 | SOCH₂Ph | CF₂H | H | |
| 2.94 | SOCH₃ | CF₂H | H | |
| 2.95 | SOPh | CF₂H | H | |
| 2.96 | SPh | CF₂H | H | |
| 2.97 | 2-furyl | CF₂H | H | |
| 2.98 | 2-pyridyl | CF₂H | H | |
| 2.99 | 3-pyridyl | CF₂H | H | |
| 2.100 | 4-pyridyl | CF₂H | H | |
| 2.101 | H | Me | H | |
| 2.102 | Br | Me | H | |
| 2.103 | CH₃ | Me | H | |
| 2.104 | (CH₂)₅CH₃ | Me | H | |
| 2.105 | CH(CH₃)₂ | Me | H | |
| 2.106 | cyclopropyl | Me | H | |
| 2.107 | C≡CH | Me | H | |
| 2.108 | CH₂CH═CH₂ | Me | H | |
| 2.109 | CHFCH₃ | Me | H | |
| 2.110 | CH₂OH | Me | H | |
| 2.111 | CH₂OCH₃ | Me | H | |
| 2.112 | CH₂OCOCH₃ | Me | H | |
| 2.113 | CO₂Me | Me | H | |
| 2.114 | CH₂SMe | Me | H | |
| 2.115 | CH₂SO₂Me | Me | H | |
| 2.116 | CH₂—O—CH₂-(2-tetrahydrofuryl) | Me | H | |
| 2.117 | NH₂ | Me | H | |
| 2.118 | N(CH₃)₂ | Me | H | |
| 2.119 | NHC(O)Me | Me | H | |
| 2.120 | OCH₃ | Me | H | |
| 2.121 | O(CH₂)₂OMe | Me | H | |
| 2.122 | OCHF₂ | Me | H | |
| 2.123 | OPh | Me | H | |
| 2.124 | 4-Cl-Ph | Me | H | |
| 2.125 | SH | Me | H | |
| 2.126 | SCH₃ | Me | H | |
| 2.127 | SO₂CH₃ | Me | H | |
| 2.128 | SO₂N(CH₃)₂ | Me | H | |
| 2.129 | SO₂Ph | Me | H | |
| 2.130 | 3-pyridyl | Me | H | |
| 2.131 | Br | CHFCH₃ | H | |
| 2.132 | CH₃ | CHFCH₃ | H | |
| 2.133 | CH(CH₃)₂ | CHFCH₃ | H | |
| 2.134 | cyclopropyl | CHFCH₃ | H | |
| 2.135 | C≡CH | CHFCH₃ | H | |
| 2.136 | CH₂CH═CH₂ | CHFCH₃ | H | |
| 2.137 | CHFCH₃ | CHFCH₃ | H | |
| 2.138 | CH₂OCH₃ | CHFCH₃ | H | |
| 2.139 | CH₂OCOCH₃ | CHFCH₃ | H | |
| 2.140 | CH₂SMe | CHFCH₃ | H | |
| 2.141 | NH₂ | CHFCH₃ | H | |
| 2.142 | N(CH₃)₂ | CHFCH₃ | H | |
| 2.143 | NHC(O)Me | CHFCH₃ | H | |
| 2.144 | OCH₃ | CHFCH₃ | H | |
| 2.145 | O(CH₂)₂OMe | CHFCH₃ | H | |
| 2.146 | OCHF₂ | CHFCH₃ | H | |
| 2.147 | 4-Cl-Ph | CHFCH₃ | H | |
| 2.148 | SCH₃ | CHFCH₃ | H | |
| 2.149 | SO₂CH₃ | CHFCH₃ | H | |
| 2.150 | 3-pyridyl | CHFCH₃ | H | |
| 2.151 | Br | CF(CH₃)₂ | H | |
| 2.152 | CH₃ | CF(CH₃)₂ | H | |
| 2.153 | CH(CH₃)₂ | CF(CH₃)₂ | H | |
| 2.154 | cyclopropyl | CF(CH₃)₂ | H | |
| 2.155 | C≡CH | CF(CH₃)₂ | H | |
| 2.156 | CH₂CH═CH₂ | CF(CH₃)₂ | H | |
| 2.157 | CHFCH₃ | CF(CH₃)₂ | H | |
| 2.158 | CH₂OCH₃ | CF(CH₃)₂ | H | |
| 2.159 | CH₂SMe | CF(CH₃)₂ | H | |
| 2.160 | N(CH₃)₂ | CF(CH₃)₂ | H | |
| 2.161 | NHC(O)Me | CF(CH₃)₂ | H | |
| 2.162 | OCH₃ | CF(CH₃)₂ | H | |
| 2.163 | O(CH₂)₂OMe | CF(CH₃)₂ | H | |
| 2.164 | OCHF₂ | CF(CH₃)₂ | H | |
| 2.165 | 4-Cl-Ph | CF(CH₃)₂ | H | |
| 2.166 | SCH₃ | CF(CH₃)₂ | H | |
| 2.167 | 3-pyridyl | CF(CH₃)₂ | H | |
| 2.168 | Br | CN | H | |
| 2.169 | CH₃ | CN | H | |
| 2.170 | CH₂OCH₃ | CN | H | |
| 2.171 | CH₂SMe | CN | H | |
| 2.172 | N(CH₃)₂ | CN | H | |
| 2.173 | OCH₃ | CN | H | |
| 2.174 | OCHF₂ | CN | H | |
| 2.175 | 4-Cl-Ph | CN | H | |
| 2.176 | SCH₃ | CN | H | |
| 2.177 | 3-pyridyl | CN | H | |
| 2.178 | Br | triazolyl | H | |
| 2.179 | CH₃ | triazolyl | H | |
| 2.180 | Br | CF₃ | Me | |
| 2.181 | CH₃ | CF₃ | Me | |
| 2.182 | CH(CH₃)₂ | CF₃ | Me | |
| 2.183 | cyclopropyl | CF₃ | Me | |
| 2.184 | C≡CH | CF₃ | Me | |
| 2.185 | CH₂CH═CH₂ | CF₃ | Me | |
| 2.186 | CHFCH₃ | CF₃ | Me | |
| 2.187 | CH₂OCH₃ | CF₃ | Me | |
| 2.188 | CH₂SMe | CF₃ | Me | |
| 2.189 | N(CH₃)₂ | CF₃ | Me | |
| 2.190 | NHC(O)Me | CF₃ | Me | |
| 2.191 | OCH₃ | CF₃ | Me | |
| 2.192 | O(CH₂)₂OMe | CF₃ | Me | |
| 2.193 | OCHF₂ | CF₃ | Me | |
| 2.194 | 4-Cl-Ph | CF₃ | Me | |
| 2.195 | SCH₃ | CF₃ | Me | |
| 2.196 | 3-pyridyl | CF₃ | Me | |
| 2.197 | Br | CF₃ | Cl | |
| 2.198 | CH₃ | CF₃ | Cl | |
| 2.199 | CH₂OCH₃ | CF₃ | Cl | |
| 2.200 | CH₂SMe | CF₃ | Cl | |
| 2.201 | N(CH₃)₂ | CF₃ | Cl | |
| 2.202 | OCH₃ | CF₃ | Cl | |
| 2.203 | OCHF₂ | CF₃ | Cl | |
| 2.204 | 4-Cl-Ph | CF₃ | Cl | |
| 2.205 | SCH₃ | CF₃ | Cl | |
| 2.206 | 3-pyridyl | CF₃ | Cl | |
| 2.207 | Br | CF₃ | OMe | |
| 2.208 | CH₃ | CF₃ | OMe | |
| 2.209 | OEt | CF₃ | H | 1.60 min; 396, 394 |

TABLE 3
Compounds of formula I-1b:

| | |
|---|---|
| | (I-1b) |
| 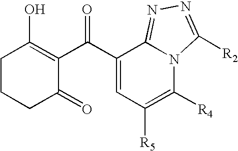 | |

| Cmpd No | R₂ | R₄ | R₅ | Phys. data |
|---|---|---|---|---|
| 3.1 | H | CF₃ | H | 0.91 min; 326, 324 |
| 3.2 | Cl | CF₃ | H | 1.22 min; 362, 360 |
| 3.3 | Br | CF₃ | H | |
| 3.4 | CH₃ | CF₃ | H | ¹H-NMR (CDCl₃, ppm): 7.42 (d, 1 H), 7.25 (d, 1 H), 7.01 (d, 1 H), |
| | | | | 2.91 (m, 3 H), 2.81 (m, 2 H), 2.44, (m, 2 H), 2.13 (m, 2 H). |
| 3.5 | CH₂CH₃ | CF₃ | H | 1.19 min; 354, 352 |
| 3.6 | CH(CH₃)₂ | CF₃ | H | 1.32 min; 368, 366 |
| 3.7 | cyclopropyl | CF₃ | H | 1.24 min; 366; 364 |
| 3.8 | CH₂OCH₃ | CF₃ | H | 1.06 min; 370, 368 |
| 3.9 | CH₂SMe | CF₃ | H | |
| 3.10 | N(CH₃)₂ | CF₃ | H | |
| 3.11 | OCH₃ | CF₃ | H | |
| 3.12 | OCHF₂ | CF₃ | H | |
| 3.13 | 4-Cl-Ph | CF₃ | H | 1.50 min; 436, 434 |
| 3.14 | SCH₃ | CF₃ | H | |
| 3.15 | 3-pyridyl | CF₃ | H | |
| 3.16 | H | CF₂H | H | |
| 3.17 | CH₃ | CF₂H | H | 0.92 min; 322, 320 |

Table 4: Compounds of Formula I-1c:

TABLE 4
Compounds of formula I-1c:

| | |
|---|---|
| | (I-1c) |
| 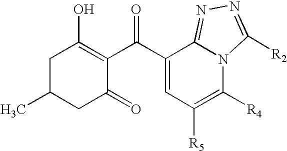 | |

| Cmpd No | R₂ | R₄ | R₅ | Phys. data |
|---|---|---|---|---|
| 4.1 | H | CF₃ | H | |
| 4.2 | Br | CF₃ | H | |
| 4.3 | CH₃ | CF₃ | H | 1.22 min; 354, 352 |
| 4.4 | CH₂OCH₃ | CF₃ | H | |
| 4.5 | CH₂SMe | CF₃ | H | |
| 4.6 | N(CH₃)₂ | CF₃ | H | |
| 4.7 | OCH₃ | CF₃ | H | |
| 4.8 | OCHF₂ | CF₃ | H | |
| 4.9 | 4-Cl-Ph | CF₃ | H | |
| 4.10 | SCH₃ | CF₃ | H | |
| 4.11 | 3-pyridyl | CF₃ | H | |

TABLE 5
Compounds of formula I-1d:

| | |
|---|---|
| | (I-1d) |
| 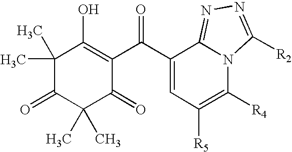 | |

| Cmpd No | R₂ | R₄ | R₅ | Phys. data |
|---|---|---|---|---|
| 5.1 | H | CF₃ | H | solid |
| 5.2 | Br | CF₃ | H | |
| 5.3 | CH₃ | CF₃ | H | 1.34 min; 410, 408 |
| 5.4 | CH(CH₃)₂ | CF₃ | H | 1.52 min; 438, 436 |
| 5.5 | CH₂OCH₃ | CF₃ | H | 1.27 min; 440, 438 |
| 5.6 | CH₂SMe | CF₃ | H | |
| 5.7 | N(CH₃)₂ | CF₃ | H | |
| 5.8 | OCH₃ | CF₃ | H | |
| 5.9 | OCHF₂ | CF₃ | H | |
| 5.10 | 4-Cl-Ph | CF₃ | H | 1.64 min; 506, 504 |
| 5.11 | SCH₃ | CF₃ | H | |
| 5.12 | 3-pyridyl | CF₃ | H | |
| 5.13 | Cl | CF₃ | H | 1.36 min; 432, 430 |

TABLE 6
Compounds of formula I-1e:

| | |
|---|---|
| | (I-1e) |
| 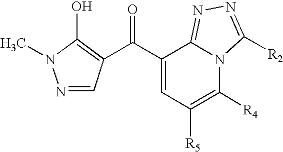 | |

| Cmpd No | R₂ | R₄ | R₅ | Phys. data |
|---|---|---|---|---|
| 6.1 | H | CF₃ | H | |
| 6.2 | Br | CF₃ | H | |
| 6.3 | CH₃ | CF₃ | H | 0.82 min; 326, 324 |
| 6.4 | CH₂OCH₃ | CF₃ | H | |
| 6.5 | CH₂SMe | CF₃ | H | |
| 6.6 | N(CH₃)₂ | CF₃ | H | |
| 6.7 | OCH₃ | CF₃ | H | |
| 6.8 | OCHF₂ | CF₃ | H | |
| 6.9 | 4-Cl-Ph | CF₃ | H | |
| 6.10 | SCH₃ | CF₃ | H | |
| 6.11 | 3-pyridyl | CF₃ | H | |

TABLE 7
Compounds of formula I-1f:

| | |
|---|---|
| | (I-1f) |
| 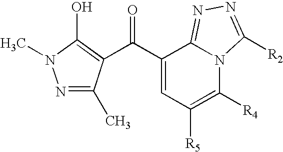 | |

| Cmpd No | R₂ | R₄ | R₅ | Phys. data |
|---|---|---|---|---|
| 7.1 | H | CF₃ | H | |
| 7.2 | Br | CF₃ | H | |
| 7.3 | CH₃ | CF₃ | H | |
| 7.4 | CH₂OCH₃ | CF₃ | H | |
| 7.5 | CH₂SMe | CF₃ | H | |
| 7.6 | N(CH₃)₂ | CF₃ | H | |
| 7.7 | OCH₃ | CF₃ | H | |
| 7.8 | OCHF₂ | CF₃ | H | |
| 7.9 | 4-Cl-Ph | CF₃ | H | |
| 7.10 | SCH₃ | CF₃ | H | |
| 7.11 | 3-pyridyl | CF₃ | H | |

TABLE 8
Compounds of formula I-1g (represented by
formulae I-1ga and I-1gb):

| | |
|---|---|
| | (I-ga) |
| 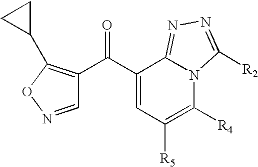 | |
| | (I-gb) |
| 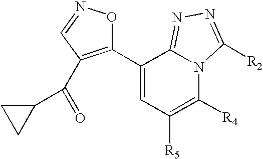 | |

| Cmpd No | R₂ | R₄ | R₅ | Phys. data |
|---|---|---|---|---|
| 8.1 | H | CF₃ | H | |
| 8.2 | Br | CF₃ | H | |
| 8.3 | CH₃ | CF₃ | H | |
| 8.4 | CH₂OCH₃ | CF₃ | H | |
| 8.5 | CH₂SMe | CF₃ | H | |
| 8.6 | N(CH₃)₂ | CF₃ | H | |
| 8.7 | OCH₃ | CF₃ | H | |
| 8.8 | OCHF₂ | CF₃ | H | |
| 8.9 | 4-Cl-Ph | CF₃ | H | |
| 8.10 | SCH₃ | CF₃ | H | |
| 8.11 | 3-pyridyl | CF₃ | H | |
| 8.13 | Cl | CF₃ | H | 1 H NMR (CDCl₃): Isomer A d 1.33 |
| | | | | (2 H, m); 1.43 (2 H, m); 2.99 (1 H, |
| | | | | m); 7.61 (1 H, d); 7.66 (1 H, d); 8.25 |
| | | | | (1 H, s). Isomer B: 1.08 (2 H, m); |
| | | | | 1.26 (2 H, m); 2.28 (1 H, m); 7.59 |
| | | | | (1 H, d); 7.84 (1 H, d); 8.84 (1 H, s). |

TABLE 9
Compounds of formula I-1h (represented by the formulae
I-1ha and I-1hb):

| | |
|---|---|
| | (I-1ha) |
| 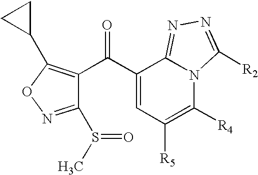 | |
| | (I-1hb) |
| 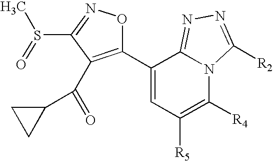 | |

| Cmpd No | R₂ | R₄ | R₅ | Phys. data |
|---|---|---|---|---|
| 9.1 | H | CF₃ | H | |
| 9.2 | Br | CF₃ | H | |
| 9.3 | CH₃ | CF₃ | H | |
| 9.4 | CH₂OCH₃ | CF₃ | H | |
| 9.5 | CH₂SMe | CF₃ | H | |
| 9.6 | N(CH₃)₂ | CF₃ | H | |
| 9.7 | OCH₃ | CF₃ | H | |
| 9.8 | OCHF₂ | CF₃ | H | |
| 9.9 | 4-Cl-Ph | CF₃ | H | |
| 9.10 | SCH₃ | CF₃ | H | |
| 9.11 | 3-pyridyl | CF₃ | H | |
| 9.12 | Cl | CF₃ | H | |

TABLE 10
Compounds of formula I-1i

| | |
|---|---|
| | (I-1i) |
| 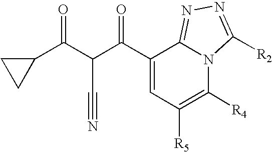 | |

| Cmpd No | R₂ | R₄ | R₅ | Phys. data |
|---|---|---|---|---|
| 10.1 | H | CF₃ | H | |
| 10.2 | Br | CF₃ | H | |
| 10.3 | CH₃ | CF₃ | H | |
| 10.4 | CH₂OCH₃ | CF₃ | H | |
| 10.5 | CH₂SMe | CF₃ | H | |
| 10.6 | N(CH₃)₂ | CF₃ | H | |
| 10.7 | OCH₃ | CF₃ | H | |
| 10.8 | OCHF₂ | CF₃ | H | |
| 10.9 | 4-Cl-Ph | CF₃ | K | |
| 10.10 | SCH₃ | CF₃ | H | |
| 10.11 | 3-pyridyl | CF₃ | H | |
| 10.12 | Cl | CF₃ | H | 1 H NMR (CDCl₃) d 1.39 (2 H, |
| | | | | m); 1.53 (2 H, m); 2.42 (1 H, m); |
| | | | | 7.58 (1 H, d); 7.67 (1 H, d). |

TABLE 11
Compounds of formula I-2b:

| | |
|---|---|
| | (I-2b) |
| 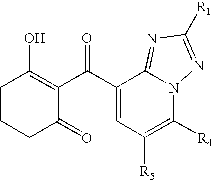 | |

| Cmpd No | R₁ | R₄ | R₅ | Physchem data |
|---|---|---|---|---|
| 11.1 | H | CF₃ | H | |
| 11.2 | Br | CF₃ | H | |
| 11.3 | CH₃ | CF₃ | H | 1.17 min; 340, 338 |
| 11.4 | CH₂OCH₃ | CF₃ | H | |
| 11.5 | CH₂SMe | CF₃ | H | |
| 11.6 | N(CH₃)₂ | CF₃ | H | |
| 11.7 | OCH₃ | CF₃ | H | |
| 11.8 | OCHF₂ | CF₃ | H | |
| 11.9 | 4-Cl-Ph | CF₃ | H | |
| 11.10 | SCH₃ | CF₃ | H | |
| 11.11 | 3-pyridyl | CF₃ | H | |

TABLE 12
Compounds of formula I-2c:

| | |
|---|---|
| | (I-2c) |
| 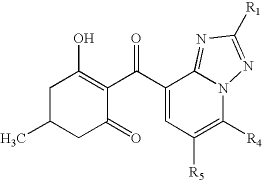 | |

| Cmpd No | R₁ | R₄ | R₅ | Physchem data |
|---|---|---|---|---|
| 12.1 | H | CF₃ | H | |
| 12.2 | Br | CF₃ | H | |
| 12.3 | CH₃ | CF₃ | H | |
| 12.4 | CH₂OCH₃ | CF₃ | H | |
| 12.5 | CH₂SMe | CF₃ | H | |
| 12.6 | N(CH₃)₂ | CF₃ | H | |
| 12.7 | OCH₃ | CF₃ | H | |
| 12.8 | OCHF₂ | CF₃ | H | |
| 12.9 | 4-Cl-Ph | CF₃ | H | |
| 12.10 | SCH₃ | CF₃ | H | |
| 12.11 | 3-pyridyl | CF₃ | H | |

TABLE 13
Compounds of formula I-2d:

| | |
|---|---|
| | (I-2d) |
| 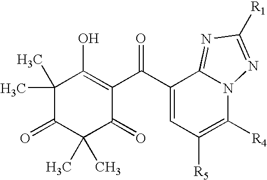 | |

| Cmpd No | R₁ | R₄ | R₅ | Phys. data |
|---|---|---|---|---|
| 13.1 | H | CF₃ | H | 1.13 min; 396, 394 |
| 13.2 | Br | CF₃ | H | |
| 13.3 | CH₃ | CF₃ | H | 1.51 min; 410, 408 |
| 13.4 | CH₂OCH₃ | CF₃ | H | |
| 13.5 | CH₂SMe | CF₃ | H | |
| 13.6 | N(CH₃)₂ | CF₃ | H | |
| 13.7 | OCH₃ | CF₃ | H | |
| 13.8 | OCHF₂ | CF₃ | H | |
| 13.9 | 4-Cl-Ph | CF₃ | H | |
| 13.10 | SCH₃ | CF₃ | H | |
| 13.11 | 3-pyridyl | CF₃ | H | |

TABLE 14
Compounds of formula I-2e:

| | |
|---|---|
| | (I-2e) |
| 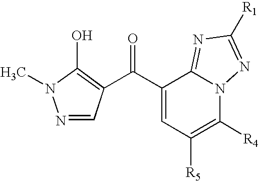 | |

| Cmpd No | R₁ | R₄ | R₅ | Phys. data |
|---|---|---|---|---|
| 14.1 | H | CF₃ | H | |
| 14.2 | Br | CF₃ | H | |
| 14.3 | CH₃ | CF₃ | H | |
| 14.4 | CH₂OCH₃ | CF₃ | H | |
| 14.5 | CH₂SMe | CF₃ | H | |
| 14.6 | N(CH₃)₂ | CF₃ | H | |
| 14.7 | OCH₃ | CF₃ | H | |
| 14.8 | OCHF₂ | CF₃ | H | |
| 14.9 | 4-Cl-Ph | CF₃ | H | |
| 14.10 | SCH₃ | CF₃ | H | |
| 14.11 | 3-pyridyl | CF₃ | H | |

TABLE 15
Compounds of formula I-2f:

| | |
|---|---|
| | (I-2f) |
| 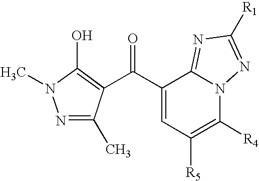 | |

| Cmpd No | R₁ | R₄ | R₅ | Phys. data |
|---|---|---|---|---|
| 15.1 | H | CF₃ | H | |
| 15.2 | Br | CF₃ | H | |
| 15.3 | CH₃ | CF₃ | H | |
| 15.4 | CH₂OCH₃ | CF₃ | H | |
| 15.5 | CH₂SMe | CF₃ | H | |
| 15.6 | N(CH₃)₂ | CF₃ | H | |
| 15.7 | OCH₃ | CF₃ | H | |
| 15.8 | OCHF₂ | CF₃ | H | |
| 15.9 | 4-Cl-Ph | CF₃ | H | |
| 15.10 | SCH₃ | CF₃ | H | |
| 15.11 | 3-pyridyl | CF₃ | H | |

TABLE 16
Compounds of formula I-2g (represented by the
formulae I-2ga and I-2gb):

| | |
|---|---|
| | (I-2ga) |
| 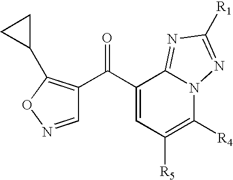 | |
| | (I-2gb) |
| 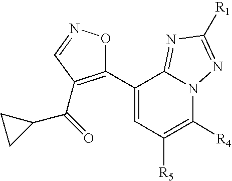 | |

| Cmpd No | R₁ | R₄ | R₅ | Phys. data |
|---|---|---|---|---|
| 16.1 | H | CF₃ | H | |
| 16.2 | Br | CF₃ | H | |
| 16.3 | CH₃ | CF₃ | H | |
| 16.4 | CH₂OCH₃ | CF₃ | H | |
| 16.5 | CH₂SMe | CF₃ | H | |
| 16.6 | N(CH₃)₂ | CF₃ | H | |
| 16.7 | OCH₃ | CF₃ | H | |
| 16.8 | OCHF₂ | CF₃ | H | |
| 16.9 | 4-Cl-Ph | CF₃ | H | |
| 16.10 | SCH₃ | CF₃ | H | |
| 16.11 | 3-pyridyl | CF₃ | H | |

TABLE 17
Compounds of formula I-2h (represented by the
formulae I-2ha and I-2hb):

| | |
|---|---|
| | (I-2ha) |
| 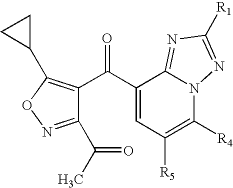 | |
| | (I-2hb) |
| 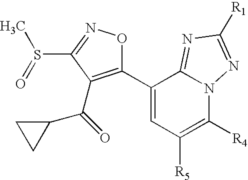 | |

| Cmpd No | R₁ | R₄ | R₅ | Phys. data |
|---|---|---|---|---|
| 17.1 | H | CF₃ | H | |
| 17.2 | Br | CF₃ | H | |
| 17.3 | CH₃ | CF₃ | H | |
| 17.4 | CH₂OCH₃ | CF₃ | H | |
| 17.5 | CH₂SMe | CF₃ | H | |
| 17.6 | N(CH₃)₂ | CF₃ | H | |
| 17.7 | OCH₃ | CF₃ | H | |
| 17.8 | OCHF₂ | CF₃ | H | |
| 17.9 | 4-Cl-Ph | CF₃ | H | |
| 17.10 | SCH₃ | CF₃ | H | |
| 17.11 | 3-pyridyl | CF₃ | H | |

TABLE 18
Compounds of formula I-2i:

| | |
|---|---|
| | (I-2i) |
| 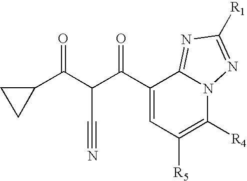 | |

| Cmpd No | R₁ | R₄ | R₅ | Physchem data |
|---|---|---|---|---|
| 18.1 | H | CF₃ | H | |
| 18.2 | Br | CF₃ | H | |
| 18.3 | CH₃ | CF₃ | H | |
| 18.4 | CH₂OCH₃ | CF₃ | H | |
| 18.5 | CH₂SMe | CF₃ | H | |
| 18.6 | N(CH₃)₂ | CF₃ | H | |
| 18.7 | OCH₃ | CF₃ | H | |
| 18.8 | OCHF₂ | CF₃ | H | |
| 18.9 | 4-Cl-Ph | CF₃ | H | |
| 18.10 | SCH₃ | CF₃ | H | |
| 18.11 | 3-pyridyl | CF₃ | H | |

TABLE 19
Compounds of formula I-3a:

| | |
|---|---|
| | (I-3a) |
| 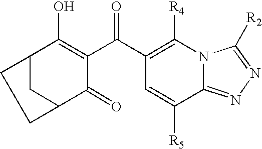 | |

| Cmpd No | R₂ | R₄ | R₅ | Phys. data |
|---|---|---|---|---|
| 19.1 | H | CH₃ | H | |
| 19.2 | Cl | CH₃ | H | |
| 19.3 | Br | CH₃ | H | |
| 19.4 | CH₃ | CH₃ | H | |
| 19.5 | CH₂CH₃ | CH₃ | H | |
| 19.6 | (CH₂)₅CH₃ | CH₃ | H | |
| 19.7 | CH(CH₃)₂ | CH₃ | H | |
| 19.8 | cyclopropyl | CH₃ | H | |
| 19.9 | CCH | CH₃ | H | |
| 19.10 | CH═CH₂ | CH₃ | H | |
| 19.11 | CH₂CH═CH₂ | CH₃ | H | |
| 19.12 | CH₂F | CH₃ | H | |
| 19.13 | CHFCH₃ | CH₃ | H | |
| 19.14 | CHF₂ | CH₃ | H | |
| 19.15 | CH₂OH | CH₃ | H | |
| 19.16 | CH₂OCH₃ | CH₃ | H | |
| 19.17 | CH₂CH₂OCH₃ | CH₃ | H | |
| 19.18 | CH₂OCOCH₃ | CH₃ | H | |
| 19.19 | CH₂OCOPh | CH₃ | H | |
| 19.20 | CO₂Me | CH₃ | H | |
| 19.21 | CH₂SMe | CH₃ | H | |
| 19.22 | CH₂SO₂Me | CH₃ | H | |
| 19.23 | CH₂SO₂Ph | CH₃ | H | |
| 19.24 | CH₂SOMe | CH₃ | H | |
| 19.25 | CH₂—O—CH₂-(2-tetrahydrofuryl) | CH₃ | H | |
| 19.26 | NH2 | CH₃ | H | |
| 19.27 | N(CH₃)₂ | CH₃ | H | |
| 19.28 | NHC(O)Me | CH₃ | H | |
| 19.29 | OCH₃ | CH₃ | H | |
| 19.30 | OCH₂CCH | CH₃ | H | |
| 19.31 | O(CH₂)₂OMe | CH₃ | H | |
| 19.32 | OCHF₂ | CH₃ | H | |
| 19.33 | OPh | CH₃ | H | |
| 19.34 | Ph | CH₃ | H | |
| 19.35 | 4-Cl-Ph | CH₃ | H | |
| 19.36 | SH | CH₃ | H | |
| 19.37 | SCH₂Ph | CH₃ | H | |
| 19.38 | SCH₃ | CH₃ | H | |
| 19.39 | SO₂CH₂Ph | CH₃ | H | |
| 19.40 | SO₂CH₃ | CH₃ | H | |
| 19.41 | SO₂N(CH₃)₂ | CH₃ | H | |
| 19.42 | SO₂Ph | CH₃ | H | |
| 19.43 | SOCH₂Ph | CH₃ | H | |
| 19.44 | SOCH₃ | CH₃ | H | |
| 19.45 | SOPh | CH₃ | H | |
| 19.46 | SPh | CH₃ | H | |
| 19.47 | 2-furyl | CH₃ | H | |
| 19.48 | 2-pyridyl | CH₃ | H | |
| 19.49 | 3-pyridyl | CH₃ | H | |
| 19.50 | 4-pyridyl | CH₃ | H | |
| 19.51 | H | CF₂H | H | |
| 19.52 | Cl | CF₂H | H | |
| 19.53 | Br | CF₂H | H | |
| 19.54 | CH₃ | CF₂H | H | |
| 19.55 | CH₂CH₃ | CF₂H | H | |
| 19.56 | (CH₂)₅CH₃ | CF₂H | H | |
| 19.57 | CH(CH₃)₂ | CF₂H | H | |
| 19.58 | cyclopropyl | CF₂H | H | |
| 19.59 | CCH | CF₂H | H | |
| 19.60 | CH═CH₂ | CF₂H | H | |
| 19.61 | CH₂CH═CH₂ | CF₂H | H | |
| 19.62 | CH₂F | CF₂H | H | |
| 19.63 | CHFCH₃ | CF₂H | H | |
| 19.64 | CHF₂ | CF₂H | H | |
| 19.65 | CH₂OH | CF₂H | H | |
| 19.66 | CH₂OCH₃ | CF₂H | H | |
| 19.67 | CH₂CH₂OCH₃ | CF₂H | H | |
| 19.68 | CH₂OCOCH₃ | CF₂H | H | |
| 19.69 | CH₂OCOPh | CF₂H | H | |
| 19.70 | CO₂Me | CF₂H | H | |
| 19.71 | CH₂SMe | CF₂H | H | |
| 19.72 | CH₂SO₂Me | CF₂H | H | |
| 19.73 | CH₂SO₂Ph | CF₂H | H | |
| 19.74 | CH₂SOMe | CF₂H | H | |
| 19.75 | CH₂—O—CH₂-(2-tetrahydrofuryl) | CF₂H | H | |
| 19.76 | NH₂ | CF₂H | H | |
| 19.77 | N(CH₃)₂ | CF₂H | H | |
| 19.78 | NHC(O)Me | CF₂H | H | |
| 19.79 | OCH₃ | CF₂H | H | |
| 19.80 | OCH₂CCH | CF₂H | H | |
| 19.81 | O(CH₂)₂OMe | CF₂H | H | |
| 19.82 | OCHF₂ | CF₂H | H | |
| 19.83 | OPh | CF₂H | H | |
| 19.84 | Ph | CF₂H | H | |
| 19.85 | 4-Cl-Ph | CF₂H | H | |
| 19.86 | SH | CF₂H | H | |
| 19.87 | SCH₂Ph | CF₂H | H | |
| 19.88 | SCH₃ | CF₂H | H | |
| 19.89 | SO₂CH₂Ph | CF₂H | H | |
| 19.90 | SO₂CH₃ | CF₂H | H | |
| 19.91 | SO₂N(CH₃)₂ | CF₂H | H | |
| 19.92 | SO₂Ph | CF₂H | H | |
| 19.93 | SOCH₂Ph | CF₂H | H | |
| 19.94 | SOCH₃ | CF₂H | H | |
| 19.95 | SOPh | CF₂H | H | |
| 19.96 | SPh | CF₂H | H | |
| 19.97 | 2-furyl | CF₂H | H | |
| 19.98 | 2-pyridyl | CF₂H | H | |
| 19.99 | 3-pyridyl | CF₂H | H | |
| 19.100 | 4-pyridyl | CF₂H | H | |

TABLE 20
Compounds of formula I-4a:

| | |
|---|---|
| | (I-4a) |
| 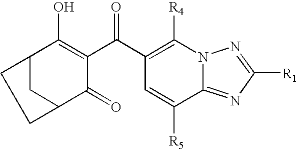 | |

| Cmpd No | R₁ | R₄ | R₅ | Phys. data |
|---|---|---|---|---|
| 20.1 | H | CH₃ | H | |
| 20.2 | Cl | CH₃ | H | |
| 20.3 | Br | CH₃ | H | |
| 20.4 | CH₃ | CH₃ | H | |
| 20.5 | CH₂CH₃ | CH₃ | H | |
| 20.6 | (CH₂)₅CH₃ | CH₃ | H | |
| 20.7 | CH(CH₃)₂ | CH₃ | H | |
| 20.8 | cyclopropyl | CH₃ | H | |
| 20.9 | CCH | CH₃ | H | |
| 20.10 | CH═CH₂ | CH₃ | H | |
| 20.11 | CH₂CH═CH₂ | CH₃ | H | |
| 20.12 | CH₂F | CH₃ | H | |
| 20.13 | CHFCH₃ | CH₃ | H | |
| 20.14 | CHF₂ | CH₃ | H | |
| 20.15 | CH₂OH | CH₃ | H | |
| 20.16 | CH₂OCH₃ | CH₃ | H | |
| 20.17 | CH₂CH₂OCH₃ | CH₃ | H | |
| 20.18 | CH₂OCOCH₃ | CH₃ | H | |
| 20.19 | CH₂OCOPh | CH₃ | H | |
| 20.20 | CO₂Me | CH₃ | H | |
| 20.21 | CH₂SMe | CH₃ | H | |
| 20.22 | CH₂SO₂Me | CH₃ | H | |
| 20.23 | CH₂SO₂Ph | CH₃ | H | |
| 20.24 | CH₂SOMe | CH₃ | H | |
| 20.25 | CH₂—O—CH₂-(2-tetrahydrofuryl) | CH₃ | H | |
| 20.26 | NH₂ | CH₃ | H | |
| 20.27 | N(CH₃)₂ | CH₃ | H | |
| 20.28 | NHC(O)Me | CH₃ | H | |
| 20.29 | OCH₃ | CH₃ | H | |
| 20.30 | OCH₂CCH | CH₃ | H | |
| 20.31 | O(CH₂)₂OMe | CH₃ | H | |
| 20.32 | OCHF₂ | CH₃ | H | |
| 20.33 | OPh | CH₃ | H | |
| 20.34 | Ph | CH₃ | H | |
| 20.35 | 4-Cl-Ph | CH₃ | H | |
| 20.36 | SH | CH₃ | H | |
| 20.37 | SCH₂Ph | CH₃ | H | |
| 20.38 | SCH₃ | CH₃ | H | |
| 20.39 | SO₂CH₂Ph | CH₃ | H | |
| 20.40 | SO₂CH₃ | CH₃ | H | |
| 20.41 | SO₂N(CH₃)₂ | CH₃ | H | |
| 20.42 | SO₂Ph | CH₃ | H | |
| 20.43 | SOCH₂Ph | CH₃ | H | |
| 20.44 | SOCH₃ | CH₃ | H | |
| 20.45 | SOPh | CH₃ | H | |
| 20.46 | SPh | CH₃ | H | |
| 20.47 | 2-furyl | CH₃ | H | |
| 20.48 | 2-pyridyl | CH₃ | H | |
| 20.49 | 3-pyridyl | CH₃ | H | |
| 20.50 | 4-pyridyl | CH₃ | H | |
| 20.51 | H | CF₂H | H | |
| 20.52 | Cl | CF₂H | H | |
| 20.53 | Br | CF₂H | H | |
| 20.54 | CH₃ | CF₂H | H | |
| 20.55 | CH₂CH₃ | CF₂H | H | |
| 20.56 | (CH₂)5CH₃ | CF₂H | H | |
| 20.57 | CH(CH₃)₂ | CF₂H | H | |
| 20.58 | cyclopropyl | CF₂H | H | |
| 20.59 | C≡CH | CF₂H | H | |
| 20.60 | CH═CH₂ | CF₂H | H | |
| 20.61 | CH₂CH═CH₂ | CF₂H | H | |
| 20.62 | CH₂F | CF₂H | H | |
| 20.63 | CHFCH₃ | CF₂H | H | |
| 20.64 | CHF₂ | CF₂H | H | |
| 20.65 | CH₂OH | CF₂H | H | |
| 20.66 | CH₂OCH₃ | CF₂H | H | |
| 20.67 | CH₂CH₂OCH₃ | CF₂H | H | |
| 20.68 | CH₂OCOCH₃ | CF₂H | H | |
| 20.69 | CH₂OCOPh | CF₂H | H | |
| 20.70 | CO₂Me | CF₂H | H | |
| 20.71 | CH₂SMe | CF₂H | H | |
| 20.72 | CH₂SO₂Me | CF₂H | H | |
| 20.73 | CH₂SO₂Ph | CF₂H | H | |
| 20.74 | CH₂SOMe | CF₂H | H | |
| 20.75 | CH₂—O—CH₂-(2-tetrahydrofuryl) | CF₂H | H | |
| 20.76 | NH₂ | CF₂H | H | |
| 20.77 | N(CH₃)₂ | CF₂H | H | |
| 20.78 | NHC(O)Me | CF₂H | H | |
| 20.79 | OCH₃ | CF₂H | H | |
| 20.80 | OCH₂CCH | CF₂H | H | |
| 20.81 | O(CH₂)₂OMe | CF₂H | H | |
| 20.82 | OCHF₂ | CF₂H | H | |
| 20.83 | OPh | CF₂H | H | |
| 20.84 | Ph | CF₂H | H | |
| 20.85 | 4-Cl-Ph | CF₂H | H | |
| 20.86 | SH | CF₂H | H | |
| 20.87 | SCH₂Ph | CF₂H | H | |
| 20.88 | SCH₃ | CF₂H | H | |
| 20.89 | SO₂CH₂Ph | CF₂H | H | |
| 20.90 | SO₂CH₃ | CF₂H | H | |
| 20.91 | SO₂N(CH₃)₂ | CF₂H | H | |
| 20.92 | SO₂Ph | CF₂H | H | |
| 20.93 | SOCH₂Ph | CF₂H | H | |
| 20.94 | SOCH₃ | CF₂H | H | |
| 20.95 | SOPh | CF₂H | H | |
| 20.96 | SPh | CF₂H | H | |
| 20.97 | 2-furyl | CF₂H | H | |
| 20.98 | 2-pyridyl | CF₂H | H | |
| 20.99 | 3-pyridyl | CF₂H | H | |
| 20.100 | 4-pyridyl | CF₂H | H | |

TABLE 21
Compounds of formula I-3b:

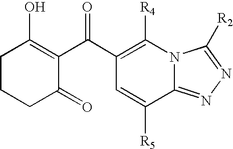

| Cmpd No | R₂ | R₄ | R₅ | Phys. data |
|---|---|---|---|---|
| 21.1 | H | CH₃ | H | |
| 21.2 | Br | CH₃ | H | |
| 21.3 | CH₃ | CH₃ | H | |
| 21.4 | CH₂OCH₃ | CH₃ | H | |
| 21.5 | CH₂SMe | CH₃ | H | |
| 21.6 | N(CH₃)₂ | CH₃ | H | |
| 21.7 | OCH₃ | CH₃ | H | |
| 21.8 | OCHF₂ | CH₃ | H | |
| 21.9 | 4-Cl-Ph | CH₃ | H | |
| 21.10 | SCH₃ | CH₃ | H | |
| 21.11 | 3-pyridyl | CH₃ | H | |

TABLE 22
Compounds of formula I-3c:

| | |
|---|---|
| | (I-3c) |
| 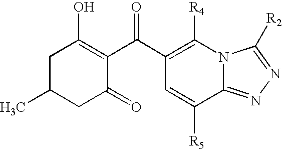 | |

| Cmpd No | R₂ | R₄ | R₅ | Phys. data |
|---|---|---|---|---|
| 22.1 | H | CH₃ | H | |
| 22.2 | Br | CH₃ | H | |
| 22.3 | CH₃ | CH₃ | H | |
| 22.4 | CH₂OCH₃ | CH₃ | H | |
| 22.5 | CH₂SMe | CH₃ | H | |
| 22.6 | N(CH₃)₂ | CH₃ | H | |
| 22.7 | OCH₃ | CH₃ | H | |
| 22.8 | OCHF₂ | CH₃ | H | |
| 22.9 | 4-Cl-Ph | CH₃ | H | |
| 22.10 | SCH₃ | CH₃ | H | |
| 22.11 | 3-pyridyl | CH₃ | H | |

TABLE 23
Compounds of formula I-3d:

| | |
|---|---|
| | (I-3d) |
| 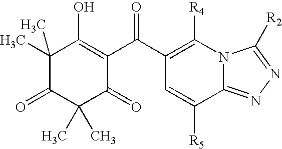 | |

| Cmpd No | R₂ | R₄ | R₅ | Phys. data |
|---|---|---|---|---|
| 23.1 | H | CH₃ | H | |
| 23.2 | Br | CH₃ | H | |
| 23.3 | CH₃ | CH₃ | H | |
| 23.4 | CH₂OCH₃ | CH₃ | H | |
| 23.5 | CH₂SMe | CH₃ | H | |
| 23.6 | N(CH₃)₂ | CH₃ | H | |
| 23.7 | OCH₃ | CH₃ | H | |
| 23.8 | OCHF₂ | CH₃ | H | |
| 23.9 | 4-Cl-Ph | CH₃ | H | |
| 23.10 | SCH₃ | CH₃ | H | |
| 23.11 | 3-pyridyl | CH₃ | H | |

TABLE 24
Compounds of formula I-3e:

| | |
|---|---|
| | (I-3e) |
| 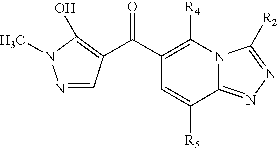 | |

| Cmpd No | R₂ | R₄ | R₅ | Phys. data |
|---|---|---|---|---|
| 24.1 | H | CH₃ | H | |
| 24.2 | Br | CH₃ | H | |
| 24.3 | CH₃ | CH₃ | H | |
| 24.4 | CH₂OCH₃ | CH₃ | H | |
| 24.5 | CH₂SMe | CH₃ | H | |
| 24.6 | N(CH₃)₂ | CH₃ | H | |
| 24.7 | OCH₃ | CH₃ | H | |
| 24.8 | OCHF₂ | CH₃ | H | |
| 24.9 | 4-Cl-Ph | CH₃ | H | |
| 24.10 | SCH₃ | CH₃ | H | |
| 24.11 | 3-pyridyl | CH₃ | H | |

TABLE 25
Compounds of formula I-3f:

| | |
|---|---|
| | (I-3f) |
| 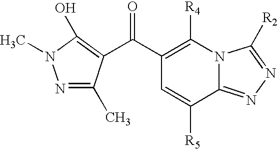 | |

| Cmpd No | R₂ | R₄ | R₅ | Phys. data |
|---|---|---|---|---|
| 25.1 | H | CH₃ | H | |
| 25.2 | Br | CH₃ | H | |
| 25.3 | CH₃ | CH₃ | H | |
| 25.4 | CH₂OCH₃ | CH₃ | H | |
| 25.5 | CH₂SMe | CH₃ | H | |
| 25.6 | N(CH₃)₂ | CH₃ | H | |
| 25.7 | OCH₃ | CH₃ | H | |
| 25.8 | OCHF₂ | CH₃ | H | |
| 25.9 | 4-Cl-Ph | CH₃ | H | |
| 25.10 | SCH₃ | CH₃ | H | |
| 25.11 | 3-pyridyl | CH₃ | H | |

TABLE 26
Compounds of formula I-3g (represented by formulae I-3ga and I-3gb):

| | |
|---|---|
| | (I-3ga) |
| 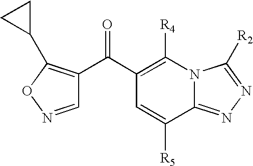 | |
| | (I-3gb) |
| 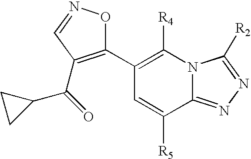 | |

| Cmpd No | R₂ | R₄ | R₅ | Phys. data |
|---|---|---|---|---|
| 26.1 | H | CH₃ | H | |
| 26.2 | Br | CH₃ | H | |
| 26.3 | CH₃ | CH₃ | H | |
| 26.4 | CH₂OCH₃ | CH₃ | H | |
| 26.5 | CH₂SMe | CH₃ | H | |
| 26.6 | N(CH₃)₂ | CH₃ | H | |
| 26.7 | OCH₃ | CH₃ | H | |
| 26.8 | OCHF₂ | CH₃ | H | |
| 26.9 | 4-Cl-Ph | CH₃ | H | |
| 26.10 | SCH₃ | CH₃ | H | |
| 26.11 | 3-pyridyl | CH₃ | H | |

TABLE 27
Compounds of formula I-3h (represented by formulae
(I-3ha and I-3hb):

| | |
|---|---|
| | (I-3ha) |
| 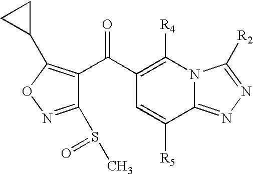 | |
| | (I-3hb) |
| 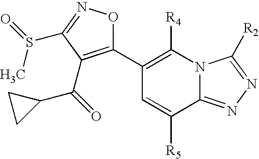 | |

| Cmpd No | R₂ | R₄ | R₅ | Phys. data |
|---|---|---|---|---|
| 27.1 | H | CH₃ | H | |
| 27.2 | Br | CH₃ | H | |
| 27.3 | CH₃ | CH₃ | H | |
| 27.4 | CH₂OCH₃ | CH₃ | H | |
| 27.5 | CH₂SMe | CH₃ | H | |
| 27.6 | N(CH₃)₂ | CH₃ | H | |
| 27.7 | OCH₃ | CH₃ | H | |
| 27.8 | OCHF₂ | CH₃ | H | |
| 27.9 | 4-Cl-Ph | CH₃ | H | |
| 27.10 | SCH₃ | CH₃ | H | |
| 27.11 | 3-pyridyl | CH₃ | H | |

TABLE 28
Compounds of formula I-3i:

| | |
|---|---|
| | (I-3i) |
| 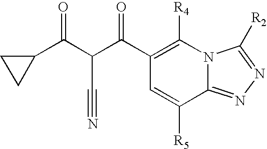 | |

| Cmpd No | R₂ | R₄ | R₅ | Phys. data |
|---|---|---|---|---|
| 28.1 | H | CH₃ | H | |
| 28.2 | Br | CH₃ | H | |
| 28.3 | CH₃ | CH₃ | H | |
| 28.4 | CH₂OCH₃ | CH₃ | H | |
| 28.5 | CH₂SMe | CH₃ | H | |
| 28.6 | N(CH₃)₂ | CH₃ | H | |
| 28.7 | OCH₃ | CH₃ | H | |
| 28.8 | OCHF₂ | CH₃ | H | |
| 28.9 | 4-Cl-Ph | CH₃ | H | |
| 28.10 | SCH₃ | CH₃ | H | |
| 28.11 | 3-pyridyl | CH₃ | H | |

TABLE 29
Compounds of formula I-4b:

| | |
|---|---|
| | (I-4b) |
| 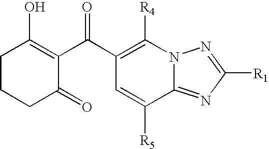 | |

| Cmpd No | R₁ | R₄ | R₅ | Phys. data |
|---|---|---|---|---|
| 29.1 | H | CH₃ | H | |
| 29.2 | Br | CH₃ | H | |
| 29.3 | CH₃ | CH₃ | H | |
| 29.4 | CH₂OCH₃ | CH₃ | H | |
| 29.5 | CH₂SMe | CH₃ | H | |
| 29.6 | N(CH₃)₂ | CH₃ | H | |
| 29.7 | OCH₃ | CH₃ | H | |
| 29.8 | OCHF₂ | CH₃ | H | |
| 29.9 | 4-Cl-Ph | CH₃ | H | |
| 29.10 | SCH₃ | CH₃ | H | |
| 29.11 | 3-pyridyl | CH₃ | H | |

TABLE 30
Compounds of formula I-4c:

| | |
|---|---|
| | (I-4c) |
| 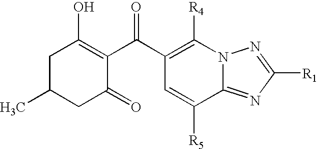 | |

| Cmpd No | R₁ | R₄ | R₅ | Phys. data |
|---|---|---|---|---|
| 30.1 | H | CH₃ | H | |
| 30.2 | Br | CH₃ | H | |
| 30.3 | CH₃ | CH₃ | H | |
| 30.4 | CH₂OCH₃ | CH₃ | H | |
| 30.5 | CH₂SMe | CH₃ | H | |
| 30.6 | N(CH₃)₂ | CH₃ | H | |
| 30.7 | OCH₃ | CH₃ | H | |
| 30.8 | OCHF₂ | CH₃ | H | |
| 30.9 | 4-Cl-Ph | CH₃ | H | |
| 30.10 | SCH₃ | CH₃ | H | |
| 30.11 | 3-pyridyl | CH₃ | H | |

TABLE 31
Compounds of formula I-4d:

| | |
|---|---|
| | (I-4d) |
| 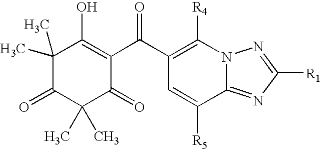 | |

| Cmpd No | R₁ | R₄ | R₅ | Phys. data |
|---|---|---|---|---|
| 31.1 | H | CH₃ | H | |
| 31.2 | Br | CH₃ | H | |
| 31.3 | CH₃ | CH₃ | H | |
| 31.4 | CH₂OCH₃ | CH₃ | H | |
| 31.5 | CH₂SMe | CH₃ | H | |
| 31.6 | N(CH₃)₂ | CH₃ | H | |
| 31.7 | OCH₃ | CH₃ | H | |
| 31.8 | OCHF₂ | CH₃ | H | |
| 31.9 | 4-Cl-Ph | CH₃ | H | |
| 31.10 | SCH₃ | CH₃ | H | |
| 31.11 | 3-pyridyl | CH₃ | H | |

TABLE 32
Compounds of formula I-4e:

| | |
|---|---|
| | (I-4e) |
| 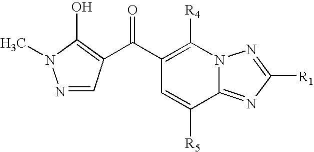 | |

| Cmpd No | R₁ | R₄ | R₅ | Phys. data |
|---|---|---|---|---|
| 32.1 | H | CH₃ | H | |
| 32.2 | Br | CH₃ | H | |
| 32.3 | CH₃ | CH₃ | H | |
| 32.4 | CH₂OCH₃ | CH₃ | H | |
| 32.5 | CH₂SMe | CH₃ | H | |
| 32.6 | N(CH₃)₂ | CH₃ | H | |
| 32.7 | OCH₃ | CH₃ | H | |
| 32.8 | OCHF₂ | CH₃ | H | |
| 32.9 | 4-Cl-Ph | CH₃ | H | |
| 32.10 | SCH₃ | CH₃ | H | |
| 32.11 | 3-pyridyl | CH₃ | H | |

TABLE 33
Compounds of formula I-4f:

| | |
|---|---|
| | (I-4f) |
| 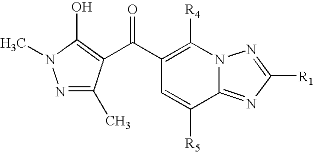 | |

| Cmpd No | R₁ | R₄ | R₅ | Phys. data |
|---|---|---|---|---|
| 33.1 | H | CH₃ | H | |
| 33.2 | Br | CH₃ | H | |
| 33.3 | CH₃ | CH₃ | H | |
| 33.4 | CH₂OCH₃ | CH₃ | H | |
| 33.5 | CH₂SMe | CH₃ | H | |
| 33.6 | N(CH₃)₂ | CH₃ | H | |
| 33.7 | OCH₃ | CH₃ | H | |
| 33.8 | OCHF₂ | CH₃ | H | |
| 33.9 | 4-Cl-Ph | CH₃ | H | |
| 33.10 | SCH₃ | CH₃ | H | |
| 33.11 | 3-pyridyl | CH₃ | H | |

TABLE 34
Compounds of formula I-4g (represented by formulae I-4ga and I-4gb):

| | |
|---|---|
| | (I-4ga) |
| 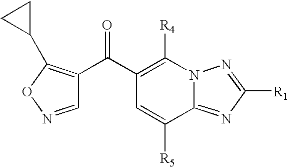 | |
| | (I-4gb) |
| 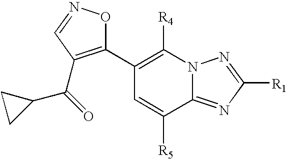 | |

| Cmpd No | R₁ | R₄ | R₅ | Phys. data |
|---|---|---|---|---|
| 34.1 | H | CH₃ | H | |
| 34.2 | Br | CH₃ | H | |
| 34.3 | CH₃ | CH₃ | H | |
| 34.4 | CH₂OCH₃ | CH₃ | H | |
| 34.5 | CH₂SMe | CH₃ | H | |
| 34.6 | N(CH₃)₂ | CH₃ | H | |
| 34.7 | OCH₃ | CH₃ | H | |
| 34.8 | OCHF₂ | CH₃ | H | |
| 34.9 | 4-Cl-Ph | CH₃ | H | |
| 34.10 | SCH₃ | CH₃ | H | |
| 34.11 | 3-pyridyl | CH₃ | H | |

TABLE 35
Compounds of formula I-4h (represented by formulae I-4ha and I-4hb):

| | |
|---|---|
| | (I-4ha) |
| 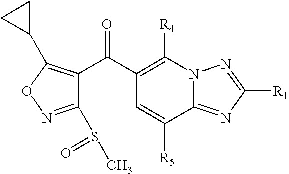 | |
| | (I-4hb) |
| 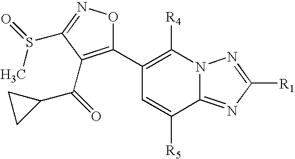 | |

| Cmpd No | R₁ | R₄ | R₅ | Phys. data |
|---|---|---|---|---|
| 35.1 | H | CH₃ | H | |
| 35.2 | Br | CH₃ | H | |
| 35.3 | CH₃ | CH₃ | H | |
| 35.4 | CH₂OCH₃ | CH₃ | H | |
| 35.5 | CH₂SMe | CH₃ | H | |
| 35.6 | N(CH₃)₂ | CH₃ | H | |
| 35.7 | OCH₃ | CH₃ | H | |
| 35.8 | OCHF₂ | CH₃ | H | |
| 35.9 | 4-Cl-Ph | CH₃ | H | |
| 35.10 | SCH₃ | CH₃ | H | |
| 35.11 | 3-pyridyl | CH₃ | H | |

TABLE 36
Compounds of formula I-4i:

| | |
|---|---|
| | (I-4i) |
| 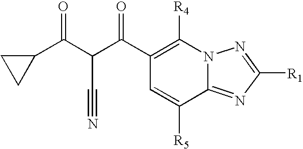 | |

| Cmpd No | R₁ | R₄ | R₅ | Phys. data |
|---|---|---|---|---|
| 36.1 | H | CH₃ | H | |
| 36.2 | Br | CH₃ | H | |
| 36.3 | CH₃ | CH₃ | H | |
| 36.4 | CH₂OCH₃ | CH₃ | H | |
| 36.5 | CH₂SMe | CH₃ | H | |
| 36.6 | N(CH₃)₂ | CH₃ | H | |
| 36.7 | OCH₃ | CH₃ | H | |
| 36.8 | OCHF₂ | CH₃ | H | |
| 36.9 | 4-Cl-Ph | CH₃ | H | |
| 36.10 | SCH₃ | CH₃ | H | |
| 36.11 | 3-pyridyl | CH₃ | H | |

TABLE 37
Compounds of formula I-5:

| | |
|---|---|
| | (I-5) |
| 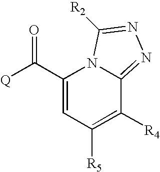 | |

| Cmpd No | Q | R₂ | R₄ | R₅ | Phys. Data |
|---|---|---|---|---|---|
| 37.1 | BIOD | H | CF₃ | H | |
| 37.2 | BIOD | CH₃ | CF₃ | H | |
| 37.3 | BIOD | H | CF₂H | H | |
| 37.4 | BIOD | CH₃ | CF₂H | H | |
| 37.5 | BIOD | H | CH₃ | H | |
| 37.6 | BIOD | CH₃ | CH₃ | H | |
| 37.7 | CHD | H | CF₃ | H | |
| 37.8 | CHD | CH₃ | CF₃ | H | |
| 37.9 | CHD | H | CF₂H | H | |
| 37.10 | CHD | CH₃ | CF₂H | H | |
| 37.11 | CHD | H | CH₃ | H | |
| 37.12 | CHD | CH₃ | CH₃ | H | |
| 37.13 | 5Me-CHD | H | CF₃ | H | |
| 37.14 | 5Me-CHD | CH₃ | CF₃ | H | ¹H-NMR (CDCl₃, ppm): 7.56(d, 1 H), 6.56(d, 1 H), |
| | | | | | 1.08(d, 3 H) |
| 37.15 | 5Me-CHD | H | CF₂H | H | |
| 37.16 | 5Me-CHD | CH₃ | CF₂H | H | |
| 37.17 | 5Me-CHD | H | CH₃ | H | |
| 37.18 | 5Me-CHD | CH₃ | CH₃ | H | |
| 37.19 | Sync | H | CF₃ | H | |
| 37.20 | Sync | CH₃ | CF₃ | H | |
| 37.21 | Sync | H | CF₂H | H | |
| 37.22 | Sync | CH₃ | CF₂H | H | |
| 37.23 | Sync | H | CH₃ | H | |
| 37.24 | Sync | CH₃ | CH₃ | H | |
| 37.25 | N-Me-py | H | CF₃ | H | |
| 37.26 | N-Me-py | CH₃ | CF₃ | H | |
| 37.27 | N-Me-py | H | CF₂H | H | |
| 37.28 | N-Me-py | CH₃ | CF₂H | H | |
| 37.29 | N-Me-py | H | CH₃ | H | |
| 37.30 | N-Me-py | CH₃ | CH₃ | H | |
| 37.31 | IFT | H | CF₃ | H | |
| 37.32 | IFT | CH₃ | CF₃ | H | |
| 37.33 | IFT | H | CF₂H | H | |
| 37.34 | IFT | CH₃ | CF₂H | H | |
| 37.35 | IFT | H | CH₃ | H | |
| 37.36 | IFT | CH₃ | CH₃ | H | |

TABLE 38
Compounds of formula I-6:

| | |
|---|---|
| | (I-6) |
| 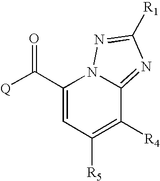 | |

| Cmpd No | Q | R₁ | R₄ | R₅ | Phys. Data |
|---|---|---|---|---|---|
| 38.1 | BIOD | H | CF₃ | H | |
| 38.2 | BIOD | CH₃ | CF₃ | H | |
| 38.3 | BIOD | H | CF₂H | H | |
| 38.4 | BIOD | CH₃ | CF₂H | H | |
| 38.5 | BIOD | H | CH₃ | H | |
| 38.6 | BIOD | CH₃ | CH₃ | H | |
| 38.7 | CHD | H | CF₃ | H | |
| 38.8 | CHD | CH₃ | CF₃ | H | |
| 38.9 | CHD | H | CF₂H | H | |
| 38.10 | CHD | CH₃ | CF₂H | H | |
| 38.11 | CHD | H | CH₃ | H | |
| 38.12 | CHD | CH₃ | CH₃ | H | |
| 38.13 | 5Me-CHD | H | CF₃ | H | |
| 38.14 | 5Me-CHD | CH₃ | CF₃ | H | |
| 38.15 | 5Me-CHD | H | CF₂H | H | |
| 38.16 | 5Me-CHD | CH₃ | CF₂H | H | |
| 38.17 | 5Me-CHD | H | CH₃ | H | |
| 38.18 | 5Me-CHD | CH₃ | CH₃ | H | |
| 38.19 | Sync | H | CF₃ | H | |
| 38.20 | Sync | CH₃ | CF₃ | H | |
| 38.21 | Sync | H | CF₂H | H | |
| 38.22 | Sync | CH₃ | CF₂H | H | |
| 38.23 | Sync | H | CH₃ | H | |
| 38.24 | Sync | CH₃ | CH₃ | H | |
| 38.25 | N-Me-py | H | CF₃ | H | |
| 38.26 | N-Me-py | CH₃ | CF₃ | H | |
| 38.27 | N-Me-py | H | CF₂H | H | |
| 38.28 | N-Me-py | CH₃ | CF₂H | H | |
| 38.29 | N-Me-py | H | CH₃ | H | |
| 38.30 | N-Me-py | CH₃ | CH₃ | H | |
| 38.31 | IFT | H | CF₃ | H | |
| 38.32 | IFT | CH₃ | CF₃ | H | |
| 38.33 | IFT | H | CF₂H | H | |
| 38.34 | IFT | CH₃ | CF₂H | H | |
| 38.35 | IFT | H | CH₃ | H | |
| 38.36 | IFT | CH₃ | CH₃ | H | |

TABLE 39
Compounds of formula I-7:

| | |
|---|---|
| | (I-7) |
| 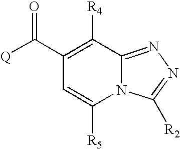 | |

| Cmpd No | Q | R₂ | R₄ | R₅ | Phys. Data |
|---|---|---|---|---|---|
| 39.1 | BIOD | H | CF₃ | H | |
| 39.2 | BIOD | CH₃ | CF₃ | H | |
| 39.3 | BIOD | H | CF₂H | H | |
| 39.4 | BIOD | CH₃ | CF₂H | H | |
| 39.5 | BIOD | H | CH₃ | H | |
| 39.6 | BIOD | CH₃ | CH₃ | H | |
| 39.7 | CHD | H | CF₃ | H | |
| 39.8 | CHD | CH₃ | CF₃ | H | |
| 39.9 | CHD | H | CF₂H | H | |
| 39.10 | CHD | CH₃ | CF₂H | H | |
| 39.11 | CHD | H | CH₃ | H | |
| 39.12 | CHD | CH₃ | CH₃ | H | |
| 39.13 | 5Me-CHD | H | CF₃ | H | |
| 39.14 | 5Me-CHD | CH₃ | CF₃ | H | |
| 39.15 | 5Me-CHD | H | CF₂H | H | |
| 39.16 | 5Me-CHD | CH₃ | CF₂H | H | |
| 39.17 | 5Me-CHD | H | CH₃ | H | |
| 39.18 | 5Me-CHD | CH₃ | CH₃ | H | |
| 39.19 | Sync | H | CF₃ | H | |
| 39.20 | Sync | CH₃ | CF₃ | H | |
| 39.21 | Sync | H | CF₂H | H | |
| 39.22 | Sync | CH₃ | CF₂H | H | |
| 39.23 | Sync | H | CH₃ | H | |
| 39.24 | Sync | CH₃ | CH₃ | H | |
| 39.25 | N-Me-py | H | CF₃ | H | |
| 39.26 | N-Me-py | CH₃ | CF₃ | H | |
| 39.27 | N-Me-py | H | CF₂H | H | |
| 39.28 | N-Me-py | CH₃ | CF₂H | H | |
| 39.29 | N-Me-py | H | CH₃ | H | |
| 39.30 | N-Me-py | CH₃ | CH₃ | H | |
| 39.31 | IFT | H | CF₃ | H | |
| 39.32 | IFT | CH₃ | CF₃ | H | |
| 39.33 | IFT | H | CF₂H | H | |
| 39.34 | IFT | CH₃ | CF₂H | H | |
| 39.35 | IFT | H | CH₃ | H | |
| 39.36 | IFT | CH₃ | CH₃ | H | |

TABLE 40
Compounds of formula I-8:

| | |
|---|---|
| | (I-8) |
| 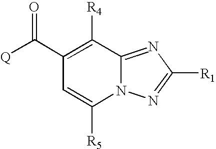 | |

| Cmpd No | Q | R₁ | R₄ | R₅ | Phys. Data |
|---|---|---|---|---|---|
| 40.1 | BIOD | H | CF₃ | H | |
| 40.2 | BIOD | CH₃ | CF₃ | H | |
| 40.3 | BIOD | H | CF₂H | H | |
| 40.4 | BIOD | CH₃ | CF₂H | H | |
| 40.5 | BIOD | H | CH₃ | H | |
| 40.6 | BIOD | CH₃ | CH₃ | H | |
| 40.7 | CHD | H | CF₃ | H | |
| 40.8 | CHD | CH₃ | CF₃ | H | |
| 40.9 | CHD | H | CF₂H | H | |
| 40.10 | CHD | CH₃ | CF₂H | H | |
| 40.11 | CHD | H | CH₃ | H | |
| 40.12 | CHD | CH₃ | CH₃ | H | |
| 40.13 | 5Me-CHD | H | CF₃ | H | |
| 40.14 | 5Me-CHD | CH₃ | CF₃ | H | |
| 40.15 | 5Me-CHD | H | CF₂H | H | |
| 40.16 | 5Me-CHD | CH₃ | CF₂H | H | |
| 40.17 | 5Me-CHD | H | CH₃ | H | |
| 40.18 | 5Me-CHD | CH₃ | CH₃ | H | |
| 40.19 | Sync | H | CF₃ | H | |
| 40.20 | Sync | CH₃ | CF₃ | H | |
| 40.21 | Sync | H | CF₂H | H | |
| 40.22 | Sync | CH₃ | CF₂H | H | |
| 40.23 | Sync | H | CH₃ | H | |
| 40.24 | Sync | CH₃ | CH₃ | H | |
| 40.25 | N-Me-py | H | CF₃ | H | |
| 40.26 | N-Me-py | CH₃ | CF₃ | H | |
| 40.27 | N-Me-py | H | CF₂H | H | |
| 40.28 | N-Me-py | CH₃ | CF₂H | H | |
| 40.29 | N-Me-py | H | CH₃ | H | |
| 40.30 | N-Me-py | CH₃ | CH₃ | H | |
| 40.31 | IFT | H | CF₃ | H | |
| 40.32 | IFT | CH₃ | CF₃ | H | |
| 40.33 | IFT | H | CF₂H | H | |
| 40.34 | IFT | CH₃ | CF₂H | H | |
| 40.35 | IFT | H | CH₃ | H | |
| 40.36 | IFT | CH₃ | CH₃ | H | |

TABLE 41
Compounds of formula I-1j:

| | |
|---|---|
| | (I-1j) |
| 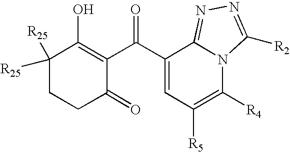 | |

| Cmpd No | R₂₄ | R₂₅ | R₂ | R₄ | R₅ | Phys. data |
|---|---|---|---|---|---|---|
| 41.1 | —CH₂CH₂— | | H | CF₃ | H | |
| 41.2 | —CH₂CH₂— | | CH₃ | CF₃ | H | 1.27 min; 366, |
| | | | | | | 364 |
| 41.3 | —CH₂CH₂— | | CH₂OCH₃ | CF₃ | H | |
| 41.4 | —CH₂CH₂— | | OCH₃ | CF₃ | H | |
| 41.5 | n-propyl | H | H | CF₃ | H | |
| 41.6 | n-propyl | H | CH₃ | CF₃ | H | 1.48 min; 382, |
| | | | | | | 380 |
| 41.7 | n-propyl | H | CH₂OCH₃ | CF₃ | H | |
| 41.8 | n-propyl | H | OCH₃ | CF₃ | H | |

TABLE 42
Compounds of formula I-1k

| | |
|---|---|
| | (I-1k) |
| 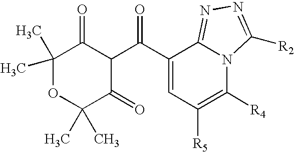 | |

| Cmpd No | R₂ | R₄ | R₅ | Physchem data |
|---|---|---|---|---|
| 42.1 | H | CF₃ | H | |
| 42.2 | Cl | CF₃ | H | 1.21 min; 420, 418 |
| 42.3 | Br | CF₃ | H | |
| 42.4 | CH₃ | CF₃ | H | |
| 42.5 | CH(CH₃)₂ | CF₃ | H | |
| 42.6 | CH₂OCH₃ | CF₃ | H | |
| 42.7 | CH₂SMe | CF₃ | H | |
| 42.8 | N(CH₃)₂ | CF₃ | H | |
| 42.9 | OCH₃ | CF₃ | H | |
| 42.1 | OCHF₂ | CF₃ | H | |
| 42.11 | 4-Cl-Ph | CF₃ | H | |
| 42.12 | SCH₃ | CF₃ | H | |
| 42.13 | 3-pyridyl | CF₃ | H | |

TABLE 43
Compounds of formula I-1I

| | |
|---|---|
| | (I-1I) |
| 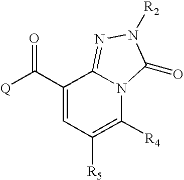 | |

| Cmpd No | Q | R₂ | R₄ | R₅ | Physchem data |
|---|---|---|---|---|---|
| 43.1 | BIOD | H | CF₃ | H | |
| 43.2 | BIOD | CH₃ | CF₃ | H | 1.24 min; 382, 380 |
| 43.3 | BIOD | Et | CF₃ | H | 1.36 min, 396, 394 |
| 43.4 | BIOD | Bz | CF₃ | H | 1.64 min, 458 |
| 43.5 | BIOD | H | Cl | H | |
| 43.6 | BIOD | CH₃ | Cl | H | |
| 43.7 | BIOD | H | CH₃ | H | |
| 43.8 | BIOD | CH₃ | CH₃ | H | |
| 43.9 | CHD | H | CF₃ | H | |
| 43.10 | CHD | CH₃ | CF₃ | H | |
| 43.11 | CHD | Et | CF₃ | H | |
| 43.12 | CHD | Bz | CF₃ | H | 1.52 min, 430 |
| 43.13 | CHD | H | Cl | H | |
| 43.14 | CHD | CH₃ | Cl | H | |
| 43.15 | CHD | H | CH₃ | H | |
| 43.16 | CHD | CH₃ | CH₃ | H | |
| 43.17 | 5Me- | H | CF₃ | H | |
| | CHD | | | | |
| 43.18 | 5Me- | CH₃ | CF₃ | H | |
| | CHD | | | | |
| 43.19 | 5Me- | H | Cl | H | |
| | CHD | | | | |
| 43.20 | 5Me- | CH₃ | Cl | H | |
| | CHD | | | | |
| 43.21 | 5Me- | H | CH₃ | H | |
| | CHD | | | | |
| 43.22 | 5Me- | CH₃ | CH₃ | H | |
| | CHD | | | | |
| 43.23 | Sync | H | CF₃ | H | |
| 43.24 | Sync | CH₃ | CF₃ | H | |
| 43.25 | Sync | H | Cl | H | |
| 43.26 | Sync | CH₃ | Cl | H | |
| 43.27 | Sync | H | CH₃ | H | |
| 43.28 | Sync | CH₃ | CH₃ | H | |
| 43.29 | N-Me-py | H | CF₃ | H | |
| 43.30 | N-Me-py | CH₃ | CF₃ | H | |
| 43.31 | N-Me-py | H | Cl | H | |
| 43.32 | N-Me-py | CH₃ | Cl | H | |
| 43.33 | N-Me-py | H | CH₃ | H | |
| 43.34 | N-Me-py | CH₃ | CH₃ | H | |
| 43.35 | IFT | H | CF₃ | H | |
| 43.36 | IFT | CH₃ | CF₃ | H | |
| 43.37 | IFT | H | Cl | H | |
| 43.38 | IFT | CH₃ | Cl | H | |
| 43.39 | IFT | H | CH₃ | H | |
| 43.40 | IFT | CH₃ | CH₃ | H | |

The following Table lists preferred compounds of formula II.

TABLE 44
Compounds of formula IIa:

| | |
|---|---|
| | (IIA) |
| 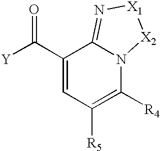 | |

| Cmpd No | X₁ | X₂ | R₄ | R₅ | Y | Phys. data |
|---|---|---|---|---|---|---|
| Z1.001 | N | C—CH₃ | CF₃ | H | OCH₂CH₃ | m.p.: 151-152° C. |
| Z1.002 | N | C—CH₃ | CF₃ | H | OH | m.p.: 212-213° C. |
| Z1.003 | N | C—CH₃ | CF₃ | H | p-NO₂-phenoxy | m.p.: >220° C. |
| Z1.004 | C—CH₃ | N | CF₃ | H | OCH₂CH₃ | m.p.: 86-87° C. |
| Z1.005 | C—CH₃ | N | CF₃ | H | OH | m.p.: 150-151° C. |
| Z1.006 | C—CH₃ | N | CF₃ | H | p-NO₂-phenoxy | m.p.: >220° C. |
| Z1.007 | N | C—CH₂Cl | CF₃ | H | OCH₂CH₃ | m.p.: 143-144° C. |
| Z1.008 | N | C—CH₂Cl | CF₃ | H | OH | ¹H-NMR (CDCl₃, ppm): |
| | | | | | | 8.18(d, 1 H), 7.66(d, |
| | | | | | | 1 H), 5.28(s, 2 H) |
| Z1.009 | N | C—CH₂CH₃ | CF₃ | H | OCH₂CH₃ | m.p.: 120-121° C. |
| Z1.010 | N | C—CH₂CH₃ | CF₃ | H | OH | m.p.: 150-151° C. |

### BIOLOGICAL EXAMPLES

### Example B1

### Herbicidal Action Prior to Emergence of the Plants (Pre-Emergence Action)

Monocotyledonous and dicotyledonous test plants are sown in seed trays in standard soil. Immediately after sowing, the test compounds are applied by spraying in the form of an aqueous suspension (prepared from a wettable powder (Example F3, b) according to WO 97/34485) or in the form of an emulsion (prepared from an emulsifiable concentrate (Example F1, c) according to WO 97/34485) in a concentration of 250 g/ha. The test plants are then grown in a greenhouse under optimum conditions. After a 4-week test period, the test is evaluated (100%=total damage to plant; 0%=no damage to plant; NC not evaluated).

TABLE B1
preemergence application:

| Comp No | g/ha | SETFA | DIGSA | ECHCG | BRAPL | ABUTH | AMARE | STEME | VERPE |
|---|---|---|---|---|---|---|---|---|---|
| 3.3 | 250 | 0 | 0 | 0 | 0 | 100 | 100 | 70 | 30 |
| 6.3 | 250 | 0 | 30 | 30 | NC | 10 | 70 | 40 | 80 |
| 3.6 | 250 | 50 | 90 | 90 | 60 | 90 | 70 | 20 | 20 |
| 1.8 | 250 | 60 | 100 | 40 | 100 | 100 | 100 | NC | 70 |
| 5.4 | 250 | 30 | 60 | 40 | 100 | 100 | 100 | 100 | 100 |
| 3.5 | 250 | 90 | 90 | 70 | 80 | 100 | 70 | 60 | 30 |
| 1.7 | 250 | 80 | 100 | 80 | 80 | 100 | 100 | 100 | 100 |
| 1.5 | 250 | 80 | 100 | 60 | 60 | 100 | 100 | 100 | 70 |
| 5.10 | 250 | 0 | 40 | 20 | 20 | 100 | 100 | 30 | NC |
| 1.16 | 250 | 80 | 80 | 100 | 70 | 100 | 100 | 100 | 50 |
| 1.4 | 250 | 90 | 100 | 90 | 70 | 100 | 100 | 100 | 100 |
| 1.54 | 250 | 0 | 0 | 0 | 40 | 20 | 10 | 0 | 20 |
| 13.3 | 250 | 70 | 80 | 30 | 80 | 100 | 90 | 100 | 100 |
| 1.1 | 250 | 30 | 70 | 0 | 30 | 100 | NC | 100 | 60 |
| 3.7 | 250 | 10 | 30 | 10 | 30 | 30 | 30 | 20 | 10 |
| 2.4 | 250 | 20 | 30 | NC | 10 | 70 | 60 | 60 | 10 |
| 2.7 | 250 | 0 | 0 | 10 | 0 | 30 | 20 | 20 | 50 |
| 5.5 | 250 | 20 | 10 | 60 | 20 | NC | 60 | 70 | 90 |
| 4.3 | 250 | 40 | 100 | 100 | 100 | 70 | 100 | 80 | 100 |
| 41.2 | 250 | 80 | 100 | 100 | 80 | 80 | 100 | 70 | 90 |
| 41.6 | 250 | 70 | 90 | 70 | 60 | 70 | 100 | 50 | 50 |
| 2.2 | 250 | 10 | 50 | 70 | 50 | 50 | 70 | 0 | 60 |
| 3.2 | 250 | 0 | 80 | 70 | 0 | 100 | 100 | 100 | 100 |
| 1.2 | 250 | 90 | 100 | 100 | 100 | 100 | 100 | NC | 70 |
| 1.212 | 250 | 80 | 70 | 50 | 60 | 70 | 100 | 70 | 0 |
| 2.209 | 250 | 80 | 80 | 70 | 90 | 100 | 100 | 70 | 30 |
| 1.211 | 250 | 30 | 70 | 70 | 100 | 30 | 100 | 80 | 90 |
| 2.35 | 250 | 0 | 20 | 0 | 50 | 0 | 0 | 50 | 0 |
| 2.1 | 250 | 90 | 100 | 90 | 100 | 100 | 100 | 70 | 40 |
| 3.1 | 250 | 0 | 100 | 10 | 40 | 100 | 100 | 70 | 60 |
| 2.4 | 250 | 20 | 30 | NC | 10 | 70 | 60 | 60 | 10 |
| 5.1 | 250 | 40 | 100 | 90 | 100 | 100 | 100 | 100 | 80 |
| 3.15 | 250 | 30 | 30 | 30 | 30 | 10 | 0 | 60 | 30 |
| 11.3 | 250 | 10 | 30 | 0 | 50 | 70 | 40 | 40 | 40 |
| 2.7 | 250 | 0 | 0 | 10 | 0 | 30 | 20 | 20 | 50 |
| 1.16 | 250 | 80 | 80 | 100 | 70 | 100 | 100 | 100 | 50 |
| 5.5 | 250 | 20 | 10 | 60 | 20 | NC | 60 | 70 | 90 |
| 3.7 | 250 | 10 | 30 | 10 | 30 | 30 | 30 | 20 | 10 |
| 1.35 | 250 | 30 | 100 | 0 | 100 | 50 | 100 | 70 | 40 |
| 3.12 | 250 | 0 | 10 | 0 | 60 | 50 | 100 | 0 | NC |
| 5.10 | 250 | 0 | 40 | 20 | 20 | 100 | 100 | 30 | NC |
| 1.5 | 250 | 80 | 100 | 60 | 60 | 100 | 100 | 100 | 70 |
| 3.4 | 250 | 30 | 70 | 70 | 80 | 80 | 60 | 30 | 50 |
| 1.7 | 250 | 80 | 100 | 80 | 80 | 100 | 100 | 100 | 100 |
| 1.8 | 250 | 60 | 100 | 40 | 100 | 100 | 100 | NC | 70 |
| 3.6 | 250 | 50 | 90 | 90 | 60 | 90 | 70 | 20 | 20 |
| 5.3 | 250 | 60 | 100 | 100 | 70 | 100 | 100 | 100 | 100 |
| 4.3 | 250 | 40 | 100 | 100 | 100 | 70 | 100 | 80 | 100 |
| 6.3 | 250 | 0 | 30 | 30 | NC | 10 | 70 | 40 | 80 |

### Example B2

### Herbicidal Action After Emergence of the Plants (Post-Emergence Herbicidal Action)

Monocotyledonous and dicotyledonous test plants are sown in seed trays in standard soil. When the test plants are at the 2- to 3-leaf stage, the test compounds are applied by spraying in the form of an aqueous suspension (prepared from a wettable powder (Example F3, b) according to WO 97/34485) or in the form of an emulsion (prepared from an emulsifiable concentrate (Example F1, c) according to WO 97/34485) in a concentration of 125 g/ha. The test plants are then grown on in a greenhouse under optimum conditions. After a 2- to 3-week test period, the test is evaluated (100%=total damage to plant; 0%=no damage to plant).

TABLE B2
postemergent application:

| Comp No | g/ha | SETFA | DIGSA | ABUTH | XANST | AMARE | CHEAL | SINAR | VERPE |
|---|---|---|---|---|---|---|---|---|---|
| 3.3 | 125 | 30 | 70 | 70 | 80 | 70 | 80 | 70 | 80 |
| 6.3 | 125 | 60 | 80 | 60 | 80 | 90 | 70 | 80 | 80 |
| 3.6 | 125 | 70 | 70 | 80 | 90 | 70 | 90 | 90 | 100 |
| 1.8 | 125 | 80 | 80 | 90 | 90 | 90 | 90 | 90 | 100 |
| 5.4 | 125 | 50 | 50 | 80 | 90 | 70 | 80 | 80 | 90 |
| 3.5 | 125 | 70 | 80 | 90 | 80 | 100 | 90 | 80 | 90 |
| 1.7 | 125 | 70 | 80 | 90 | 80 | 90 | 80 | 80 | 90 |
| 1.5 | 125 | 60 | 70 | 90 | 80 | 70 | 80 | 80 | 70 |
| 5.10 | 125 | 30 | 20 | 80 | 80 | 80 | 70 | 80 | 100 |
| 1.16 | 125 | 70 | 70 | 80 | 80 | 90 | 80 | 80 | 80 |
| 1.4 | 125 | 90 | 80 | 70 | 70 | 70 | 90 | 80 | 80 |
| 1.54 | 125 | 50 | 60 | 70 | 70 | 80 | 70 | 60 | 60 |
| 13.3 | 125 | 70 | 60 | 80 | 80 | 70 | 50 | 80 | 70 |
| 1.1 | 125 | 80 | 60 | 80 | 80 | 80 | 80 | 90 | 50 |
| 3.7 | 125 | 80 | 80 | 80 | 80 | 90 | 70 | 90 | 80 |
| 2.4 | 125 | 70 | 70 | 80 | 80 | 80 | 80 | 80 | 30 |
| 2.7 | 125 | 20 | 20 | 70 | 70 | NC | 70 | 60 | 10 |
| 5.5 | 125 | 50 | 10 | 80 | 70 | 90 | 80 | 70 | 80 |
| 41.6 | 125 | 70 | 80 | 80 | 80 | 80 | 80 | 80 | 80 |
| 2.2 | 125 | 80 | 90 | 40 | 60 | 80 | 80 | 70 | 70 |
| 1.2 | 125 | 90 | 90 | 90 | 80 | 80 | 80 | 70 | 70 |
| 1.212 | 125 | 80 | 80 | 80 | 80 | 80 | 80 | 80 | 80 |
| 2.209 | 125 | 80 | 80 | 80 | 80 | 90 | 80 | 80 | 80 |
| 1.211 | 125 | 80 | 70 | 70 | 80 | 100 | 80 | 80 | 80 |
| 1.16 | 125 | 70 | 70 | 80 | 80 | 90 | 80 | 80 | 80 |
| 5.4 | 125 | 50 | 10 | 80 | 70 | 90 | 80 | 70 | 80 |
| 3.7 | 125 | 80 | 80 | 80 | 80 | 90 | 70 | 90 | 80 |
| 3.4 | 125 | 80 | 80 | 90 | 80 | 80 | 60 | 90 | 100 |
| 5.3 | 125 | 50 | 50 | 80 | 90 | 70 | 80 | 80 | 90 |
| 1.8 | 125 | 80 | 80 | 90 | 90 | 90 | 90 | 90 | 100 |
| 5.2 | 125 | 80 | 70 | 90 | 70 | 80 | 100 | 90 | 90 |
| 6.3 | 125 | 60 | 80 | 60 | 80 | 90 | 70 | 80 | 80 |

### Example B3

### Microscreen (Pre-Emergence)

Monocotyledonous and dicotyledonous test plants are sown in sterilised standard soil in seed trays each having 96 cells. After being cultivated for about 9 days under controlled conditions in a climatic chamber (cultivation at 17/23° C.; 13 hours light; 50-60% humidity; after the application at 19/24° C.), the plants Nasturtium officinale, Agrostis tenuis, Stellaria media, Digitaria sanguinalis, Solanum nigrum, Amaranthus retroflexus, Setaria italica, are treated with an aqueous spray solution of 1 g/l, 0.25 g/l or 0.063 g/l of the active ingredient being used (amount applied: 500 g/l; incl. 10% DMSO as solvent). The plants are grown on in the climatic chamber until, 8 days later, the test is evaluated (100%=total damage to plant; 0%=no damage to plant).

TABLE B3
preemergent application, microscreen:

| Ex. Nr. | g/ha | Digitaria | Agrostis | Setaria | Stellaria | Nasturtium | Amaranthus | Solanum |
|---|---|---|---|---|---|---|---|---|
| 1.4 | 250 | 90 | 50 | 70 | 80 | 90 | 90 | 90 |
| 1.8 | 250 | 100 | 100 | 100 | 80 | 100 | 0 | 90 |
| 41.2 | 250 | 100 | 100 | 80 | 90 | 100 | 100 | 100 |
| 41.6 | 250 | 90 | 100 | 0 | 80 | 100 | 100 | 90 |
| 4.3 | 250 | 100 | 100 | 60 | 80 | 100 | 100 | 100 |
| 5.3 | 250 | 100 | 50 | 50 | 90 | 100 | 100 | 100 |
| 3.4 | 250 | 100 | 100 | 60 | 70 | 80 | 80 | 0 |
| 1.5 | 250 | 80 | 100 | 50 | 60 | 80 | 30 | 100 |

What is claimed is:

1. A compound of formula I 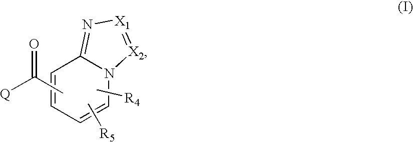
wherein
X₁ is nitrogen, if X₂ is CR₂; or CR₁, if X₂ is nitrogen; or NR₅₁, if X₂ is C(O); or C(O), if X₂ is NR₅₂;
X₂ is nitrogen, if X₁ is CR₁; or CR₂, if X₁ is nitrogen; or NR₅₂, if X₁ is C(O); or C(O), if X₁ is NR₅₁;
R₅₁ and R₅₂ independently from each other, are hydrogen, a group —X₆ or a group —X₄—X₅—X₆;
R₁ and R₂ independently from each other, are hydrogen, halogen, hydroxy, mercapto, amino, azido, SF₅, nitro, cyano, rhodano, carbamoyl, carboxy, formyl, tri(C₁-C₄alkyl)silyl, C₁-C₄alkyl(C₁-C₄alkoxy)phosphino or di(C₁-C₄alkoxy)phosphono;
or R₁ and R₂ independently from each other are a group —X₆, a group —X₅—X₆ or a group —X₄—X₅—X₆, wherein
X₄ is C₁-C₆alkylene, C₂-C₆alkenylene or C₂-C₆alkynylene, which can be mono- or poly-substituted by halogen, hydroxy, C₁-C₆alkoxy, C₃-C₆cycloalkyloxy, C₁-C₆alkoxy-C₁-C₆alkoxy, C₁-C₆alkoxy-C₁-C₆alkoxy-C₁-C₆alkoxy or C₁-C₂alkylsulfonyloxy; or by a bivalent C₁-C₈ alkylene group which may be interrupted by 1 to 2 oxygen atoms, sulphur or NRa₂₆, said bivalent C₁-C₈ alkylene group can be substituted by substituents from the group consisting of halogen, hydroxy, mercapto, amino, formyl, carboxy, nitro, cyano, carbamoyl, C₁-C₆alkoxy, C₁-C₆alkoxycarbonyl, C₁-C₆-alkylaminocarbonyl, C₁-C₆-dialkylaminocarbonyl, C₂-C₆alkenyl, C₂-C₆haloalkenyl, C₂-C₆alkynyl, C₂-C₆haloalkynyl, C₂-C₆alkenyloxy, C₂-C₆alkynyloxy, C₁-C₆haloalkoxy, C₂-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆alkoxy, formyloxy, C₁-C₆alkylcarbonyloxy, C₁-C₆alkylcarbonyl, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, C₁-C₆alkylthiocarbonyl, C₁-C₆alkylamino, di(C₁-C₆alkyl)amino, C₁-C₄alkylsulfonyloxy, C₁-C₄alkylcarbonylamino, N(C₁-C₄alkyl)-C₁-C₄alkylcarbonylamino, C₁-C₄alkoxycarbonylamino, N(C₁-C₄alkyl)-C₁-C₄alkoxycarbonylamino, C₁-C₄alkylsulfonylamino, N(C₁-C₄alkyl)-C₁-C₄alkylsulfonylamino, OSO₂—C₁-C₄-alkyl, rhodano, tri(C₁-C₄alkyl)silyl, C₁-C₄alkyl(C₁-C₄alkoxy)phosphino and di(C₁-C₄alkoxy)phosphono;
X₅ is oxygen, —OC(O)—, —OC(O)O—, —OC(O)N(R₃)—, —ON(Ra₂₁)—, —ON═C(Ra₂₂)—, OS(O)₂—, OS(O)₂O—, OS(O)₂N(R₃)—, thio, sulfinyl, sulfonyl, —SO₂N(R₃)—, —S(O)₂O—, —S(═NRa₂₃)(O)—, —C(O)O—, —C(O)—, —C(O)N(R₃)—, —C(Ra₂₂)═NO—, —N(Ra₂₁)O—, —N(R₃)SO₂—, —N(Ra₂₄)—, —N(R₃)C(O)—, —N(R₃)C(O)O—, —N(R₃)C(O)N(R₃)—, —N(R₃)SO₂O—, —N(R₃)SO₂N(R₃)—, —N═S(Ra₂₅)(O)— or —S(Ra₂₅)(O)═N—;
X₆ is C₁-C₆alkyl, C₂-C₆alkenyl or C₂-C₆alkynyl; or C₁-C₆alkyl, C₂-C₆alkenyl or C₂-C₆alkynyl mono- or poly-substituted by halogen, hydroxy, mercapto, amino, formyl, carboxy, nitro, cyano, carbamoyl, C₁-C₆alkoxy, C₁-C₆alkoxycarbonyl, C₁-C₆-alkylaminocarbonyl, C₁-C₆-dialkylaminocarbonyl, C₂-C₆alkenyl, C₂-C₆haloalkenyl, C₂-C₆alkynyl, C₂-C₆haloalkynyl, C₂-C₆alkenyloxy, C₂-C₆alkynyloxy, C₁-C₆haloalkoxy, C₂-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆-alkoxy, formyloxy, C₁-C₆alkylcarbonyloxy, C₁-C₆alkylcarbonyl, C₁-C₆alkylthio, C₁-C₆alkylsulfinyl, C₁-C₆alkylsulfonyl, C₁-C₆haloalkylthio, C₁-C₆haloalkylsulfinyl or C₁-C₆halo-alkylsulfonyl, C₁-C₆alkylthiocarbonyl, C₁-C₆alkylamino, di(C₁-C₆alkyl)amino, C₁-C₄alkylsulfonyloxy, C₁-C₄alkylcarbonylamino, N(C₁-C₄alkyl)-C₁-C₄alkylcarbonylamino, C₁-C₄alkoxycarbonylamino, N(C₁-C₄alkyl)-C₁-C₄alkoxycarbonylamino, C₁-C₄alkylsulfonylamino, N(C₁-C₄alkyl)-C₁-C₄alkylsulfonylamino, OSO₂—C₁-C₄-alkyl, rhodano, tri(C₁-C₄alkyl)silyl, C₁-C₄alkyl(C₁-C₄alkoxy)phosphino or di(C₁-C₄alkoxy)phosphono;
or X₆ is a three- to ten-membered mono- or bicyclic ring system, which may be aromatic, saturated or partially saturated and can contain from 1 to 4 hetero atoms selected from the group consisting of nitrogen, oxygen, sulfur, —S(O)—, —S(O)₂—, —N(Ra₂₆)—, —C(O)— and C(═NORa₇), and each ring system can contain not more than two oxygen atoms and not more than two sulfur atoms, and the ring system can itself be mono- or poly-substituted by C₁-C₆alkyl, C₁-C₆haloalkyl, C₂-C₆alkenyl, C₂-C₆haloalkenyl, C₂-C₆alkynyl, C₂-C₆haloalkynyl, hydroxy, C₁-C₆alkoxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₄alkylaminosulfonyl, di(C₁-C₄alkyl)aminosulfonyl, amino, C₁-C₄alkylamino, di(C₁-C₄alkyl)amino, halogen, cyano, nitro, phenyl, phenoxy, phenylthio, benzyloxy and/or by benzylthio, it being possible for phenyl groups in turn to be substituted on the phenyl ring by C₁-C₃alkyl, C₁-C₃haloalkyl, C₁-C₃alkoxy, C₁-C₃haloalkoxy, C₁-C₃alkylsulfonyl C₁-C₃haloalkyl-sulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;
R₃ is hydrogen, C₁-C₄alkyl, C₁-C₄haloalkyl, C₁-C₂alkoxy-C₁-C₂alkyl or phenyl, which in turn can be mono- or poly-substituted by C₁-C₃alkyl, C₁-C₃haloalkyl, C₁-C₃alkoxy, C₁-C₃haloalkoxy, C₁-C₃alkylsulfonyl C₁-C₃haloalkylsulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;
Ra₂₁ is hydrogen, C₁-C₄alkyl or C₁-C₂alkoxy-C₁-C₂alkyl;
Ra₂₂ is hydrogen, C₁-C₄alkyl or phenyl, which may be mono- or poly-substituted by C₁-C₃alkyl, C₁-C₃haloalkyl, C₁-C₃alkoxy, C₁-C₃haloalkoxy, C₁-C₃alkylsulfonyl C₁-C₃haloalkyl-sulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;
Ra₂₃ is hydrogen, formyl, C₁-C₄alkyl, C₁-C₄alkylcarbonyl, C₁-C₄haloalkylcarbonyl or C₁-C₄ alkoxycarbonyl;
Ra₂₅ is C₁-C₄alkyl, or is benzyl which can be mono- or polysubstituted by C₁-C₃alkyl, C₁-C₃haloalkyl, C₁-C₃alkoxy, C₁-C₃haloalkoxy, C₁-C₃alkylsulfonyl C₁-C₃haloalkylsulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro;
Ra₂₄ and Ra₂₆ independently from each other, are hydrogen, C₁-C₄alkyl, C₁-C₄alkylthiocarbonyl, C₁-C₄alkoxycarbonyl, C₁-C₄alkylcarbonyl, C₃-C₄cycloalkylcarbonyl, phenylcarbonyl or phenyl, it being possible for the phenyl groups in turn to be mono- or polysubstituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, C₁-C₄alkylamino, di-C₁-C₄alkylamino, C₁-C₄alkyl-S, C₁-C₄alkyl-S(O), C₁-C₄alkyl-SO₂, C₁-C₄alkyl-S(O)₂O, C₁-C₄haloalkyl-S, C₁-C₄haloalkyl-S(O), C₁-C₄haloalkyl-SO₂, C₁-C₄haloalkyl-S(O)₂O, C₁-C₄alkyl-S(O)₂NH, C₁-C₄alkyl-S(O)₂N(C₁-C₄alkyl), halogen, nitro or by cyano;
R₄ is hydrogen, hydroxy, halogen, cyano, C₁-C₆-alkyl, C₁-C₆-haloalkyl, C₂-C₆-alkenyl, C₂-C₆-haloalkenyl, C₂-C₆-alkinyl, C₂-C₆-haloalkinyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆-haloalkylsulfinyl, C₁-C₆-haloalkylsulfonyl, C₁-C₆alkylaminosulfonyl, di-C₂-C₆alkylaminosulfonyl, C₁-C₆-alkylsulfonyloxy, amino, C₁-C₄alkylsulfonylamino, N(C₁-C₄alkyl)-C₁-C₄alkylsulfonylamino, nitro, triazolyl, furyl or phenyl, it being possible for phenyl in turn to be mono- or polysubstituted by C₁-C₃alkyl, C₁-C₃haloalkyl, C₁-C₃alkoxy, C₁-C₃haloalkoxy, C₁-C₃-alkylsulfonyl C₁-C₃haloalkylsulfonyl, aminosulfonyl, C₁-C₂alkylaminosulfonyl, di(C₁-C₂alkyl)-aminosulfonyl, di(C₁-C₄alkyl)amino, C₁-C₄alkoxycarbonyl, halogen, cyano or nitro;
R₅ is hydrogen, halogen, C₁-C₃alkyl, C₁-C₃haloalkyl or C₁-C₃alkoxy; or if R₅ is bound to the meta-position with regard to the carbonyl group and is hydrogen, the ortho-position with regard to the carbonyl group can be additionally cyano;
Q is a group Q₁ 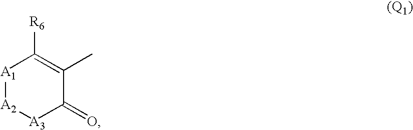
wherein
A₁ is C(R₁₁R₁₂) or NR₁₃;
A₂ is C(R₁₄R₁₅)ₘ, C(O), oxygen, NR₁₆ or S(O)_{q};
A₃ is C(R₁₇R₁₈) or NR₁₉;
with the proviso that A₂ is other than S(O)_{q} when A₁ is NR₁₃ and/or A₃ is NR₁₉;
R₆ is hydroxy, O⁻M⁺, wherein M⁺ is a metal cation or an ammonium cation; halogen or S(O)ₙR₉, wherein
m is 1 or 2;
q, n and k are each independently of the others 0, 1 or 2;
R₉ is C₁-C₁₂alkyl, C₂-C₁₂alkenyl, C₂-C₁₂alkynyl, C₃-C₁₂allenyl, C₃-C₁₂cycloalkyl, C₅-C₁₂cyclo-alkenyl, R₁₀-C₁-C₁₂alkylene or R₁₀-C₂-C₁₂alkenylene, wherein alkylene or alkenylene may be interrupted by —O—, —S(O)ₖ— and/or —C(O)— and can be mono- or poly-substituted by hydroxy, halogen, C₁-C₆alkyl, C₁-C₆alkoxy, C₁-C₆alkylthio, C₁-C₆alkylsulfinyl, C₁-C₆alkylsulfonyl, cyano, carbamoyl, carboxy, C₁-C₄alkoxycarbonyl or phenyl; it being possible for phenyl to be substituted by halogen, C₁-C₃alkyl, C₁-C₃haloalkyl, hydroxy, C₁-C₃alkoxy, C₁-C₃haloalkoxy, cyano or nitro; or
R₉ is phenyl or heteroaryl, each of which may be mono-, di- or tri-substituted by halogen, C₁-C₃alkyl, C₁-C₃haloalkyl, hydroxy, C₁-C₃alkoxy, C₁-C₃haloalkoxy, cyano or nitro;
R₁₀ is halogen, cyano, rhodano, hydroxy, C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, C₂-C₆alkenylthio, C₂-C₆alkynylthio, C₁-C₆alkylsulfonyloxy, phenylsulfonyloxy, C₁-C₆alkylcarbonyloxy, benzoyloxy, C₁-C₄alkoxy-carbonyloxy, C₁-C₆alkylcarbonyl, C₁-C₄alkoxycarbonyl, benzoyl, aminocarbonyl, C₁-C₄alkyl-aminocarbonyl, C₃-C₆cycloalkyl, phenyl, phenoxy, phenylthio, phenylsulfinyl or phenyl-sulfonyl; it being possible for the phenyl-containing groups in turn to be mono- or polysubstituted by halogen, C₁-C₃alkyl, C₁-C₃haloalkyl, hydroxy, C₁-C₃alkoxy, C₁-C₃haloalkoxy, cyano or nitro;
R₁₁ and R₁₇ are each independently of the other hydrogen, C₁-C₄alkyl, C₂-C₄alkenyl, C₂-C₄alkynyl, C₁-C₄alkylthio, C₁-C₄alkylsulfinyl, C₁-C₄alkylsulfonyl, C₁-C₄alkoxycarbonyl, hydroxy, C₁-C₄alkoxy, C₃-C₄alkenyloxy, C₃-C₄alkynyloxy, hydroxy-C₁-C₄alkyl, C₁-C₄alkyl-sulfonyloxy-C₁-C₄alkyl, halogen, cyano or nitro;
or, when A₂ is C(R₁₄R₁₅)m, R₁₇ together with R₁₁ forms a direct bond, or a C₁-C₃alkylene or an ethenylene bridge;
R₁₂ and R₁₈ are each independently of the other hydrogen, C₁-C₄alkyl or C₁-C₄alkylthio, C₁-C₄alkylsulfinyl or C₁-C₄alkylsulfonyl;
or R₁₂ together with R₁₁, and/or R₁₈ together with R₁₇ form a C₂-C₅alkylene chain which can be interrupted by —O—, —C(O)— or —S(O)ₜ—;
t is 0, 1 or 2;
R₁₃ and R₁₉ are each independently of the other hydrogen, C₁-C₄alkyl, C₁-C₄haloalkyl, C₃-C₄alkenyl, C₃-C₄alkynyl or C₁-C₄alkoxy;
R₁₄ is hydrogen, hydroxy, C₁-C₄alkyl, C₁-C₄haloalkyl, C₁-C₃hydroxyalkyl, C₁-C₄alkoxy-C₁-C₃-alkyl, C₁-C₄alkylthio-C₁-C₃alkyl, C₁-C₄alkylcarbonyloxy-C₁-C₃alkyl, C₁-C₄alkylsulfonyloxy-C₁-C₃alkyl, tosyloxy-C₁-C₃alkyl, di(C₁-C₄alkoxy)-C₁-C₃alkyl, C₁-C₄alkoxycarbonyl, C₃-C₅-oxacycloalkyl, C₃-C₅thiacycloalkyl, C₃-C₄dioxacycloalkyl, C₃-C₄dithiacycloalkyl, C₃-C₄oxa-thiacycloalkyl, formyl, C₁-C₄alkoxyiminomethyl, carbamoyl, C₁-C₄alkylaminocarbonyl or di-(C₁-C₄alkyl)aminocarbonyl;
or R₁₄ together with R₁₁, R₁₂, R₁₃, R₁₇, R₁₈, R₁₉ or, when m is 2, also together with R₁₅, forms a direct bond or a C₁-C₄alkylene bridge;
R₁₅ is hydrogen, C₁-C₃alkyl or C₁-C₃haloalkyl;
R₁₆ is hydrogen, C₁-C₃alkyl, C₁-C₃haloalkyl, C₁-C₄alkoxycarbonyl, C₁-C₄alkylcarbonyl or N,N-di(C₁-C₄alkyl)aminocarbonyl; or
Q is a group Q₂ 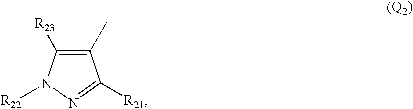
wherein
R₂, and R₂₂ are hydrogen or C₁-C₄alkyl;
R₂₃ is hydroxy, O⁻M⁺, wherein M⁺ is an alkali metal cation or ammonium cation; or is halogen, C₁-C₁₂alkylsulfonyloxy, C₁-C₁₂alkylthio, C₁-C₁₂alkylsulfinyl, C₁-C₁₂alkylsulfonyl, C₁-C₁₂halo-alkylthio, C₁-C₁₂haloalkylsulfinyl, C₁-C₁₂haloalkylsulfonyl, C₁-C₆alkoxy-C₁-C₆alkylthio, C₁-C₆-alkoxy-C₁-C₆alkylsulfinyl, C₁-C₆alkoxy-C₁-C₆alkylsulfonyl, C₃-C₁₂alkenylthio, C₃-C₁₂alkenyl-sulfinyl, C₃-C₁₂alkenylsulfonyl, C₃-C₁₂alkynylthio, C₃-C₁₂alkynylsulfinyl, C₃-C₁₂alkynylsulfonyl, C₁-C₄alkoxycarbonyl-C₁-C₄alkylthio, C₁-C₄alkoxycarbonyl-C₁-C₄alkylsulfinyl, C₁-C₄alkoxy-carbonyl-C₁-C₄alkylsulfonyl, benzyloxy or phenylcarbonylmethoxy; it being possible for the phenyl-containing groups to be mono- or polysubstituted by halogen, C₁-C₃alkyl, C₁-C₃haloalkyl, hydroxy, C₁-C₃alkoxy, C₁-C₃haloalkoxy, cyano or nitro; or
Q is a group Q₃ 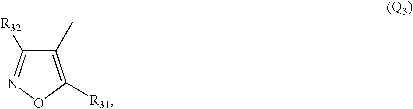
wherein
R₃₁ is C₁-C₆alkyl, C₁-C₆haloalkyl, C₃-C₆cycloalkyl or halo-substituted C₃-C₆cycloalkyl;
R₃₂ is hydrogen, C₁-C₄alkoxycarbonyl, carboxy or a group S(O)ₛR₃₃;
R₃₃ is C₁-C₆alkyl or C₁-C₃alkylene, which can be substituted by halogen, C₁-C₃alkoxy, C₂-C₃alkenyl or by C₂-C₃alkynyl; and
s is 0, 1 or 2; or
Q is a group Q₄ 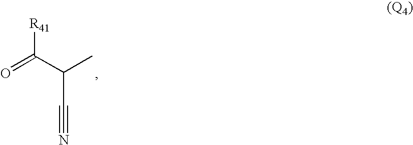
wherein
R₄₁ is C₁-C₆alkyl, C₁-C₆haloalkyl, C₃-C₆cycloalkyl or halo-substituted C₃-C₆cycloalkyl;
and the agrochemically acceptable salts and all stereoisomers and tautomers of compounds of formula I.

2. A compound of formula I according to claim 1, wherein
X₁ is nitrogen or CR₁;
X₂ is nitrogen, if X₁ is CR₁; or is CR₂, if X1 is nitrogen;
and Q, R₁, R₂, R₄ and R₅ are as defined under formula I in claim 1.

3. A compound of formula I according to claim 1, wherein
X₁ is NR₅₁, if X₂ is C(O); or is C(O), if X₂ is NR₅₂;
X₂ is NR₅₂, if X₁ is C(O); or is C(O), if X₁ is NR₅₁;
and Q, R₄, R₅, R₅₁ and R₅₂ are as defined under formula I in claim 1.

4. A compound of formula I according to claim 1 represented by the formulae I-1 to I-8 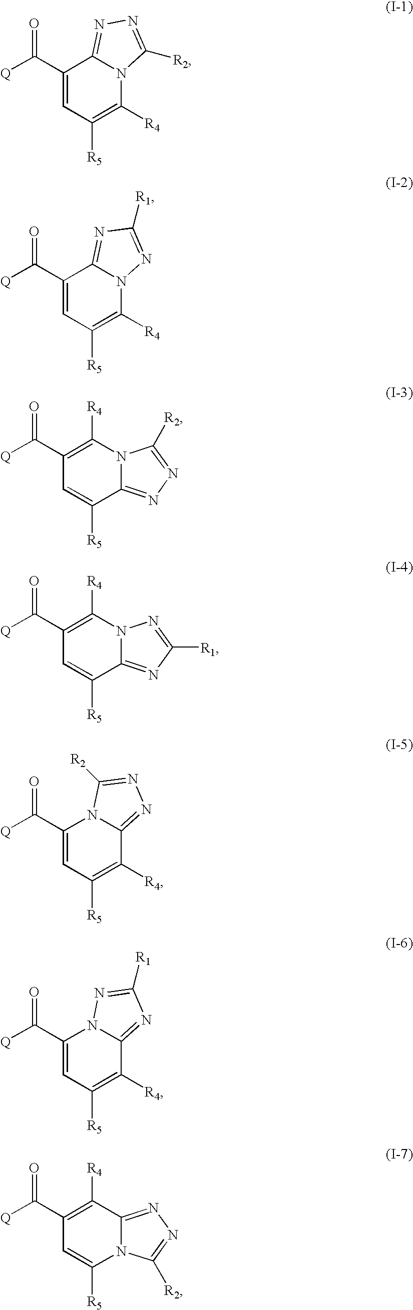 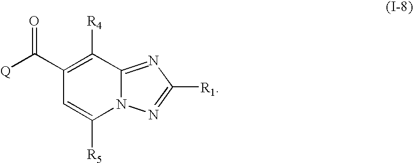
wherein R₁, R₂, R₃, R₄, R₅ and Q have the meanings as given under formula I in claim 1.

5. A compound of formula I-1 according to claim 4, wherein
Q is Q₁ or Q₂;
R₂ is hydrogen, halogen, or a group —X₆, —X₅—X₆ or —X₄—X₅—X₆;
X₄ is C₁-C₆alkylene, C₂-C₆alkenylene or C₂-C₆alkynylene chain, which can be mono-, di- or tri-substituted by halogen, hydroxy, C₁-C₆alkoxy, C₃-C₆cycloalkyloxy, C₁-C₆alkoxy-C₁-C₆alkoxy, C₁-C₆alkoxy-C₁-C₆alkoxy-C₁-C₆alkoxy or C₁-C₂alkylsulfonyloxy; or by a bivalent C₁-C₈alkylene group which may be interrupted by 1 to 2 oxygen atoms, sulphur or NRa₂₆, said bivalent C₁-C₈ alkylene group can be substituted by halogen, hydroxy, amino, formyl, carboxy, nitro, cyano, mercapto, carbamoyl, C₁-C₆alkoxy, C₁-C₆alkoxycarbonyl, C₁-C₆-alkylaminocarbonyl, C₁-C₆-dialkylaminocarbonyl, C₂-C₆alkenyl, C₂-C₆haloalkenyl, C₂-C₆alkynyl, C₂-C₆haloalkynyl, C₂-C₆alkenyloxy, C₂-C₆alkynyloxy, C₁-C₆haloalkoxy, C₂-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆alkoxy, C₁-C₆alkylcarbonyl, C₁-C₆alkylthio, C₁-C₆alkylsulfinyl, C₁-C₆alkylsulfonyl, C₁-C₆haloalkylthio, C₁-C₆haloalkylsulfinyl or C₁-C₆halo-alkylsulfonyl, C₁-C₆alkylthiocarbonyl, C₁-C₆alkylamino, di(C₁-C₆alkyl)amino, C₁-C₄alkylsulfonyloxy, C₁-C₄alkylcarbonylamino, N(C₁-C₄alkyl)-C₁-C₄alkylcarbonylamino, C₁-C₄alkoxycarbonylamino, N(C₁-C₄alkyl)-C₁-C₄alkoxycarbonylamino, C₁-C₄alkylsulfonylamino, N(C₁-C₄alkyl)-C₁-C₄alkylsulfonylamino, OSO₂-C₁-C₄-alkyl, rhodano, tri(C₁-C₄alkyl)silyl or di(C₁-C₄alkoxy)phosphono;
X₅ is oxygen, —OC(O)—, —OC(O)O—, —OC(O)N(R₃)—, OS(O)₂—, thio, sulfonyl, —C(O)O—, —C(O)—, —C(O)N(R₃)—, —N(R₃)C(O)—, —N(R₃)C(O)N(R₃)— or —N(R₃)SO₂N(R₃)—;
X₆ is C₁-C₆alkyl which may be mono-, di- or tri-substituted by halogen, hydroxy, amino, formyl, carboxy, nitro, cyano, mercapto, carbamoyl, C₁-C₆alkoxy, C₁-C₆alkoxycarbonyl, C₁-C₆-alkylaminocarbonyl, C₁-C₆-dialkylaminocarbonyl, C₂-C₆alkenyl, C₂-C₆haloalkenyl, C₂-C₆alkynyl, C₂-C₆haloalkynyl, C₂-C₆alkenyloxy, C₂-C₆alkynyloxy, C₁-C₆haloalkoxy, C₂-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆alkoxy, C₁-C₆alkylcarbonyl, C₁-C₆alkylthio, C₁-C₆alkylsulfinyl, C₁-C₆alkylsulfonyl, C₁-C₆haloalkylthio, C₁-C₆haloalkylsulfinyl or C₁-C₆halo-alkylsulfonyl, C₁-C₆alkylthiocarbonyl, C₁-C₆alkylamino, di(C₁-C₆alkyl)amino, C₁-C₄alkylsulfonyloxy, C₁-C₄alkylcarbonylamino, N(C₁-C₄alkyl)-C₁-C₄alkylcarbonylamino, C₁-C₄alkoxycarbonylamino, N(C₁-C₄alkyl)-C₁-C₄alkoxycarbonylamino, C₁-C₄alkylsulfonylamino, N(C₁-C₄alkyl)-C₁-C₄alkylsulfonylamino, OSO₂-C₁-C₄-alkyl, rhodano, tri(C₁-C₄alkyl)silyl or di(C₁-C₄alkoxy)phosphono;
or X₆ is a three- to ten-membered mono- or bicyclic ring system, which may be aromatic or saturated or partially saturated and may contain from 1 to 4 hetero atoms selected from aromatic nitrogen, oxygen, sulfur, —S(O)—, —S(O)₂—, —N(Ra₂₆)—, —C(O)— and/or C(═NORa₇), and each ring system may contain not more than two oxygen atoms and not more than two sulfur atoms, and the ring system can itself be mono-, di- or tri-substituted by C₁-C₆alkyl, C₁-C₆haloalkyl, C₂-C₆alkenyl, C₂-C₆haloalkenyl, C₂-C₆alkynyl, C₂-C₆haloalkynyl, hydroxy, C₁-C₆alkoxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di(C₁-C₂alkyl)aminosulfonyl, di(C₁-C₄alkyl)amino, halogen, cyano, nitro, phenyl, benzyloxy and/or by benzylthio, it being possible for phenyl groups in turn to be substituted on the phenyl ring by C₁-C₃alkyl, C₁-C₃haloalkyl, C₁-C₃alkoxy, C₁-C₃haloalkoxy, C₁-C₃alkylsulfonyl C₁-C₃haloalkylsulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl , halogen, cyano or nitro;
R₄ is halogen, C₁-C₆-alkyl, C₁-C₆-haloalkyl, C₂-C₆-alkinyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₁-C₆-haloalkylsulfinyl, triazolyl, furyl or phenyl, it being possible for phenyl in turn to be substituted by C₁-C₃alkyl, C₁-C₃haloalkyl, C₁-C₃alkoxy, C₁-C₃haloalkoxy, C₁-C₃alkylsulfonyl C₁-C₃haloalkylsulfonyl, aminosulfonyl, C₁-C₂alkylaminosulfonyl, di(C₁-C₂alkyl)aminosulfonyl, di(C₁-C₄alkyl)amino, C₁-C₄alkoxycarbonyl, halogen, cyano or nitro; and
R₅ is is hydrogen, halogen, C₁-C₃alkyl, C₁-C₃haloalkyl or C₁-C₃alkoxy.

6. A compound of formula I according to claim 1 represented by the compounds selected from formulae I-1a, I-2a, I-1b, I-1c, I-1d, I-1e, I-2b, I-2d, I-5 (wherein Q is 5-Me-2,6-cyclohexanedione) and I-1j (wherein R₂₄ is C₁-C₆alkyl, and R₂₅ is hydrogen, or R₂₄ and R₂₅ together are C₂-C₆alkylen), 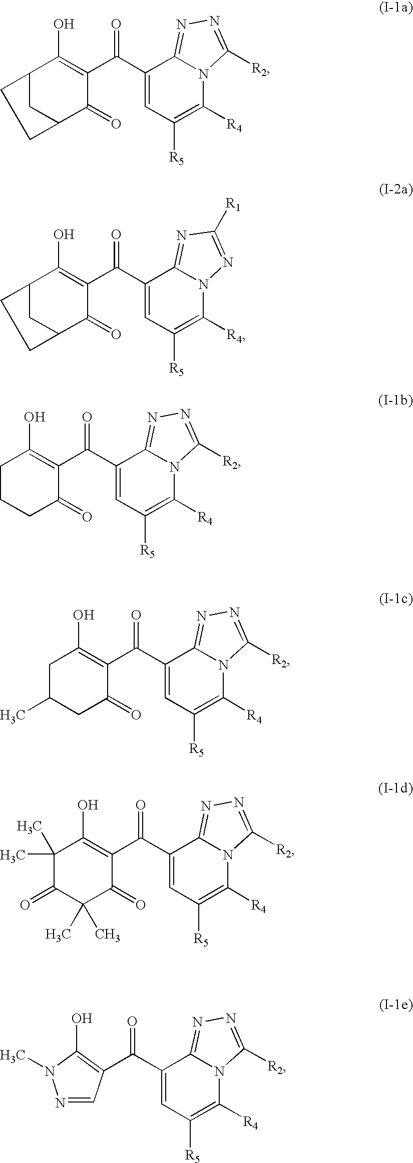 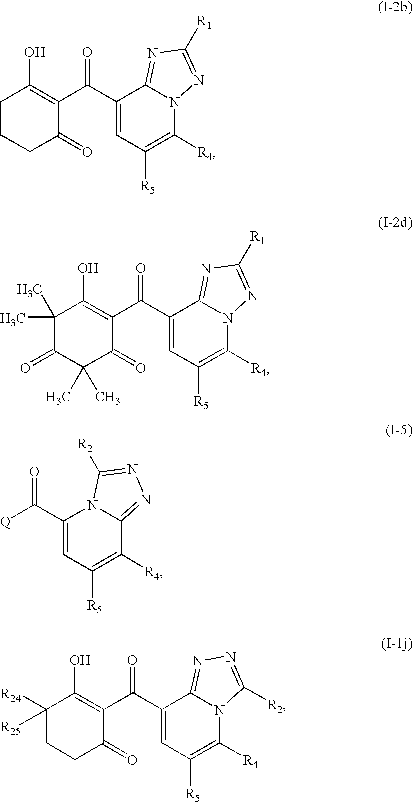
wherein
R₁ and R₂, independently from each other, are hydrogen, halogen, C₁-C₆alkyl, C₁-C₆haloalkyl, hydroxy-C₁-C₆alkyl, C₁-C₆alkoxy-C₁-C₆alkyl, C₁-C₆alkoxycarbonyl-C₁-C₆alkyl, phenoxycarbonyl-C₁-C₆alkyl, C₁-C₆alkylthio-C₁-C₆alkyl, C₁-C₆alkylsulfonyl-C₁-C₆alkyl, phenylsulfonyl-C₁-C₆alkyl, C₁-C₆sulfinyl-C₁-c₆alkyl, C₃-C₆cycloalkyl, C₂-C₆alkenyl, C₂-C₆alkinyl, (2-tetrahydrofuryl)-C₁-C₆alkoxy-C₁-C₆alkyl, amino, di-(C₁-C₆alkyl)amino, C₁-C₆alkylcarbonylamino, C₁-C₆alkoxy, C₂-C₆alkinyloxy, C₁-C₆alkoxy-C₁-C₆alkoxy, C₁-C₆haloalkoxy, phenyl, phenoxy, 4-chlorophenyl, mercapto, phenyl-C₁-C₆alkylthio, C₁-C₆alkylsulfonyl, di-(C₁-C₆alkyl)aminosulfonyl, phenylsulfonyl, Phenyl-C₁-C₆alkylsulfinyl, C₁-C₆alkylsulfinyl, phenylsulfinyl, phenylthio, 2-furyl, 2-pyridyl, 3-pyridyl or 4-pyridyl;
R₄ is C₁-C₆haloalkyl, C₁-C₆alkyl, cyano or triazolyl; and
R₅ is hydrogen, C₁-C₆alkyl, halogen or C₁-C₆alkoxy.

7. A compound according to claim 6, wherein
R₁ and R₂, independently from each other, are hydrogen, C₁-C₆alkyl, C₁-C₆cycloalkyl, C₁-C₆alkoxyalkyl or 4-chlorophenyl;
R₄ is C₁-C₆haloalkyl; and
R₅ is hydrogen.

8. A compound of formula II 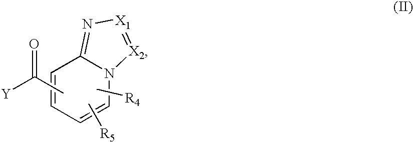
wherein Y is fluorine, chlorine, cyano, hydroxy, C₁-C₄alkoxy, allyloxy, benzyloxy, phenoxy, or benzyloxy, phenoxy substituted by C₁-C₄-alkyl, halogen, cyano, nitro, C₁-C₄-alkoxycarbonyl, C₁-C₃-alkylsulfinyl or C₁-C₃-alkylsulfonyl, or Y is a group 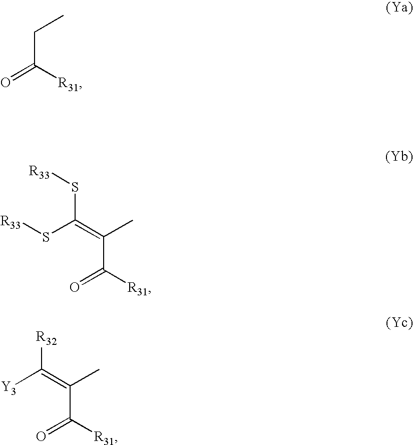
or a group Q₀, wherein Q₀ is accordingly a group Q linked to oxygen, Y₃ is a leaving group and Q, X₁, X₂, R₄, R₅, R₃₁, R₃₂ and R₃₃ are as defined above for formula I in claim 1.

9. A compound of formula IId 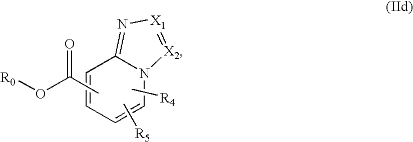
wherein R₀ is hydroxy and X₁, X₂, R₄ and R₅ are as defined for formula I in claim 1 with the proviso that R₄ is different from hydrogen if R₅ is hydrogen or chlorine.

10. A herbicidal composition which comprises a herbicidally effective amount of a compound of formula I.

11. A method of controlling grasses and weeds in crops of useful plants, which comprises applying a herbicidally effective amount of a compound of formula I, or of a composition comprising such a compound, to the plants or to the locus thereof.
